SEQUENCE LISTING (46 sequences)

SEQ ID NO: 1 - protein, 844 aa, HIV-1
  VARIANT (0)...(0): Xaa = any amino acid
MKVRGIQRNYQHLLTWGTMILGILGFCNAAENLWVTVYYGVPVWKDAETTLFCASDAKAYSTEKHXVWATHACVPTDPNPQEIHLENVTEEFNMWKNNMVDQMHEDIISLWDQSLKPCVKLTPLCVTLNCNNINDSKIIDKEMKGQIKNCSYNMTTELRDKKKQVYSLFYKVDVVPIEENNGNSNSSEYRLINCNTSAITQACPKVSFEPIPIHYCAPAGFAILKCRDREFNGTGPCKNVSTVQCTHGIKPVVSTQLLLNGSLSEKEIIIRAENITNNAKIIIVQLNESVWINCSRPNNNTRKSVRIGPGQAFFATGEIIGDIRQAQCNISRSKWNETLQRVKGKLKDYFKNNITFDNSSGGDLEITTHSFNCRGEFFYCNTTGLFNESLLNNSTNENITLPCKIKQIVRMWQRVGQAMYAPPIAGKLECRSNITGLLLTRDGGKENQTENNPEIFRPGGGDMRDNWRSELYKYKVVKIEPLGVAPTKARRRVVEREKRAVGMGALFLGFLGTAGSTMGAASITLTVQARXLLSGIVQQQNNLLKAIEAQQHLLKLTVWGIKQLQARVLAVERYLKDQQLLGIWGCSGKLICTTNVPWNSSWSNKTEGEIWDNMTWLQWDKEISNYTQIIYNLLEESQNQQEKNEQDLLALDKWDSLWNWFSISKWLWYIKIFIMIVGGLIGLRIVFAVISVINRVRQGYSPLSFQIHTPNPRGPDRPGRIEEEGGEPGRDRSTRLVNGFLALVWDDLRSLFLFSYHHLRDFILIAARTVELLGRRGWEALKYLGNLLLYWGRELKISAINLLDTIAIAVAGWTDRAIEVGQRIXXAVLNIPRRIRQXFERALL

SEQ ID NO: 2 - protein, 861 aa, HIV-1
  VARIANT (0)...(0): Xaa = any amino acid
MRVMGXQMNWQGLWRWGTMILGMIIICSAADNLWVTVYYGVPVWRDADTTLFCASDAKAYDXEAHNVWATHACVPTDPNPQEXHLKNVTEEFNMWKNNMVEQMHTDIISLWDQXLKPCVKLTPXCVTLBCVNNITFYNNSSPQFTNSSDMRNXSFNMTTELRDKXQXVHSLFYKLDIVPIGGTNNXDGQYRLINCNTSAITQACPKVSFEPIPIHYXTPAGFAILLCNDKXFNGTGPCKNVSSVQCTHGIRPVVSTQLLLNGSLAEEEVRIRSEXLTDNAKXIIVQLXXPVQINCTRPNNNTRKSVHIGPGQAFFATGDIIGDIREAFCEVNTKKWNATLQKVAXQLKNYFNKTIIFNSSSGGDLEITTHSFNCGGEFFYCNTSRLXNSTWMFNGTWGNNTVEXEKSNDTLXLPXKIKQIIRMWQRAGQAMYAPPIQGVIXCVSNITGLLLTRBGGKXSNESETFRPEGGNMRDNWRSELYKYKVVKIEPLGVAPTXAKRRVVQREXRAAIGLGAVFXGFLGAAGSTMGAASMTLTVQARQLLSGIVQQQSNLLXAIEAQQHLLKLTVWGIKQLQARVLAVERYLRDQQLLGIWGCSGKLICTTAVPWNSTWSKKNQSEIWDNMTWLQWDKEISNYTDIIYNLLEEXQNQQEKNEQDLLALDKWAXLWNWFDISKWLWYIKIFIMIVGGLIGLRIVFAVLSIINRVRQGYSPLSFQIHXPNPEALDRPEXIEEEGGEQGRDRSIRLVSGFLALAWDDLRSLCLFSYHRLRDFILIVTRTVELLGHSSLKGLRLGWEGLKYLGNLLTXWGQELKXSAINLLDTXAIAVAGWTDRVIEIVQRICRAFLNIPRRIRQGLERILL

SEQ ID NO: 3 - protein, 863 aa, HIV-1
MIVMGTQRNYQHLLRWGTMILGLIIICSAADNLWVTVYYGVPVWKDAETTLFCASDAKAYETEKHNVWATHACVPTDPNPQEIPLENVTEEFNMWKNKMVEQMHTDIISLWDQSLQPCVKLTPLCVTLNCTDATNGTIGNITDEMKGEIKNCSFNITTEIRDKKQKVYSLFYRLDVVPIEPDSSNSSRNSSEYRLINCNTSAITQACPKVSFEPIPIHYCAPAGFAILKCRDKEFNGTGKCKNVSTVQCTHGIKPVVSTQLLLNGSLAEGEVRIRSENITNNAKTIIVQLVEPVRINCTRPNNNTRESVRIGPGQAFFATGDIIGDIRQAHCNVSRSQWNKTLQQVAAQLGEHFKNKAITFNSSSGGDLEITTHSFNCGGEFFYCNTSGLFNSTWKANNGTWKANISESNNTEITLQCRIKQIINMWQRTGQAIYAPPIQGVIRCESNITGLLLTRDGGEGNNESEIFRPGGGDMRDNWRSELYKYKVVKIEPLGVAPTRARRRVVGREKRAVGIGAVFLGFLGAAGSTMGAASITLTVQARQLLSGIVQQQSNLLRAIEAQQHMLKLTVWGIKQLQARVLAVERYLRDQQLLGIWGCSGKLICTTNVPWNSSWSNKSHDEIWNNMTWLQWDKEISNYTNLIYSLIEESQNQQEKNEQDLLALDKWASLWNWFDISKWLWYIKIFIMIVGGLIGLRIVFAVLAVIKRVRQGYSPVSFQIHNPNPGGLDRPGRIEEEGGEPGRGRSIRLVSGFLALAWDDLRNLCLFSYHRLRDFALIVARTVELLGHSSLKGLRLGWEGLKYLWNLLVYWSQELKTSAINLVDTIAIAVAGWTDRVIEIGQGIGRAFLHIPRRIRQGLERALL

SEQ ID NO: 4 - DNA, 2592 bp, HIV-1
atgatagtgatggggacacagaggaattatcagcacttattgagatggggaactatgatcttgggattgataataatctgtagtgctgcagacaacttgtgggttactgtctattatggggtacctgtgtggaaagatgcagagaccaccttattttgtgcatcagatgctaaagcatatgagacagaaaagcataatgtttgggctacacatgcctgtgtgcccacagaccccaacccacaagaaatacctttggaaaatgtgacagaagagtttaacatgtggaaaaataaaatggtagaacaaatgcatacagatataatcagtctatgggaccaaagcctacagccatgtgtaaagttaacccctctctgtgttactttaaattgtacggatgctactaatggtacgattggcaacatcaccgatgaaatgaagggagaaataaaaaactgctctttcaatataaccacagaaataagggataagaaacagaaagtatattcacttttttatagacttgatgtagtaccaatagagccagatagtagtaatagtagtagaaacagtagtgagtatagattaataaattgtaatacctcagccattacacaagcctgcccaaaggtaagctttgagccaattcccatacattattgtgccccagctggttttgcgatcctgaagtgtagggataaagagttcaatggaacagggaaatgcaagaatgtcagcacagtccaatgcacacatggaatcaagccagtagtatcaactcaactgctgttaaatggcagtctagcagaaggagaggtaagaattagatctgaaaatatcacaaacaatgccaaaactataatagtacaacttgtcgagcctgtgagaattaattgtactagacctaataacaatacaagagagagtgtgcgtatagggccaggacaagcattctttgcaacaggtgacataataggggatataagacaagcacattgtaatgtcagtagatcacaatggaataagactttacaacaggtagctgcacaattaggagaacactttaaaaacaaagcaataacatttaacagttcctcaggaggagatctagaaatcacaacacatagttttaattgtggaggagaatttttctattgtaatacatcaggtctgttcaatagcacctggaaggccaacaatggcacctggaaggccaacatatcagagtcaaataacacggagataactctccaatgcagaataaagcaaattataaatatgtggcagagaacaggacaagcaatatatgcccctcccatccagggagtgataaggtgtgaatcaaacatcacaggactactgttaacaagagatggtggggaggggaacaatgaaagtgagatcttcagacctggaggaggagatatgagggacaactggagaagtgaattatataagtataaagtagtaaaaattgaaccactaggagtagcacccaccagggcaaggagaagagtggtgggaagagaaaaaagagcagttggaataggagctgttttccttgggttcttaggagcagcaggaagcactatgggcgcggcgtcaataacgctgacggtacaggccaggcaattattgtctggcatagtgcaacagcaaagcaatttgctgagggctatagaggctcaacaacatatgttgaaactcacggtctggggcattaaacagctccaggcaagagtccttgctgtggagagatacctaagggatcaacagctcctaggaatttggggctgctctggaaaactcatctgcaccactaatgtgccctggaactctagttggagtaataaatctcatgatgaaatatggaacaacatgacctggctgcaatgggataaagaaattagcaattacacaaacctaatatatagtctaattgaagaatcgcaaaaccagcaggaaaagaatgaacaagatttattggcattggacaagtgggcaagtctgtggaattggtttgacatatcaaagtggctgtggtatataaaaatatttataatgatagtaggaggtttaataggattaagaatagtttttgctgtgcttgctgtaataaagagagttaggcagggatactcacctgtgtcatttcagatccacaacccaaacccagggggtctcgacaggcccggaagaatcgaagaagaaggtggagagccaggcagaggcagatcgattcgattagtgagcggattcttagcacttgcctgggacgatctgaggaacctgtgcctcttcagctaccatcgcttgagagacttcgccttgattgttgcgaggactgtggaacttctgggacacagcagtctcaaggggttgagactggggtgggaaggcctcaagtatctgtggaatctcctggtatactggagtcaggaactgaaaactagtgctattaatttggttgatactatagcaatagcagtagctggctggacagatagggttatagaaataggacaaggaattggtagagcttttctccacatacctagaagaatcagacagggcttagaaagggcattgctgtaa

SEQ ID NO: 5 - protein, 847 aa, HIV-1
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIKIFIMIVGGLVGLRLVFTVLSIVNRVRQGYSPLSFQTLLPAPRGPDRPEGIEEEGGERDRDRSGRLVNGFLALIWVDLRSLCLFSYHRLRDLLLTVTRIVELLGRRGWEVLKYWWNLLQYWSQELKNSAVSLLNATAIAVAEGTDRIIEALQRTYRAILHIPTRIRQGLERALL

SEQ ID NO: 6 - DNA, 2541 bp, HIV-1
atgagagtgaaggggatcaggaagagttatcagtacttgtggaaagggggcaccttgctccttgggatattaatgatctgtagtgctgtagaaaagttgtgggtcacagtctattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtaacagaacattttaacatgtggaaaaataacatggtagaacagatgcaggaggatataatcagtttatgggatcaaagcctaaagccatgtgtaaaattaaccccactctgtgttactttaaattgcaaggatgtgaatgctactaataccactaatgatagcgagggaacgatggagagaggagaaataaaaaactgctctttcaatatcaccacaagcataagagatgaggtgcagaaagaatatgctcttttttataaacttgatgtagtaccaatagataataataataccagctataggttgataagttgtgacacctcagtcattacacaggcctgtccaaagatatcctttgagccaattcccatacattattgtgccccggctggttttgcgattctaaagtgtaatgataagacgttcaatggaaaaggaccatgtaaaaatgtcagcacagtacaatgtacacatggaattaggccagtagtatcaactcaactgctgctaaatggcagtctagcagaagaagaggtagtaattagatctgacaatttcacgaacaatgctaaaaccataatagtacagctgaaagaatctgtagaaattaattgtacaagacccaacaacaatacaagaaaaagtatacatataggaccagggagagcattttatactacaggagaaataataggagatataagacaagcacattgtaacattagtagagcaaaatggaatgacactttaaaacagatagttataaaattaagagaacaatttgagaataaaacaatagtctttaatcactcctcaggaggggacccagaaattgtaatgcacagttttaattgtggaggagaatttttctactgtaattcaacacaactgtttaatagtacttggaataataatactgaagggtcaaataacactgaaggaaatactatcacactcccatgcagaataaaacaaattataaacatgtggcaggaagtaggaaaagcaatgtatgcccctcccatcagaggacaaattagatgttcatcaaatattacagggctgctattaacaagagatggtggtattaatgagaatgggaccgagatcttcagacctggaggaggagatatgagggacaattggagaagtgaattatataaatataaagtagtaaaaattgaaccattaggagtagcacccaccaaggcaaagagaagagtggtgcaaagagaaaaaagagcagtgggaataggagctgtgttccttgggttcttgggagcagcaggaagcactatgggcgcagcgtcaatgacactgacggtacaggccagactattattgtctggtatagtgcaacagcagaacaatttgctgagggctattgaggcgcaacagcgtatgttgcaactcacagtctggggcatcaagcagctccaggcaagagtcctggctgtggaaagatacctaggggatcaacagctcctggggatttggggttgctctggaaaactcatttgcaccactgctgtgccttggaatgctagttggagtaataaatctctggataggatttggaataacatgacctggatggagtgggaaagagaaattgacaattacacaagcgaaatatacaccctaattgaagaatcgcagaaccaacaagaaaagaatgaacaagaattattggaattagataaatgggcaagtttgtggaattggtttgacataacaaaatggctgtggtatataaaaatattcataatgatagtaggaggcttagtaggtttaagactagtttttactgtactttctatagtgaatagagttaggcagggatactcaccattatcgtttcagaccctcctcccagccccgaggggacccgacaggcccgaaggaatcgaagaagaaggtggagagagagacagagacagatccggacgattagtgaacggattcttagcacttatctgggtcgacctgcggagcctgtgcctcttcagctaccaccgcttgagagacttactcttgactgtaacgaggattgtggaacttctgggacgcagggggtgggaagtcctgaaatattggtggaatctcctacagtattggagtcaggaactaaagaatagtgctgttagcttgctcaatgccacagccatagcagtagctgaggggacagataggattatagaagcattacaaagaacttatagagctattctccacatacctacaagaataagacagggcttggaaagggctttgcta

SEQ ID NO: 7 - protein, 855 aa, HIV-1
MRVKEKYQHLWRWGWRWGTMLLGMLMICSATEKLWVTVYYGVPVWKEATTTLFCASDAKAYDTEVHNVWATHACVPTDPNPQEVELENVTENFNMWKNNMVEQMHEDIISLWDQSLKPCVKLTPLCVTLNCTDLRNATNGNDTNTTSSSREMMGGGEMKNCSFKITTNIRGKVQKEYALFYELDIVPIDNNSNNRYRLISCNTSVITQACPKISFEPIPIHYCAPAGFAILKCKDKKFNGKGPCSNVSTVQCTHGIRPVVSTQLLLNGSLAEEEVVIRSENFADNAKTIIVQLNESVEINCTRPNNNTRKSIHIGPGRALYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQKVGRAMYAPPIRGQIRCSSNITGLLLTRDGGPEDNKTEVFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQHLLQLTVWGIKQLQARVLAVERYLRDQQLLGIWGCSGKLICTTAVPWNASWSNKSLNKIWDNMTWMEWDREINNYTSIIYSLIEESQNQQEKNEQELLELDKWASLWNWFEITEWLWYIKIFIMIIGGLIGLRIVFSVLSIMNRVRQGYSPLSFQTHLPASRGPDRPGGIEEEGGERDRDRSGRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 8 - DNA, 2568 bp, HIV-1
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaaccaccactctattttgtgcatcagatgctaaagcatatgatacagaggtacataatgtttgggccacacatgcctgtgtacccacagaccccaacccacaagaagtagaattggaaaatgtgacagaaaattttaacatgtggaaaaataacatggtagaacagatgcatgaggatataatcagtttatgggatcaaagcctaaagccatgtgtaaaattaactccactctgtgttactttaaattgcactgatttgaggaatgctactaatgggaatgacactaataccactagtagtagcagggaaatgatggggggaggagaaatgaaaaattgctctttcaaaatcaccacaaacataagaggtaaggtgcagaaagaatatgcacttttttatgaacttgatatagtaccaatagataataatagtaataatagatataggttgataagttgtaacacctcagtcattacacaggcctgtccaaagatatcctttgagccaattcccatacattattgtgccccggctggttttgcgattctaaagtgtaaagataagaagttcaatggaaaaggaccatgttcaaatgtcagcacagtacaatgtacacatgggattaggccagtagtatcaactcaactgctgttaaatggcagtctagcagaagaagaggtagtaattagatccgaaaatttcgcggacaatgctaaaaccataatagtacagctgaatgaatctgtagaaattaattgtacaagacccaacaacaatacaagaaaaagtatacatataggaccaggcagagcattatatacaacaggagaaataataggagatataagacaagcacattgtaaccttagtagagcaaaatggaatgacactttaaataagatagttataaaattaagagaacaatttgggaataaaacaatagtctttaagcattcctcaggaggggacccagaaattgtgacgcacagttttaattgtggaggggaatttttctactgtaattcaacacaactgtttaatagtacttggaatgttactgaagagtcaaataacactgtagaaaataacacaatcacactcccatgcagaataaaacaaattataaacatgtggcagaaagtaggaagagcaatgtatgcccctcccatcagaggacaaattagatgttcatcaaatattacagggctgctattaacaagagatggtggtccagaggacaacaagaccgaggtcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctgtgttccttgggttcttgggagcagcaggaagcactatgggcgcagcgtcaatgacgctgacggtacaggccagactattattgtctggtatagtgcaacagcagaacaatctgctgagggctattgaggcgcaacagcatctgttgcaactcacagtctggggcatcaagcagctccaggcaagagtcctggctgtggaaagatacctaagggatcaacagctcctgggaatttggggttgctctggaaaactcatttgcaccactgctgtgccttggaatgctagttggagtaataaatctctgaataagatttgggataacatgacctggatggagtgggacagagaaattaacaattacacaagcataatatatagcttaattgaagaatcgcagaaccaacaagaaaagaatgaacaagaattattagaattagacaaatgggcaagtttgtggaattggtttgaaataacagaatggctgtggtatataaaaatattcataatgataataggaggcttgataggtttaagaatagttttttctgtactttctataatgaatagagttaggcagggatactcaccattatcgtttcagacccacctcccagcctcgaggggacccgacaggcccggaggaatcgaagaagaaggtggagagagagacagagacagatccggtcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 9 - protein, 844 aa, HIV-1
MRVRGIPRNWPQWWIWGILGFWMIIICRVVGNLNLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCTNATAYNNSMHGEMKNCSFNTTTEIRDRKQKAYALFYKPDVVPLNRREENNGTGEYILINCNSSTITQACPKVTFDPIPIHYCAPAGYAILKCNNKTFNGTGPCNNVSTVQCTHGIKPVVSTQLLLNGSLAEEEIIIRSENLTNNIKTIIVHLNKSVEIVCTRPNNNTRKSIRIGPGQTFYATGEIIGNIREAHCNISKSSWTSTLEQVKKKLKEHYNKTIEFKPPSGGDLEVTTHSFNCRGEFFYCNTTKLFSNNSDSNNETITLPCKIKQIINMWQKVGRAMYAPPIEGNITCKSNITGLLLTRDGGKNTTNEIFRPGGGNMKDNWRSELYKYKVVEIEPLGVAPTKSKRRVVEREKRAVGLGAVLLGFLGAAGSTMGAASITLTVQARQLLSGIVQQQSNLLRAIEAQQHMLQLTVWGIKQLQTRVLAIERYLKDQQLLGLWGCSGKIICTTAVPWNSSWSNKSQEDIWDNMTWMQWDREISNYTGTIYRLLEDSQNQQEKNEKDLLALDSWKNLWNWFDITNWLWYIKIFIMIVGGLIGLRIIFGVLAIVKRVRQGYSPLSFQTLTPSPRGPDRLGRIEEEGGEQDKNRSIRLVSGFLALAWDDLRSLCLFGYHQLRDFILIAARAAELLGRSSLRGLQRGWEALKYLGNLVQYGGLELKRSAIKLFDTIAIAVAEGTDRILEVIRRICRAIRHIPIRIRQGFEAALL

SEQ ID NO: 10 - DNA, 2535 bp, HIV-1
atgagagtgagggggataccgaggaattggccacaatggtggatatggggcatcttaggcttttggatgataataatttgtagggtggtggggaacttgaacttgtgggtcacagtctattatggggtacctgtgtggaaagaagcaaaaactactctattctgtgcatcagatgctaaagcatatgataaagaagtacataatgtctgggctacacatgcctgtgtacccacagaccccaacccacaagaaatagttttggaaaatgtaacagaaaattttaacatgtggaaaaatgacatggtggatcagatgcatgaggatataatcagtttatgggatcaaagcctaaaaccatgtgtaaagttgaccccactctgtgtcactttaaattgtacaaatgcaactgcctacaataatagcatgcatggagaaatgaaaaattgctctttcaatacaaccacagaaataagagataggaaacagaaagcgtatgcacttttttataaacctgatgtagtgccacttaataggagagaagagaataatgggacaggagagtatatattaataaattgcaattcctcaaccataacacaagcctgtccaaaggtcacttttgacccaattcctatacattattgtgctccagctggttatgcgattctaaagtgtaataataagacattcaatgggacaggaccatgcaataatgtcagcacagtacaatgtacacatggaattaagccagtggtatcaactcaattactgttaaatggtagcctagcagaagaagagataataattagatctgaaaatctgacaaacaatatcaaaacaataatagtccaccttaataaatctgtagaaattgtgtgtacaagacccaacaataatacaagaaaaagtataaggataggaccaggacaaacattctatgcaacaggtgaaataataggaaacataagagaagcacattgtaacattagtaaaagtagctggaccagtactttagaacaggtaaagaaaaaattaaaagaacactacaataagacaatagaatttaaaccaccctcaggaggggatctagaagttacaacacatagctttaattgtagaggagaatttttctattgcaatacaacaaaactgttttcaaacaacagtgattcaaacaacgaaaccatcacactcccatgcaagataaaacaaattataaacatgtggcagaaggtaggacgagcaatgtatgcccctcccattgaaggaaacataacatgtaaatcaaatatcacaggactactattgacacgtgatggaggaaagaatacaacaaatgagatattcagaccgggaggaggaaatatgaaggacaattggagaagtgaattatataaatataaagtggtagaaattgagccattgggagtagcacccactaaatcaaaaaggagagtggtggagagagaaaaaagagcagtgggactaggagctgtactccttgggttcttgggagcagcaggaagcactatgggcgcggcgtcaataacgctgacggtacaggccagacaactgttgtctggtatagtgcaacagcaaagcaatttgctgagagctatagaggcgcaacagcatatgttgcaactcacggtctggggcattaagcagctccagacaagagtcttggctatagagagatacctaaaggatcaacagctcctagggctttggggctgctctggaaaaatcatctgcaccactgctgtgccttggaactccagttggagtaataaatctcaagaagatatttgggataacatgacctggatgcagtgggatagagaaattagtaattacacaggcacaatatataggttacttgaagactcgcaaaaccagcaggagaaaaatgaaaaagatttattagcattggacagttggaaaaacttgtggaattggtttgacataacaaattggctgtggtatataaaaatattcatcatgatagtaggaggcttgataggtttgagaataatttttggtgtactcgctatagtgaaaagagttaggcagggatactcacctttgtcgtttcagacccttaccccaagcccgaggggtcccgacaggctcggaagaatcgaagaagaaggtggagagcaagacaaaaacagatccattcgattagtgagcggattcttagcacttgcctgggacgatctgcggagcctgtgcctcttcggttaccaccaattgagagacttcatattgattgcagcgagagcagcggaacttctgggacgcagcagtctcaggggactgcagagagggtgggaagcccttaagtatctgggaaatcttgtgcagtatgggggtctggagctaaaaagaagtgctattaaactgtttgataccatagcaatagcagtagctgaaggaacagataggattcttgaagtaatccgaagaatttgtagagctatccgccacatacctataagaataagacagggctttgaagcagctttgctataa

SEQ ID NO: 11 - protein, 857 aa, HIV-1
MRVRGTLRNYQQWWIWGVLGFWMLMICNGGGNLWVTVYYGVPVWKEAKTTLLCASDAKAYEREVHNVWATHACVPTDPNPQEIVLGNVTENFNMWKNDMVDQMHEDVISLWDQSLKPCVKLTPLCVTLECRNVSRNVSSYNTYNGSVEEIKNCSFNATPEVRDRKQRMYALFYGLDIVPLNKKNSSENSSEYRLINCNTSAITQACPKVTFDPIPIHYCAPAGYAILKCNNKTFNGTGPCNNVSTVQCTHGIKPVVSTQLLLNGSLAEGEIIIRSENLTNNVKTIIVHLNQSVEIVCTRPNNNTRKSIRIGPGQTFYATGDIIGDIRQAHCNISRDKWNETLQRVGKKLAEHFHNKTIKFASSSGGDLEITTHSFNCRGEFFYCNTSGLFNGTYMPTYMPNGTESNSNSTITIPCRIKQIINMWQEVGRAMYAPPIAGNITCTSNITGLLLVHDGGIKENDTENKTEIFRPGGGDMRDNWRSELYKYKVVEIKPLGVAPTAAKRRVVEREKRAVGIGAVFLGFLGAAGSTMGAASITLTAQARQLLSGIVQQQSNLLRAIEAQQHLLQLTVWGIKQLQTRVLAIERYLKDQQLLGIWGCSGKLICTTAVPWNSSWSNKTQSEIWNNMTWMQWDREVSNYTNIIYSLLEESQNQQEKNEKDLLALDSWKNLWSWFDITNWLWYIKIFIMIVGGLIGLRIIFAVLSIVNRVRQGYSPLSFQTLTPNPRGPDRLGRIEEEGGEQDKDRSIRLVNGFLALAWDDLRNLCLFSYHRLRDFISVAARVVELLGRSSWEALKYLGSLVQYWGLELKKSAISLFDSIAIVVAEGTDRIIELVQGFCRAIRNIPTRIRQGFEAALQ

SEQ ID NO: 12 - DNA, 2574 bp, HIV-1
atgagagtgagggggacactgaggaattatcaacaatggtggatatggggcgtcttaggcttttggatgttaatgatttgtaatgggggaggaaacttgtgggtcacagtctattatggggtacctgtgtggaaagaagcaaaaaccactctactctgtgcatcagatgccaaagcatatgagagggaagtgcataatgtctgggctacacatgcctgtgtacccacagaccccaacccacaagaaatagttttgggaaatgtaacagaaaattttaacatgtggaaaaatgacatggtggatcagatgcatgaggatgtaatcagtttatgggatcaaagcctaaagccatgtgtaaaattgaccccactctgtgtcactttagaatgtagaaatgttagcagaaatgttagcagttataatacctacaatgggagcgtggaggaaataaaaaattgctctttcaatgcaaccccagaagtaagagataggaagcagagaatgtatgctctcttttatggacttgatatagtaccacttaataagaagaactctagtgagaactccagtgagtatagattaataaattgtaatacctcagccataacacaagcctgtccaaaggtcacttttgatccaattcctatacactattgtgctccggctggttatgcgattctaaagtgtaataataagacattcaatgggacaggaccatgcaataatgttagtacagtacaatgtacacatggaattaagccagtagtatcaactcaactactgttaaatggtagcctagcagaaggagagataataattagatctgaaaatctgacaaacaatgtcaaaacaataatagtacatcttaatcaatctgtagaaattgtgtgtacaagacccaataataatacaagaaaaagtataaggataggaccaggacaaacattctatgcaacaggagacataataggagacataagacaagcacattgtaacattagtagagataaatggaatgaaactttacaaagggtaggtaaaaaattagcagaacacttccataataagacaataaaatttgcatcatcctcaggaggggacctagaaattacaacacatagctttaattgtagaggagaatttttctattgtaatacatcaggcctgtttaatggtacatacatgcctacatacatgcctaatggtacagaaagtaattcaaactcaactatcacaatcccatgcagaataaagcaaattataaacatgtggcaggaggtaggacgagcaatgtatgcccctcccattgcaggaaacataacatgtacatcaaatatcacaggactactattggtacatgatggaggaataaaggaaaatgatacagagaataagacagagatatttagacctggaggaggagatatgagggacaattggagaagtgaattatataaatataaagtggtagaaattaagccattgggagtagcacccactgcagcaaaaaggagagtggtggagagagaaaaaagagcagtgggaataggagctgtgttccttgggttcttgggagcagcaggaagcactatgggcgcggcgtcaataacgctgacggcacaggccagacaattgttgtctggtatagtgcaacagcaaagcaatttgctgagggctatagaggcgcaacagcatctgttgcaactcacagtctggggcattaagcagctccagacaagagtcctggctatagagagatacctaaaggatcaacagctcctagggatttggggctgctctggaaaactcatctgcactactgctgtaccttggaactccagttggagtaacaaaactcaaagtgagatttggaataacatgacctggatgcagtgggatagagaagttagtaattacacaaacataatatacagcttgcttgaagaatcgcaaaaccagcaggaaaaaaatgaaaaagatttattagcattggacagttggaaaaatctatggagttggtttgacataacaaattggctgtggtatataaaaatattcataatgatagtaggaggcttgataggtttaagaataatttttgctgtgctctctatagtgaatagagttaggcagggatactcacctttgtcgtttcagacccttaccccgaacccaaggggacccgacaggctcggaagaatcgaagaagaaggtggagagcaagacaaagacagatccattcgattagtgaacggattcttagcacttgcctgggacgatctacggaacctgtgcctcttcagctaccaccgattgagagacttcatatcggtggcagcgagagtggtggaacttctgggacgcagcagttgggaagcccttaaatatctgggaagtcttgtgcagtattggggtctggagctaaaaaagagtgctattagtctgtttgatagcatagcaatagtagtagctgaaggaacagataggattatagaattagtacaaggattttgtagagctatccgcaacatacctacaagaataagacagggctttgaagcagctttgcaataa

SEQ ID NO: 13 - protein, 859 aa, HIV-1
MRVREIERNYLCLWRWGIMLLGMLMTYSVAEKKWVTVYYGVPVWKEATTTLFCASDAKSYKTEVHNIWATHACVPTDPNPREIELENVTENFNMWKNNMVEQMHEDIISLWDQSLKPCVKLTPLCVTLNCTDARRNETRNNITGMENNDQIEMKNCSFNITTKLIDKKKQVHALFYRLDVVQIDNDTSNSNYSNYRLINCNTSAITQACPKVTFEPIPIHYCAPAGFAILKCRDKKFNGTGPCKNVSTVQCTHGIRPVVSTQLLLNGSLAEEEIIIRSENLTNNAKTLIVQLNESVEINCTRPYYNQIRQRTSIGQGQALYTTRVTGDIRKAYCNISKAGWNKTLQQVAKKLGDLFNQTTIIFKPSSGGDPEITTHSFNCGGEFFYCNTSKLFNSAWNDSTWNIGNNNTGSDNETIIIPCRIKQIINMWQGVGKAMYAPPIEGWINCASNITGLLLVRDGGGANDSQNETFRPQGGDMRDNWRSELYKYKVVKIEPLGIAPTKAKRRVVEREKRAIGLGAMFLGFLGAAGSTMGAASLTLTVQARQLLSGIVQHQNNLLMAIEAQQHLLQLTVWGIKQLQARILAVERYLQDQQLLGSWGCSGRHICTTTVPWNSSWSNKSIDDIWNNMTWMEWEKEIDNYTGVIYRLIEESQTQQEKNEQELLQLDKWASLWNWFSITKWLWYIKIFIMIVGGLIGLRIVFTVLSLVNRVRQGYSPLSFQTLFPAPRGPDRPEEIEEGGGEQGRGRSTRLVNGFSTLIWDDLRNLCLFSYHRLRDLILIATRIVELLGRRGWEAIKYLWNLLQYWSQELKTSAISLFNATAVAVAEGTDRVIEVVQRFFRGILNVPTRIRQGLERALL

SEQ ID NO: 14 - DNA, 2580 bp, HIV-1
atgagagtgagggagatagagaggaattatctatgcttgtggagatggggcatcatgctccttgggatgttgatgacatatagtgttgcagagaagaagtgggtcacagtgtattatggggtacctgtgtggaaagaagcaacaaccactctattttgtgcatcagatgctaaatcatataaaacagaggtacataatatctgggctacacatgcctgtgtaccaacagaccccaacccacgagaaatagaactggaaaatgtcacagaaaactttaacatgtggaaaaataacatggtggagcagatgcatgaggatatcatcagtttatgggatcaaagcctaaaaccatgtgtaaaattaaccccactctgtgtcactttaaactgcactgatgcaaggaggaatgagactaggaataatattacaggaatggaaaacaatgatcaaatagaaatgaaaaactgctctttcaatataaccacaaaattaatagataagaagaagcaagtacatgcacttttttatagacttgatgtggtacaaatagataatgatactagtaatagcaactatagcaactatagattaataaattgcaatacctcagccattacacaggcttgtccaaaggtaacttttgagccaattcccatacattattgtgccccagctggttttgcaattctaaagtgtagagataagaagttcaatggaacaggaccatgcaaaaatgtcagcacagtacaatgcacacatggaattaggccagtagtgtcaacccaactgctgttgaatggcagtctagcagaagaagagataataattagatctgaaaatctcacaaacaatgctaaaaccctaatagtacagcttaatgagtctgtagaaatcaattgtacaaggccctactacaaccagataagacaaagaacatctataggacaagggcaagcactctatacaacaagagtaacgggagatataagaaaagcatattgcaatattagtaaagcaggatggaataaaactttacagcaggtagcaaaaaaattaggagacctctttaaccagacaacaataatttttaaaccatcctcgggaggagacccagaaattacaacacacagctttaattgtggaggggaatttttctactgcaatacatcaaaactgtttaacagtgcatggaatgacagtacatggaatatagggaataataatacagggtcagataatgagacaatcattatcccatgcagaataaaacaaattataaacatgtggcagggagtaggaaaagcaatgtatgcccctcccatcgaaggatggatcaattgtgcatcaaatattacagggctcttactggtaagggatggtggtggtgcaaatgatagtcagaacgagaccttcagacctcaaggaggagatatgagagacaattggagaagtgaattatacaagtataaagtagtaaaaattgaaccactaggaatagcacccaccaaggcaaagagaagagtggtggaaagagaaaaaagagcaataggactaggagctatgttccttgggttcttgggagcagcaggaagcacgatgggcgcagcgtcattgacgctgacggtacaggccagacaattattgtctggtatagtgcaacatcaaaacaatttgctgatggctatagaggcgcaacagcatctgttgcaactcacagtctggggcattaaacagctccaggcaagaatcctggctgtggaaagatacctacaggatcaacagctcctaggaagttgggggtgctctggaagacacatttgcaccactactgtgccctggaactctagttggagtaataaatctatagatgacatttggaataacatgacctggatggagtgggaaaaagaaattgacaattacacaggtgtaatatacagattaattgaggaatcgcaaacccagcaagaaaagaatgaacaagaactattgcaattggacaaatgggcaagtttgtggaattggtttagcataacaaaatggctgtggtatataaaaatattcataatgatagtaggaggcttaatagggttaagaatagtttttactgtgctttctttagtaaatagagttaggcagggatactcacctctatcgtttcagaccctcttcccagccccgaggggacccgacaggcccgaagaaatagaagaaggaggtggagagcaaggcagaggcagatccactcgattggtgaacggattctcaacacttatctgggacgatctgaggaacctgtgcctcttcagctaccaccgcttgagagacttaatcttaattgcaacgaggattgtggaacttctgggacgcagggggtgggaagccatcaaatatttgtggaatctcctgcagtattggagtcaggaactgaagactagtgctattagcttgtttaacgctacagcagtagcagtagctgaggggacagatagggttatagaagtagtacaaagattttttagaggtattcttaacgtacccacacgaataagacagggcttggaaagggcgttactataa

SEQ ID NO: 15 - protein, 851 aa, HIV-1
MRVREIERNYQHLWRWITMLLGMLMICSVTGQLWVTVYYGVPVWKEATTTLFCASDAKAYKAEAHNIWATHACVPTDPNPQEIQLENVTENFNMWKNNMVEQMHEDIISLWDQSLKPCVKLTPLCVTLNCTEWENTNRTNNNVTNEEIGMKNCSFNTTTEVRDRKQQVHALFYKLDVVPMNDNNSTDINYTNYRLINCNTSAITQACPKVTFEPIPIHYCAPAGFAILKCNNKKFNGMGSCNNVSTVQCTHGIKPVVSTQLLLNGSLAEEEIIIRTENISDNAKIIIVQLNESVTINCTRPYNNTRKGTHIGPGRAWYTTGIVGDIRQAHCKVNKTEWNKTLERVAKKLRDLINKTTIKFSPPSGGDLEITTHSFNCGGEFFYCNTSRLFNSTWGDNNTSSDTEEGNITIPCRIKQIINMWQGVGKAMYAPPIEGLIRCSSNITGLLLTYDGGVNNNSQSEIFRPGGGDMRDNWRSELYKYKVVRLEPLGLAPTKAKRRVVEREKRAIGLGAMFLGFLGAAGSTMGAASLTLTVQARQLLSGIVQQQNNLLMAIEAQQHLLQLTVWGIKQLQARVLAVERYLKDQQLLGIWGCSGRLICTTNVPWNSSWSNKSIDEIWNNMTWMQWESEIDNYTGLIYKLIEESQIQQNKNEKELLELDKWASLWTWFDITNWLWYIKIFIMIVGGLIGLRIVFAVLSLVNRVRQGYSPLSFQTLLPVPRGPDRPEEIEEGGGEQDRGRSVRLVNGFSALIWDDLRNLCLFSYHRLRDLILIATRIVELLGRRGWETLKYLWNLLQYWIQELKNSAISLFNTTAIVVAEGTDRFLEIIQRIGRAILNIPTRIRQGFERALL

SEQ ID NO: 16 - DNA, 2556 bp, HIV-1
atgagagtgagggagatagagaggaattatcaacacttgtggagatggatcaccatgctccttgggatgttgatgatatgtagtgttacaggacagttatgggtcacagtttattatggggtacctgtgtggaaagaagcaaccactactctattttgtgcatcagatgctaaagcatataaagcagaggcacataatatctgggctacacatgcctgtgtaccaacagaccccaacccacaagaaatacaattagaaaatgtcacagaaaattttaacatgtggaaaaataacatggtggaacaaatgcatgaggatataatcagtttatgggatcaaagcctaaaaccatgtgtaaaattaaccccactctgtgtcactttaaactgcactgaatgggaaaatacaaatagaactaacaacaacgtcactaatgaggaaataggaatgaaaaactgctctttcaatacaaccacagaagtaagagataggaagcagcaagtacatgcacttttttataaacttgatgtggtaccaatgaatgataataatagtactgatatcaattataccaattatagactaataaattgtaatacctcagccattacacaggcgtgtccaaaggtaacctttgagccaattcccatacattattgtgccccagctggatttgcaattctaaagtgtaacaataagaagttcaatgggatgggatcatgcaacaatgtcagcacagtacagtgtacacatgggattaagccagtagtgtcaacccaattgttgttgaatggtagtctagcagaggaggaaataataattagaactgaaaatatctcagataatgcaaaaatcataatagtacagcttaatgagtctgtaacaattaattgcacaaggccctacaacaatacaagaaaaggtacacacataggaccagggcgagcatggtatacaacaggaatagtaggagatataagacaagcacactgtaaggttaataaaacagaatggaataaaactttagaacgggtagctaaaaaattaagagaccttattaataagacaacaataaagtttagtccaccctcgggaggggacctagaaattacaacacacagctttaattgtggaggggaatttttctactgcaatacatcaagactgtttaatagtacatggggggataataatacatcaagtgatacagaggaaggtaacatcaccatcccatgtagaataaaacaaattataaacatgtggcaaggagtaggaaaagcaatgtatgcccctcccattgaaggactaatcagatgttcatcaaatattacaggattactgttaacatatgatgggggtgtaaataataatagtcagagtgagatcttcagacctggaggaggagatatgagagacaattggagaagtgaattatacaaatataaagtagtaagacttgaaccactaggtctagcacccaccaaggcaaaaagaagagtggtggaaagagaaaaaagagcaataggcctaggagctatgttccttgggttcttgggagcagcaggaagcacgatgggcgcagcgtcattgacgctgacggtacaggccagacaattattgtctggtatagtgcagcagcaaaacaatctgctgatggctatagaggcgcaacagcatctgttgcaactcacagtctggggcattaaacagctccaggcaagagtcctggctgtggaaagatacctaaaggaccaacagctcctaggaatttggggttgctctggaagactcatttgcaccactaatgtgccatggaactctagctggagtaataaatccatagatgagatttggaataacatgacctggatgcagtgggaaagtgaaattgacaattacacaggtttaatatataaattaattgaagaatcgcaaatccagcaaaacaaaaatgaaaaagaactattggaattggacaaatgggcaagtttgtggacttggtttgacataacaaactggctgtggtatataaaaatattcataatgatagtaggaggcttgataggtttaagaatagtttttgctgtgctttctttagtaaatagagttaggcagggatattcacctctgtcttttcagaccctcctcccagtcccgaggggacccgacaggcccgaagaaatagaagaaggaggtggagagcaagacagaggcagatcagtgcgattggtgaacggattctcagcacttatctgggacgatctgaggaacctgtgcctcttcagctaccaccgcttgagagacttaatcttaattgcaacgaggattgtggaacttctgggacgcagggggtgggaaaccctcaaatatctgtggaatctcctgcagtactggattcaggaactaaagaatagtgctattagcttgtttaataccacagcaatagtagtagctgaggggacagataggtttttagaaataatacaaagaattggtagagctattcttaatatacccacgcgaataagacagggctttgaaagagctttactataa

SEQ ID NO: 17 - protein, 857 aa, HIV-1
MRVKETQMNWPNLWKWGTLILGLVIICSASDNLWVTVYYGVPVWKDADTTLFCASDAKAHETEVHNVWATHACVPTDPNPQEIHLENVTENFNMWKNNMVEQMQEDVISLWDQSLQPCVKLTPLCVTLHCTTAKLTNVTNITNVPNIGNITDEVRNCSFNMTTEIRDKKQKVHALFYKLDIVQIEDKNDSSKYRLINCNTSVIKQACPKISFDPIPIHYCTPAGYVILKCNDKNFNGTGPCKNVSSVQCTHGIKPVVSTQLLLNGSLAEEEIIIRSENLTNNAKTIIVHLNKSVEINCTRPSNNMRTSMRIGPGQVFYRTGSITGDIRKAYCEINGTKWNKVLKQVTEKLKEHFNNKTIIFQPPSGGDLEITMHHFNCRGEFFYCNTTQLFNNTCIGNETMKGCNGTITLPCKIKQIINMWQGTGQAMYAPPIDGKINCVSNITGILLTRDGGANNTSNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSFEEIWNNMTWIEWEREISNYTNQIYEILTESQNQQDRNEKDLLELDKWASLWNWFDITNWLWYIKIFIMIVGGLIGLRIIFAVLSIVNRVRQGYSPLSFQTPIHHQREPDRPERIEEGGGEQGRDRSVRLVSGFLSLAWDDLRSLCLFSYHRLRDFILIATRTVELLGHSSLKGLRRGWEGLKYLGNLLLYWGQELKISAISLLNTTAIAVAGWTDRVIEVAQGAWRAILHIPRRIRQGLERTLL

SEQ ID NO: 18 - DNA, 2574 bp, HIV-1
atgagagtgaaggagacacagatgaattggccaaacttgtggaaatgggggactttgatccttgggttggtgataatttgtagtgcctcagacaacttgtgggttacagtttattatggggttcctgtgtggaaagatgcagataccaccctattttgtgcatcagatgccaaagcacatgagacagaagtgcacaatgtctgggccacacatgcctgtgtacccacagaccccaacccacaagaaatacacctggaaaatgtaacagaaaattttaacatgtggaaaaataacatggtagagcagatgcaggaggatgtaatcagtttatgggatcaaagtctacagccatgtgtaaagttaactcctctctgcgttactttacattgtaccactgctaaattgaccaatgtcactaacataaccaatgtccctaacataggaaatataacagatgaagtaagaaactgttcttttaatatgaccacagaaataagagataagaagcagaaggtccatgcacttttttataagcttgatatagtacaaattgaagataagaatgatagtagtaagtataggttaataaattgtaatacttcagtcattaagcaggcttgtccaaagatatcctttgatccaattcctatacactattgtactccagctggttatgtgattttaaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacaaacaatgccaaaaccataatagtgcaccttaataaatctgtagaaatcaattgtaccagaccctccaacaatatgagaacaagtatgcgtataggaccaggacaagtattctatagaacaggaagcataacaggagatataagaaaagcatattgtgagattaatggaacaaaatggaataaagttttaaaacaggtaactgaaaaattaaaagagcactttaataataagacaataatctttcaaccaccctcaggaggagatctagaaattacaatgcatcattttaattgtagaggggaatttttctattgcaatacaacacaactgtttaataatacttgcataggaaatgaaaccatgaaggggtgtaatggcactatcacacttccatgcaagataaagcaaattataaacatgtggcagggaacaggacaagcaatgtatgctcctcccatcgatggaaaaattaattgtgtatcaaatattacaggaatactattgacaagagatggtggtgctaataatacgagtaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagccggaagcactatgggcgcggcgtcaataacgctgacggtacaggccagacaattattgtctggtatagtgcaacagcaaagcaatttgctgagggctatagaggcgcagcagcatctgttgcaactcacagtctggggcattaaacagctccaggcaagagtcctggctgtggaaagatacctaaaggatcaaaagttcctaggactttggggctgctctggaaaaatcatctgcaccactgctgtgccctggaactccacttggagtaataaatcttttgaagagatttggaacaacatgacatggatagaatgggagagagaaattagcaattacacaaaccaaatatatgagatacttacagaatcgcagaaccagcaggataggaatgaaaaggatttgttagaattggataaatgggcaagtctgtggaattggtttgacataacaaattggctgtggtatataaaaatatttataatgatagtaggaggtttaataggtttaagaataatttttgctgtgctttctatagtgaatagagttaggcagggatactcacctttgtctttccagacccctatccatcatcagagggaacccgacagacccgaaagaatcgaagaaggaggtggcgagcaaggcagagacagatccgtgcgattagtgagcggattcttatcacttgcctgggacgatctacggagcctgtgcctcttcagctaccaccgcttgagagacttcatcttgattgcaacgaggactgtggaacttctgggacacagcagtctcaagggactgagacgggggtgggaaggcctcaaatatctggggaatcttctgttatattggggccaggaactaaaaattagtgctatttctttgcttaatactacagcaatagcagtagcggggtggacagatagggttatagaagtagcacaaggagcttggagagccattctccacatacctagaagaatcagacagggcttagaaaggactttgctataa

SEQ ID NO: 19 - protein, 862 aa, HIV-1
MRVKETQMIWPNLWKWGTLILGLVIICSASDNLWVTVYYGVPVWRDAETTLFCASDAKAHETEVHNIWATHACVPTDPNPQEIRLENVTENFNMWKNNMVEQMQEDVISLWDQSLQPCVKLTPLCVTLNCTNATLNANLTYVNNITNGHNTIGNITDEVKNCSFKMTTELRDKRKKVHALFYKLDIVQLKGKGNKNKNNNFSQYRLMSCNTSVIKQACPKITFDPIPIHYCTPAGYAILKCNDKNFNGTGPCKDVSSVQCTHGIKPVVSTQLLLNGSLAEEEIVIRSENLTNNAKTIIVHLNKSVEINCTRPSNNTRASTRIGPGQVFYRTGDIIGDIRKAYCEINGTKWNEILKQVAEKLKEHFNNKTIIFQPPSGGDPEVTMHHFNCRGEFFYCNTSKMFNSTLGGLNGTIILPCKIKQIINMWQRVGQAVYAPPISGRINCVSNITGILLTRDGGANNATNETFRPGGGNIKDNWRSELYKYKVVQIEPLGIAPTRAKRRVVEREKRAAGIGAMIFGFLGAAGSTMGAASITLTVQARQLLSGIVQQQSNLLRAIEAQQHLLQLTVWGIKQLQARVLAVERYLNDQKFLGLWGCSGKIICTTAVPWNSTWSNKSYEEIWNNMTWVEWEREISNYTNQIYDILTESQNQQDKNEKDLLELDKWANLWSWFNITNWLWYIKIFIIIVGGLIGLRIVFAVLSIVNRVRQGYSPLSFQTPSHHQRETDRPEGIEEEGGEQGRDRSVRLVSGFLALVWDDLRSLCLFSYHRLRDFILIAARTLEILGHSSLKGLRRGWEVLKYLGNLLSYWGQELKTSAISLLDATAIAVAGWTDRVIEVARRAWRAFIHIPRRIRQGFERALL

SEQ ID NO: 20 - DNA, 2589 bp, HIV-1
atgagagtgaaggagacacagatgatttggccaaacttgtggaaatgggggactttgatccttggattggtaataatttgtagtgcctcagacaacttgtgggttacagtttattatggggttcctgtgtggagagatgcagaaaccaccctattttgtgcatcagatgccaaagcacatgagacagaagtgcacaatatttgggccacacatgcctgtgtacccacagaccccaacccacaagaaatacgtctggaaaatgtaacagagaattttaacatgtggaaaaataacatggtagagcagatgcaggaggatgtaatcagtttatgggatcaaagtctacagccatgtgtaaagttaactcctctctgcgttactttaaattgtaccaatgctacgttgaatgctaatttgacctatgtcaataacataactaatggccataatacaataggaaatataacagatgaagtaaaaaactgttcttttaagatgaccacagaactaagagataagaggaagaaagtccatgcacttttttataagcttgatatagtacaacttaaaggtaaaggtaataaaaataagaataataattttagtcagtataggttaatgagttgtaatacttcagtcattaagcaggcttgtccaaagataacctttgatccaattcctatacattattgtactccagctggttatgcgattttaaagtgtaatgataagaatttcaatgggacagggccctgtaaagatgtcagctcagtacaatgcacacatggaatcaagccagtggtatcaactcagttgttgttaaatggcagtctagcagaggaagagatagtaatcagatctgaaaatctcacaaacaatgccaaaaccataatagtgcaccttaataaatctgtagaaatcaattgtaccagaccctccaacaatacaagagcaagtacacgtataggaccaggacaagtattctatagaacaggagacataattggagatataaggaaagcatattgtgagattaatggaacaaaatggaatgaaattttaaaacaggtagctgaaaaattaaaagagcattttaataataagacaataatctttcaaccaccctcaggaggagatccagaagttacaatgcatcattttaattgtagaggggaatttttctattgcaatacatcaaaaatgtttaatagtactttgggggggcttaatggcactatcatacttccatgcaagataaagcaaataataaatatgtggcagagagtaggacaagcagtgtatgctcctcccatcagtggaagaattaattgtgtatcaaatattacaggaatactattgacaagagatggtggtgctaataatgcaactaacgagacctttagacctggaggaggaaatataaaggacaattggagaagtgaattatacaaatataaagtagtacaaattgaacctctaggaatagcacccaccagggcaaagagaagagtggtggagagagaaaaaagagcagcaggaataggagctatgatctttgggttcttgggagcagcaggaagcactatgggcgcagcgtcaataacgctgacggtacaggccagacaattattgtctggtatagtgcaacagcaaagcaatttgctgagagctatagaggcgcagcagcatctgttgcaactcacagtctggggcattaaacagctccaggcaagagtcctggctgtggaaagatacctaaatgatcaaaagttcctaggactttggggctgctctggaaaaatcatctgcaccactgctgtgccctggaactccacttggagtaataaatcttatgaagagatttggaacaacatgacatgggtagaatgggagagagaaattagcaattacacaaaccaaatatatgacatacttacagaatcacagaaccagcaggacaagaatgaaaaggatttgttggaattggataaatgggcaaatctgtggagttggtttaacataacaaactggctgtggtatataaaaatatttataataatagtaggaggcttaataggtttaagaatagtttttgctgtgctttctatagtaaatagagttaggcagggatactcacctttgtctttccagaccccctcccatcaccagagggaaaccgacagacccgaaggaatcgaagaagaaggtggcgagcaaggcagagacagatccgtgcgcttagtgagcggattcttagctcttgtctgggacgatctacggagcctgtgcctcttcagctaccaccgcttgagagacttcatcttgattgcagcgaggactctggaaattctgggacacagcagtctcaagggactgagacgggggtgggaagtcctcaaatatctggggaatcttctgtcatattggggccaggagctaaaaactagtgctatttctttgcttgatgctacagcaatagcagtagcggggtggacagatagggttatagaagtagcacgaagagcttggagagcttttatccacatacctaggagaatcagacagggctttgaaagggctttgctataa

SEQ ID NO: 21 - protein, 858 aa, HIV-1
MRVRGMQRSWQHLGKWGLLFLGILIICNATENLWVTVYYGVPVWKEATTTLFCASDAKAYESEVHNVWATHACVPTDPDPQEIKLNVTENFDMWKNNMVEQMHTDIISLWDQSLKPCVKLTPLCVTLDCTDVPINSTSTLKEDEGAIKNCSFNMTTEVRDKQQKVQALFYKLDMVPISDDSTRDSNVSSNGTRQYRLIHCNTSTITQACPKISWDPIPIHYCAPAGYAILKCYDTEFNGTGPCRNVSTVQCTHGIKPVVSTQLLLNGSLAGPEIIIRSQNISDNAKTIIVHLSESVWINCTRPNNNTRKSIHIGPGRAFHTTDRIVGDIRKAHCNVSRGEWSKTLGQVRKALGSHFPNGTTIKFNSSSGGDPEVTMHMFNCRGEFFYCNTSRLFNDTEFFNDTDSNSTDPITLPCRIRQIVNMWQEVGKAMYAAPIAGSITCNSTITGLLLTRDGGNNANKTENRTETFRPGGGNMKDNWRNELYKYKVVEIEPLGVAPTRAKRQVVKREKRAVGMLGAMFLGFLGAAGSTMGAASITLTVQARQLLSGIVQQQNNLLRAIEAQQHLLQLTVWGIKQLQARVLAVERYLQDQQFLGIWGCSGKLICTTTVPWNSSWSNKTYNEIWDKMTWMQWEKEISNYSTEIYRLIEESQYQQEKNEQELLSLDKWASLWNWFDISNWLWYIKIFIMIVGGLIGLRIVFAVLSIVNRVRKGYSPLSFQTHIPSPREPDRPEGIEEGGGEQDKDRSVRLVSGFLSLVWDDLRNLCLFSYRHLRDFILIAARIVDRGLKRGWEALKYLGNLTQYWGLELKNSAISLLNTTAIVVAEGTDRIIEALQRAGRAVLNIPRRIRQGLERALL

SEQ ID NO: 22 - DNA, 2577 bp, HIV-1
atgagagtgagggggatgcagaggagctggcagcacttggggaagtggggccttttattcctgggaatattaataatctgtaatgctacagaaaacttgtgggtcacagtctattatggggtacctgtgtggaaagaagcaaccactactctattctgtgcatcagatgctaaagcatatgaaagtgaggtgcataatgtctgggccacacatgcctgtgtacccacagatcccgatccacaagaaataaagctaaatgtaacagaaaattttgatatgtggaaaaataacatggtagaacaaatgcatacagatataattagtttatgggatcaaagcctaaagccatgtgtgaagttaaccccactctgtgttactttggattgtactgatgtccctatcaactccaccagcaccctgaaggaagacgaaggggcaataaaaaactgctctttcaatatgaccacagaagtaagagataaacagcagaaagtacaggcacttttttataaacttgatatggtaccaatcagtgatgacagtactcgtgatagcaatgtcagtagtaatggcactagacaatacaggctcatacattgtaatacttcaaccattacacaggcttgtccaaagatatcttgggatccaattcccatacattattgtgctccagctggttatgcgattctaaagtgttatgatacagaattcaatgggacggggccatgcaggaatgtcagcacagtgcaatgtacacatggaattaaaccagtggtatccactcaattgttgttaaatggcagcctagcaggaccagagataataatcaggtctcaaaatatttcagataatgcaaaaaccataatagtacatcttagtgagtctgtatggattaattgtacaagacccaataacaatacaagaaaaagtatacatataggaccaggacgtgcatttcatacaacagacagaatagtaggagacatcagaaaagcacattgtaacgttagtagaggagaatggagtaaaacattaggacaggtaaggaaagcgttagggtctcatttccctaatggaacaacaataaaatttaactcatcctcaggaggggacccagaagttacaatgcatatgtttaattgtagaggagaatttttctactgcaatacatcaagattgtttaatgacacagaatttttcaatgacacagattccaatagcactgaccctatcactctcccatgtcgaataagacaaattgtaaacatgtggcaggaagtaggaaaagcaatgtatgccgctcccattgcaggaagcattacctgtaactcaactattacaggcttactattgacaagagatggtggtaataatgctaataagactgaaaatcggactgaaaccttcagacctgggggaggaaatatgaaagacaattggagaaatgaattatataaatataaagtagtagaaattgaaccactaggagtagcacccaccagggcaaaaagacaagtggtgaagagagaaaaaagagcagtgggaatgctaggagctatgttccttgggttcttgggagcagcaggaagcactatgggcgcagcgtcaataacgctgacggtacaggccagacaattattgtctggaatagtgcaacagcagaacaatctgctgagggctattgaagcgcaacagcatctgttgcagctcacagtctggggcattaaacagctccaggcaagagtcctggctgtggaaagatacctacaggatcaacagttcctagggatttggggctgctctggaaaactcatctgcaccactactgtgccctggaactctagttggagtaataaaacttataatgaaatttgggataagatgacctggatgcaatgggaaaaggagattagcaattactcaacagaaatatacaggttaattgaagaatcgcagtaccagcaggaaaagaatgaacaagaattactgtcattggacaaatgggcaagtctgtggaattggtttgacatatcaaattggctatggtatataaaaatattcataatgatagtaggaggcttaataggcttaagaatagtttttgctgtgctttctatagtaaatagagttaggaagggatactcacctttgtcatttcagacccatatcccaagcccaagggaaccagacaggcccgaaggaatcgaagaaggaggtggagagcaagacaaagacagatccgtgagattagtgagcggattcttgagtcttgtctgggacgacctgcggaacctgtgcctcttcagctaccgccacttgagagacttcatattaattgcagcgaggattgtggacaggggactgaagagggggtgggaagccctcaaatacctggggaatctcacacagtattggggtctggaactaaagaatagtgctattagcttgcttaacaccacagcaatagtagtagctgaggggacagatagaattatagaagctttgcaaagagctggtagagctgttctcaacatacctagaagaataagacagggcttggaaagagctttgctttaa

SEQ ID NO: 23 - protein, 849 aa, HIV-1
MRVREMQRNWQHLGKWGLLFLGILIICNAADNLWVTVYYGVPVWKEATTTLFCASDAKAYEREIHNVWATYACVPTDPNPQELVLGNVTENFNMWKNNMVDQMHEDIISLWDQSLKPCVQITPLCVTLNCTDVPVNITNGNSTLDNITLEEQGEIKNCSFNITTEINDIKKKESAIFYRLDVVPINNSTSEYRLLSCNTSTVTQACPKVSFDPIPIHYCAPAGFAILKCNDKEFNGTGLCRNVSTVQCTHGIKPVVSTQLLLNGSLAEGDIVIRSENISDNAKTIIVQFNRSVAINCTRPTNITRRSMRIGPGRVFYATGTVLGDIRKAYCTINGTLWNKTLEGVAKEVQSHLNKSITFAPSSGGDLEVTTHSFNCRGEFFYCNTVALFNATNMTNAMNRSNGIITLPCRIRQIVNMWQRVGRAMYAAPIAGQIQCNSSITGLILTRDGGKNNTNNDTLRPGGGDMRDNWRSELYKYKVVKIEPLGVAPTKAKRQVVKREREKRAVGIGAVLLGFLGAAGSTMGAASMTLTVQARQLLSGIVQQQNNLLKAIEAQQHLLQLTVWGIKQLQARILAVERYLKDQQLLGIWGCSGKLICTTNVPWNSSWSNKSQNEIWENMTWMQWEKEISNYTGTIYKLIENAQNQQEKNEQDLLALDKWDNLWSWFTITNWLWYIKLFIMIVGGLIGLRIVFAVLAVINRVRQGYSPLSLQTLTPSRREPERPGGIEEEGGEQDKTRSVRLVSGFLALAWDDLRSLCLFSYRHLRDFILIAARTVNKGLIRGWEILKYLGNLAQYWGREIKNSAIDLLNTTAIVVAEGTDRIIEVLQRAGRAILHIPRRIRQGAERALL

SEQ ID NO: 24 - DNA, 2550 bp, HIV-1
atgagagtgagggagatgcagaggaattggcagcacttggggaaatggggccttttattcctggggatattaataatctgtaatgctgcagataacttgtgggtcacagtctattatggagtacctgtgtggaaagaagcaaccactactctattttgtgcatcagatgctaaagcatatgaaagagagatacataatgtctgggctacatatgcctgtgtacctacagaccccaacccacaagaattagttctgggaaatgtaacagaaaattttaacatgtggaaaaataacatggtagaccagatgcatgaagatataatcagtttatgggatcaaagcctaaagccatgtgtacagataaccccactctgtgtaaccttaaattgtactgatgttcctgttaacatcactaatgggaacagcaccctggataacatcaccctggaagaacaaggggaaataaaaaactgttctttcaatatcaccacagagataaacgatattaagaaaaaagaatctgcaattttttataggcttgatgtagtaccaatcaataatagtactagtgaatataggctactaagttgtaatacctcaaccgttacacaggcttgtccaaaggtgtcctttgatccaattccaatacattattgtgctcctgctggttttgcgattctaaagtgtaatgataaagagttcaatgggacagggttatgtaggaatgtcagcacagtacaatgtacacatggaattaaaccagtagtgtcaactcaactactgttaaatggcagcctagcagaaggagatatagtaattagatctgaaaatatctcagataatgcaaaaaccataatagtacagtttaatagatctgtagcaattaactgtacaagacccaccaacattacaagaagaagtatgcgtataggaccaggacgagtattttatgcaacaggtaccgtactaggagatataagaaaggcatattgtaccattaatggaacactgtggaataaaactttagaaggagtagctaaagaggtccaaagccaccttaataaatcaataacatttgcgccatcatcaggaggggacctagaagttacaacacatagttttaattgtagaggagagtttttctactgcaacacagtagctctgtttaatgcaactaacatgactaatgcaatgaacaggtccaatggcattatcactcttccatgtagaataagacaaattgtaaacatgtggcaaagagtaggacgagcaatgtatgccgctcccattgctggacaaattcagtgtaactcaagcatcacaggtctaatattgacaagagatggtgggaaaaataataccaataatgacaccctcagacctggagggggagatatgagagacaattggagaagtgaactgtataaatataaggtagtaaaaattgaaccactaggagtagcacccaccaaggcaaaaagacaagtggtgaagagagaaagagaaaaaagagcagtgggaataggagctgtgctccttgggttcttgggagcagcaggaagcactatgggcgcggcgtcaatgacgctgacggtacaggccagacaattattgtctggtatagtgcaacagcaaaacaatttgctgaaggctatagaagcgcaacagcatctgttgcagctcacagtctggggcattaaacagctccaggcgagaatcctggctgtggaaagatacctaaaggaccaacagctcctagggatttggggatgctctggaaaactcatctgcaccactaatgtgccctggaattctagttggagtaataaatctcagaatgaaatttgggagaacatgacctggatgcagtgggaaaaagagatcagtaattacacaggcacaatatacaaattaatagaaaatgcacaaaaccagcaggaaaagaatgaacaggacttattggcattggacaaatgggacaatctgtggagttggtttactataacaaattggttgtggtacataaaattattcataatgatagtaggaggcttgataggattaagaatagtttttgctgtgcttgctgtaataaatagggttaggcagggatactcacctttgtcgttacagacccttaccccaagccggagggaacccgaacggcccggaggaatcgaagaagaaggtggagagcaagacaaaaccagatccgtcagattagtgagcggattcttagcacttgcctgggacgacctacggagcctgtgcctcttcagctaccgccacttgagagacttcatattaattgcagcgaggactgtgaacaagggactaataagggggtgggaaatcctcaaatatctggggaatctcgcgcagtattggggccgggaaataaagaatagtgctattgatctgcttaataccacagcaatagtggtagctgaagggacagatagaatcatagaagttctgcaaagagctggtagagctattctccacatacctagaagaataagacagggtgcagaaagggctttgctataa

SEQ ID NO: 25 - protein, 863 aa, HIV-1
MRVKGIQTNWQHLWKWGTLILGLVIICSASDKLWVTVYYGVPVWEDADTTLFCASDAKSYSSEKHNVWATHACVPTDPNPQEIVMKNVTEYFNMWKNNMVEQMHEDIISLWDESLKPCVKLTPLCVTLNCTDIVYSNCTRKHPNGTVDYNSTVDNSSCEIIKNCSFNITTELRDKSKKEYALFYRPDVVPIDGNDNSTYSDYRLINCNVSSIKQACPKVIFDPIPIHYCAPAGFAILKCGDKKVQIGTGTYVTNVSTVQCTHGIKPVVSTQLLLNGSVTEEEIIIQSENVTDNSKVIIVQFNDTVEINCTRPGNNTRRSIRFGPGQAFYATGDIIGNIRQAHCIVNGEKWNQVIQKVKTHLEEIYNKTIIFSSSAGGDLEITTHSFNCRGEFFYCNTSRLFNNETGNSTNTNIHSHVNKQIVECGREWDKQCMPLPSKEKLDVTSTITGLILPRDGGNSTNTNNTETFRPGGGDMRDNWRSELYKYKTVKIKSLGIAPTRARRRVVEREKRAVGLGAVFLGFLGAAGSTMGAASITLTVQVRQLLYGIVQQQSNLLRAIEAQQHMLQLTVWGIKQLQARVLAVERYLRDQQLLGIWGCSGKLICTTNVPWNTSWSNKTYNEIWDNMTWIQWEKEINNYTKQIYSLIEESQNQQEKNEQELLTLDKWASLWSWFDITKWLWYIKIFIMIVGGLIGLRIVFTVLSIVNRVRQGYSPLSFQTLTHQQREPDRLERTEEGGGEQDRDRSVRLVSGFLALAWDDLRSLCLFSYHRLRDFALIAARTVELLGRSSLKGLRLGWEGLKYLWNLLLYWVQELKNSAISLLDTVAIAVANWTDRVIEVIQRAGRAILNIPTRIRQGFERALL

SEQ ID NO: 26 - DNA, 2592 bp, HIV-1
atgagagtgaaggggatacagacgaattggcagcacttgtggaaatgggggactttgatccttgggttggtgataatctgtagtgcctcagacaaattgtgggtcacagtctattatggggtacctgtgtgggaagatgcagataccactctattctgtgcatctgatgctaaatcatatagttctgaaaaacataatgtctgggctacacatgcctgtgtacccacagaccccaatccgcaagaaatagttatgaaaaatgtaactgaatattttaacatgtggaaaaataacatggtagaacagatgcatgaagatataatcagtctatgggatgaaagcctaaagccatgtgtaaagctaacccctctctgtgttactctaaactgtactgacattgtctattctaattgtactagaaagcatcccaatggcactgtggattacaatagcactgtggataacagtagctgtgaaataataaaaaactgctctttcaatataactacagaactaagagacaaaagtaagaaagagtacgcgctcttctataggcctgatgtagtaccaattgatggtaatgataatagtacttatagtgattataggctaataaattgtaatgtctcatctatcaagcaggcttgtccaaaggtaatttttgacccaattcccatacactattgtgctccagctggttttgcgattttaaaatgtggggataagaaagttcaaattggaacagggacttatgtaaccaatgttagtacagtacaatgcacacatggaattaagccagtggtatcaacccaactactgctgaatggcagtgtgacagaggaagaaataataattcaatctgaaaatgtcacagacaatagtaaagtcataatagtacagtttaatgatactgtagaaattaattgtaccagacccggcaacaatacaagaagaagtataagattcggaccaggacaagcattctatgcaacaggtgacataataggaaacataagacaagcacattgtattgttaatggagaaaaatggaatcaggtgatacaaaaggtcaaaacacatctagaggaaatctataataagaccataatctttagctcatccgcaggaggggacctagaaattacaacacatagtttcaattgtagaggagaattcttctattgtaatacatcaaggctgtttaataatgaaactgggaatagcacaaacacaaatatccactcccatgtaaataaacaaattgtagaatgtggcagagagtgggacaagcaatgtatgcccctcccatcgaaggagaaattagatgtgacctcaacaattacaggactaatattaccaagagatggtgggaacagtaccaatactaataatacagagaccttcagacctggaggaggagatatgagggacaattggagaagtgaattatataagtacaaaacagtaaaaatcaaatcactaggaatagcacccaccagggcaaggagacgagtggtggagagagaaaaaagagcagttggactgggagctgtcttccttgggttcttaggagcagcaggaagcactatgggcgcggcgtcaataacgctgacggtacaggtcaggcaattattgtacggcatagtgcaacagcaaagcaatttgctgagggctatagaggcgcaacagcatatgttgcaactcacagtctggggcattaaacagctccaggcaagagtcctagctgtggaaagatacctaagggatcaacagctcctagggatttggggctgctctggaaaactcatctgcaccactaatgtaccctggaacactagttggagtaataaaacttataatgagatttgggataacatgacttggatacaatgggaaaaggaaattaacaattacacaaaacaaatatacagcctaattgaagaatcgcagaaccagcaggaaaagaatgaacaagaattattaacattggacaaatgggcaagtttgtggagttggtttgacataacaaaatggctatggtatataaaaatattcataatgatagtaggaggtttaataggtttaagaatagtttttactgtgctttctatagtaaatagagttaggcaggggtactcacctttgtcattccagacccttacccatcagcagagggaaccagacaggctcgaaagaaccgaagaaggaggtggcgagcaagacagagacaggtccgtgcgcttagtgagcggtttcttagcgcttgcctgggacgacctacggagcctgtgcctcttcagctaccaccgcttgagagactttgccttgattgcagcgaggacagtggaacttctgggacgcagcagtctcaagggactgagactggggtgggaaggcctcaaatatctgtggaatctcctgttgtattgggttcaggaactaaagaatagtgctattagtttgcttgatacagtagcaatagcagtagctaactggacagatagggttatagaagtaatacaaagagctggcagagctattcttaatatacctacaaggataagacaaggctttgaaagagctttgctataa

SEQ ID NO: 27 - protein, 852 aa, HIV-1
  VARIANT (0)...(0): Xaa = any amino acid
METQRNYPHLLSGGILILGMLLMCSTGENLWVTVYYGVPVWKEAKTTLFCASDAKAYSTEKHNVWATHACVPTDPSPQEMVLENVTENFNMWENNMVDQMHEDIISLWDQSLKPCVKLTPLCVTLECGEVGNKTSNSNSSASLEGEQKLTNCSFNVTTAIRDRKKKVQALFYRIDVVPINDEDYNGTNGTNRTLYRLINCNTSVITQACPKVTFEPIPIHYCAPAGFAILKCNNKTFNGTGPCTNVSTVQCTHGIKPVVSTQLLLNGSLAEGQVIIRSKNITDNTKNIIVQFNESVQINCTRPNNNTRKGIHIGPGQAFYVTGEVIGNIRQAHCNISGTQWNKTLYNVVNQLRKHFNKTIIFEPSSGGDIEITSHTFNCGGEFFYCNTSRLFNSTWNSNDTGNGTDNSTITLPCKIKQIINMWQRVGQAMYAPPIQGNITCVSNITGLILTLDRYVDNGTNVTLRPGGGDMRDNWRSELYKYKVVQIEPLGIAPTKARRRVVEREKRAVGMGAFFLGFLGAAGSTMGAASITLTVQARQLLSGIVQQQSNLLRAIQAQQHMLQLTVWGVKQLQARVLAVERYLKDQQLLGIWGCSGKLICTTNVPWNSSWSNKSYXXIWXNMTWMEWERQIDNYTXEIYXLLEISQNQQEKNEQDLLSLDKWSSLWNWFDISXWLWYIKIFIMVIGGLIGLRIVFTVLSIINSVRQGYSPLSFQTLTPNARRPDRPEGIEEEGGEQGRDRSTPLVSGFFILVWQDLWNLCLFSYRRLRDLLLIVARTVELLGRRGWEALKYLWNLLQYWGQELKNSAVNLLNTTAIVVAEGTDRIIELVQRAGRAIIHIPRRIRQGFERALL

SEQ ID NO: 28 - protein, 790 aa, HIV-1
  VARIANT (0)...(0): Xaa = any amino acid
METQKNWQTLWRGGLMIFGMLMICKAKEDLWVTVYYGVPVWKDAKTTLFCASDAKAYSTEKHNVWATHACVPTDPSPQEMNLPNVTENFNMWKNDMVDQMQEDIISVWDESLKPCVKITPLCVTLNCSNITSNSNTTSNSSVSSPDIMTNCSFNITTEIRNKRKQEYALFYRQDVVPIDSNNKNYILINCNTSVIKQACPKVSFQPIPIHYCAPAGFAILKCNDKNFNGTGSCKNVSTVQCTHGIKPVVSTQLLLNGSIAEGDIIIRSENISDNAKNIIVQLNKTVEIVCYRPNNNTRKGIHMGPGQVLYATGEIIGNIRETHCNISERDWSNTLRRVATKLREHFNKTINFTSPSGGDIEIVTHSFNCGGEFLYCNTSKLFNSSWDKNSIEATNDTSXATITIPCKIKQIVRMWQRTGQAIYAPPIAGNITCTSNITGLLLTRDGGNRGNGSENGTETFRPTGGNMKDNWRSELYKYKVVEIEPLGVAPTKAKRRVVEREKRAVGIGAVFLGFLGTAGSTMGAASITLTVQVRQLLSGIVQQQSNLLKAIEAQQHLLKLTVWGIKQLQARVLAVERYLKDQQLLGIWGCSGKLICTTNVPWNASWSNKSYEDIWENMTWIQWEGLIGLRIIFAVLAIVNRVRQGYSPLSFQTLIPNPTEADRPGGIEEGGGEQGRTRSIRLVNGFLALAWDDLRNLCLFSYHRLRDFVLIAARTVGTLGLRGWEILKYLVNLVWYWGQELKNSAISLLNTTAIAVAEGTDRIIEIAQRAFRAILHIPRRIRQGLERALL

SEQ ID NO: 29 - protein, 842 aa, HIV-1
MRVRGMQRNWQTLGNWGILFLGILIICSNADKLWVTVYYGVPVWKEATPTLFCASDAKAYEKEVHNVWATHACVPTDPNPQEVEMENVTENFNMWKNNMVEQMHTDIISLWDESLKPCVELTPLCVTLNCTDYKGTNSTNNATSTVVSPAEIKNCSFNITTEIKDKKKKESALFYRLDVLPLNGEGNNSSTEYRLINCNTSTITQTCPKVTFEPIPIHYCAPAGFAILKCKDKRFNGTGPCKNVSTVQCTHGIKPVVSTQLLLNGSLAEEEIIIRSENITDNTKNIIVQLNETVQINCTRPNNNTRKSIHMGPGKAFYTTGDIIGDIRQAHCNISGEKWNMTLSRVKEKLKEHFKNGTITFKPPNPGGDPEILTHMFNCAGEFFYCNTTKLFNETGENGTITLPCRIKQIINMWQKVGKAIYAPPIAGSINCSSNITGMILTRDGGNNTHNETFRPGGGDMRDNWRSELYKYKVVQIEPLGIAPTRARRRVVQREKRAVGLGAVFFGFLGAAGSTMGAASITLTVQARQLLSGIVQQQSNLLRAIEAQQHLLQLTVWGIKQLRARILAVERYLKDQQLLGIWGCSGKLICTTNVPWNSSWSNKSWEEIWNNMTWMEWEKEIGNYSDTIYKLIEESQTQQEKNEQDLLALDKWASLWNWFDITKWLWYIKIFIMIIGGLIGLRIAFAVLSVVNRVRQGYSPLSFQTLIPTSRGADRPEGIEEEGGEQDKNRSVRLVSGFLALAWDDLRNLCLFSYRQLRNLILIVTRILERGLRGGWEALKYLWNLVQYWSQELKNSAISLLNTTAIAVAGGTDRIIEIGQRAFRALLHIPRRIRQGLERALL

SEQ ID NO: 30 - DNA, 2553 bp, HIV-1
gagaaagagcagaagacagtggcaatgagagtgagggggatgcagaggaattggcagacattggggaactggggcatcttattcttgggaatattgataatctgtagcaatgcagacaaattgtgggtcacagtctattatggggtacctgtgtggaaagaagcaacacccactctattctgtgcatcagatgctaaagcatatgagaaggaggtacataatgtctgggctacccatgcctgtgtacccacggaccccaacccacaagaagtagagatggaaaatgtaacagaaaactttaacatgtggaaaaataacatggtagagcagatgcatacagatataatcagtttatgggatgaaagcctaaaaccttgtgtagagttaacccctctctgtgtcactttaaattgtactgattacaaaggaaccaatagcaccaataatgcaactagcactgtggtaagcccagcagaaataaaaaattgctctttcaatataaccacagaaataaaagataagaagaaaaaggaatctgcacttttctatagacttgatgtactgccacttaatggcgagggtaataacagtagtactgaatacaggctaataaattgtaatacctcaaccattacacagacttgtccaaaagtaacctttgagccaattcccatacattattgtgccccagctggctttgcgattctaaagtgtaaggataaaaggttcaatgggacaggaccatgtaaaaatgtcagcacagtacaatgtacacatggaattaaaccagtggtatcaactcaactgctgttaaatggcagcctagcagaagaagagataataattaggtctgaaaatattacagataatacaaaaaacataatagtacagcttaatgaaactgtacaaattaattgtacaaggccaaacaacaatacaagaaaaagtatacatatgggaccaggaaaagcattctatacaacaggtgatataataggagatataagacaggcacattgcaacattagtggagaaaaatggaacatgactttaagcagagtaaaggaaaagctaaaagaacattttaagaatggaacaataacatttaaaccaccaaacccaggaggagacccagaaattctaacgcacatgtttaattgtgcaggagaatttttctactgcaatacaacaaaactgtttaatgagacaggggagaatggtactatcacactcccatgtagaataaagcagattataaacatgtggcagaaagtgggaaaagcaatatatgcccctcccattgcaggaagtattaactgtagctcaaatattacaggaatgatattgacaagagatggtggtaataatactcataatgagaccttcagacctggaggaggagacatgagggacaattggagaagtgaactgtataaatataaagtagtacagattgaaccactaggaatagcacccaccagggccaggagaagagtggtgcagagagaaaaaagagcagtaggattaggagctgtgttctttggattcttgggagcagcaggaagcactatgggcgcggcgtcaataacgctgacggtacaggccagacaattattgtctggtatagtgcaacagcaaagcaatttgctgagagctatagaagcgcaacaacatctgttacagctcacggtctggggcattaaacagctccgggcaagaatcctggctgtagaaagatacctaaaggatcaacagctcctagggatttggggctgctctggaaaactcatctgcaccactaatgtgccctggaactctagctggagtaataaatcttgggaagagatttggaacaacatgacctggatggagtgggaaaaagagattggcaattactcagacacaatatataagttaattgaagaatcacaaacccagcaggaaaagaatgaacaagatttattggcattggacaaatgggcaagtctgtggaattggtttgacataacaaaatggctatggtatataaaaatattcataatgataataggaggcttgataggtttaagaatagcttttgctgtgctttctgtagtaaatagagtcaggcagggatactcacctttgtcatttcagacccttatcccaacctcgaggggagcagacagacccgaaggaatcgaagaagaaggtggagagcaagacaaaaacagatcagttcgattagtgagcggcttcttagcgcttgcctgggacgatctgcggaacctgtgcctcttcagctaccgccaattgagaaacttaatcttaattgtgacgaggatcctggaaaggggactgagggggggttgggaagccctcaaatatctgtggaaccttgtacagtattggagtcaggaactaaagaatagtgccattagcttgcttaataccacagcaatagcagtagctggaggaacagatagaattatagaaataggacaaagagcttttagagctttacttcacatacctagaagaataagacagggtctcgaaagagctttactataa

SEQ ID NO: 31 - protein, 842 aa, HIV-1
MGMKSGWLLFYLLVSLIKVIGSEQHWVTVYYGVPVWREAETTLFCASDAKAHSTEAHNIWATQACVPTDPNPQEVLLPNVTEKFNMWENKMADQMQEDIISLWEQSLKPCVKLTPLCVTMLCNDSYGEERNNTNMTTREPDIGYKQMKNCSFNATTELTDKKKQVYSLFYVEDVVPINAYNKTYRLINCNTTAVTQACPKTSFEPIPIHYCAPPGFAIMKCNEGNFSGNGSCTNVSTVQCTHGIKPVISTQLILNGSLNTDGIVIRNDSHSNLLVQWNETVPINCTRPGNNTGGQVQIGPAMTFYNIEKIVGDIRQAYCNVSKELWEPMWNRTREEIKKILGKNNITFRARERNEGDLEVTHLMFNCRGEFFYCNTSKLFNEELLNETGEPITLPCRIRQIVNLWTRVGKGIYAPPIRGVLNCTSNITGLVLEYSGGPDTKETIVYPSGGNMVNLWRQELYKYKVVSIEPIGVAPGKAKRRTVSREKRAAFGLGALFLGFLGAAGSTMGAASITLTVQARTLLSGIVQQQNILLRAIEAQQHLLQLSIWGIKQLQAKVLAIERYLRDQQILSLWGCSGKTICYTTVPWNETWSNNTSYDTIWNNLTWQQWDEKVRNYSGVIFGLIEQAQEQQNTNEKSLLELDQWDSLWSWFGITKWLWYIKIAIMIVAGIVGIRIISIVITIIARVRQGYSPLSLQTLIPTARGPDRPEETEGGVGEQDRGRSVRLVSGFSALVWEDLRNLLIFLYHRLTDSLLILRRTLELLGQSLSRGLQLLNELRTHLWGILAYWGKELRDSAISLLNTTAIVVAEGTDRIIELAQRIGRGILHIPRRIRQGLERALI

SEQ ID NO: 32 - DNA, 2529 bp, HIV-1
atggggatgaagagtggttggttactcttctatcttctagtaagcttgatcaaggtaattgggtctgaacaacattgggtaacagtgtactatggggtaccagtatggagagaagcagagacaactcttttctgtgcttcagatgctaaagcccatagtacagaggctcacaacatctgggccacacaagcatgtgttcctactgatcccaatccacaagaagtgctattacccaatgtaactgaaaaatttaatatgtgggaaaataaaatggcagaccaaatgcaagaggatattatcagtctgtgggaacagagcttaaagccctgtgttaaattaaccccattatgtgtaactatgctttgtaacgatagctatggggaggaaaggaacaatacaaatatgacaacaagagaaccagacataggatacaaacaaatgaaaaattgctcattcaatgcaaccactgagctaacagataaaaagaagcaagtttactctctgttttatgtagaagatgtagtaccaatcaatgcctataataaaacatataggctaataaattgtaataccacagctgtgacacaagcttgtcctaagacttcctttgagccaattccaatacattactgtgcaccaccaggctttgccattatgaaatgtaatgaaggaaactttagtggaaatggaagctgtacaaatgtgagtactgtacaatgcacacatggaataaagccagtgatatccactcagttaatcctaaatggaagcttaaatacagatggaattgttattagaaatgatagtcacagtaatctgttggtgcaatggaatgagacagtgccaataaattgtacaaggccaggaaataatacaggaggacaggtgcagataggacctgctatgacattttataacatagaaaaaatagtaggagacattagacaagcatactgtaatgtctctaaagaactatgggaaccaatgtggaatagaacaagagaggaaataaagaaaatcctggggaaaaacaacataaccttcagggctcgagagaggaatgaaggagacctagaagtgacacacttaatgttcaattgtagaggagagtttttctattgtaacacttccaaattatttaatgaggaattacttaacgagacaggtgagcctattactctgccttgtagaataagacagattgtaaatttgtggacaagggtaggaaaaggaatttatgcaccaccaattcggggagttcttaactgtacctccaatattactggactggttctagaatatagtggtgggcctgacaccaaggaaacaatagtatatccctcaggaggaaacatggttaatctctggagacaagagttgtataagtacaaagtagttagcatagaacccataggagtagcaccaggtaaagctaaaagacgcacagtgagtagagaaaaaagagcagcctttggactaggtgcgctgtttcttgggtttcttggagcagcagggagcactatgggcgcagcgtcaataacgctgacggtacaggcccggacattattatctgggatagtgcaacagcagaatattctgttgagagcaatagaggcgcaacaacatttgttgcaactctcaatctggggcattaaacagctccaggcaaaagtccttgctatagaaagataccttagggatcagcaaatcctaagtctatggggctgctcaggaaaaacaatatgctataccactgtgccttggaatgagacttggagcaacaatacctcttatgatacaatctggaataatttaacctggcaacaatgggatgagaaagtaagaaactattcaggtgtcatttttggacttatagaacaggcacaagaacaacagaacacaaatgagaaatcactcttggaattggatcaatgggacagtctgtggagctggtttggtattacaaaatggctgtggtatataaaaatagctataatgatagtagcaggcattgtaggcataagaatcataagtatagtaataactataatagcaagagttaggcagggatattctcccctttcgttgcagacccttatcccaacagcaaggggaccagacaggccagaagaaacagaaggaggcgttggagagcaagacagaggcagatccgtgcgattagtgagcggattctcagctcttgtctgggaggacctccggaacctgttgatcttcctctaccaccgcttgacagactcactcttgatactgaggaggactctggaactcctgggacagagtctcagcaggggactgcaactactgaatgaactcagaacacacttgtggggaatacttgcatattggggaaaagagttaagggatagtgctatcagcttgcttaatacaacagctattgtagtagcagaaggaacagataggattatagaattagcacaaagaataggaaggggaatattacacatacctagaagaatcagacaaggcctagaaagagcactgatataa

SEQ ID NO: 33 - protein, 878 aa, HIV-1
MTVMEKKSKKSWILCIAMALIIPCLSGRQLYITVYSGVPVWEDATPVLFCASDANLTSTEKHNVWASQACVPTDPTPHEYPLVNVTDKFDIWKNYMVDQMHEDIISLWDQSLKPCVQMTFLCVQMNCTELSNSSASNTTDLRIAGLEEIPMKNCSFNVTTFLNDRKEKRQALFYVSDLVKIDNSSTIYRLTNCNSTTIRQACPKVSFEPIPIHYCAPAGYAIFKCNNTDFNGTGLCPNISVVTCTHGIKPTVSTQLIMNGTLSRGKIRIMGRNITDNTKNIVVTLNTSINMTCTRKGRGKIQRIATGPLRWVSMAAKTESQNTGSRIAYCMYNNTEWINTLKQTAERYLELVNKTRNLSKTRNFSIIFNHSISGGDIEASSLHFNCHGEFFYCDTSRLFNYTFKCNGSQCNETNKTQTNKTQDTIIPCKIRQVVRSWIKGELGLYAPPIPGDLTCKSNITGMILQLDTPYNSSCDNVTFRPTGGDMRDIWRTELYNYKVIQVKPFSVAPTKISRPIIGLNTTHRGKRAVGLGMLFLGVLSAAGSTMGAAATTLAVRTQGVLKGIVQQQDNLLRAIQAQQHLLRLSVWGIRQLRARLQALETLIQNQQRLSLWGCKGRIICYTSAKWNNTWGNWTDSAWNNLTWQQWDQQIDEYSTTIYTKIQEAQDQQEQNEKTLLELDEWASLWNWFDITKWLWYIKIAIIIVGALIGIRVVMIVLNLVKNIRQGYQPLSLQIPIPHQEEAETPGRTGEEGGGVDRRKWTPLQPGFLQLLYTDLRTIILWTYHLLSNLASGIQRLISYLGLGLWILGQKTIEACRLCGAVTQYWLQELRASATNLLDTIAVAVGNWTDSIILGIQRIGRGFLNIPRRIRQGAERALN

SEQ ID NO: 34 - DNA, 2637 bp, HIV-1
atgacagtaatggagaagaagagcaagaagtcatggatcttatgcatagccatggctttgataatcccatgtttgagtggtagacaattgtatatcacagtctattctggggtacctgtatgggaagatgcaacaccagtactattctgtgcttcagatgctaatttgacaagcactgaaaagcataatgtttgggcatcacaagcctgcgttcccacagaccccactccacatgaatatccactagtcaatgtgacagataaatttgatatatggaaaaattacatggtggaccaaatgcatgaagacattattagtttatgggatcaaagtttaaagccttgtgtgcaaatgactttcttatgtgtacaaatgaactgtacagagctgagtaatagcagtgcatcaaacacaaccgacctaaggatcgcaggcctagaagaaatacccatgaaaaattgtagttttaatgtaactacattcctcaatgacagaaaggagaaaaggcaggctctattctatgtatcagatttggttaagattgacaacagctcaacaatatatagattaactaattgtaattccacaaccatcagacaagcctgtccgaaggtaagctttgagcccatccccatacattattgtgctccagcaggatatgccatctttaagtgtaataacacagactttaatggaacaggcctatgtcccaatatttcagtggttacatgtacacatggcatcaagccaacagtaagtactcaattaataatgaatgggacactctctagagggaagataagaattatgggaagaaatattacagacaatacaaagaatattgtagtaaccctaaacacttctataaacatgacatgtacgagaaaaggaagaggtaaaatacaaaggatagcgacaggtccactgcgatgggtcagtatggcagctaaaacagagtcacagaacacagggtcaaggatagcttattgtatgtataacaacactgaatggataaataccttaaaacaaacagctgaaagatatttagaactagtaaacaagacaagaaatttaagcaagacaagaaattttagcataatattcaaccacagtataagtggtggagacatagaagcaagctctttacattttaactgtcatggagaattcttttattgtgacacatctcggctgtttaactatacttttaagtgtaatggttcccaatgtaatgagaccaataaaactcagacaaataaaactcaggatactataataccttgcaagataagacaggtagtaagatcatggataaagggagagttaggactctatgcacctcccatcccaggtgatctaacatgtaaatccaacataactgggatgattttacaactagatacaccctacaactcctcatgtgacaatgtcacatttagaccaacagggggagatatgagagatatatggagaactgaattgtacaactacaaagtaatacaggtaaaaccttttagtgtagcacctacaaaaatttcaagaccaataataggccttaacaccacccacagaggaaaaagagcagtaggattgggaatgctattcttaggggttctaagcgcagcaggtagcactatgggcgcagcggcaacaacgctggcggtacggacccaaggtgtactaaagggtatagtgcaacagcaggacaacctgctgagagcgatacaggcccagcaacatttgctgaggttatctgtatggggtattagacaactccgagctcgcctgcaagccttagaaacccttatacagaatcagcaacgcctaagcctatggggatgtaaaggaaggataatatgttacacatcagcaaaatggaacaacacatggggaaactggactgacagtgcttggaacaacttgacatggcagcaatgggaccaacaaatagatgaatatagcaccactatatacactaaaatacaagaagcacaggaccaacaggaacagaatgaaaagacattgttagagctagatgaatgggcttctctttggaattggtttgacataactaaatggttgtggtatataaaaatagctataatcatagtaggagcactaataggtataagagttgtcatgatagtacttaatctagtgaaaaacattaggcagggatatcaacccctctcgttgcagatccccatcccacaccaggaggaagcagaaacgccaggaagaacaggagaagaaggtggaggcgtagacaggcgcaagtggacacccttgcaaccaggattcttacaactgttgtacacggatctcaggacaataatcttgtggacttaccacctcttgagcaacttagcatcagggatccagaggttgatcagctacctgggacttggactgtggatcctgggacaaaagacaattgaagcttgcagactttgtggagctgtaacacaatactggttacaagaattgcgggctagtgctacaaatctgcttgatactattgcagtggcagttggcaattggactgacagcatcatcttaggtatacaaagaatagggcgaggattcctcaacatcccaagaagaattagacaaggtgcagaaagagctctaaattaa

SEQ ID NO: 35 - protein, 884 aa, HIV-1
  VARIANT (0)...(0): Xaa = any amino acid
MGGMRAMKKKKNSLGNLEICLALVIYFNAISCASGIHYVTVYYGVPVWRNAEVTLFCAADASLTSKEQHNIWATQACVPTDPTPIEVKINVTESFNIWKNYMVTQMQEDIISLWDQSLKPCVKLTILCVTMNCSECHKQAKCNNPSENRTAANPLELFKCSFNTTTVLKDKKQEQQALFYRTDLIALNETANNTLYRLINCNTTTITQACPKVTFEPLPIQYCAPAGYALMKCXQXGFNGTGPCNQTVITHCTHGIKPTVSTQLILNGTLAEXEPLVITQNVSDTRYVIIVKLNKNVSLTCVRPGNNTRXQVQIGPMTWYNMKFYTGDIRKAYCNXSMQXWTKTLESVSKAIWKAYPQPPNQNHTFVFRNSTGGDPEVSFLHFSCHGEFFYCNTSSLFNYSYTCNEKGVCSINNHTGNYTGENIITLPCRLKQVVNSWMRVGSGLFAPPIEGQLQCHSNITGLILDRASPYNANSSSNTTLSPTGGDMRHIWRSELYPYKVVQVKALAVAPTRVSRPTIMXHDAHRKKRGAGLGMLFLGFMSAAGSTMGAAAVTLTVQARQVLHGIVQQQNNMLRAIEAQQELLRLSVWGIRQLRARLLAIETYLRDQQLLGLWGCSGQIVCYTNVPWNRSWTNKSETELDGXWTNLTWQEWDKLVDNYTDTIYLEIQRAQDQQKANEKKLLELDQWAQLWNWLDITQWLWYIKIFIMIVGGIIGLRILLAXXNVVRRIRQGYSPVSLQTLGLNGDPAGIAPGTNEEGGEAGNGRSIRLLDGFLPLVWDDLKNLVVQIYQILVGCILGIKDLLTILWIHLGQLLTRGLNCLRDCFAACGYWTQELKQSATSLLDTVAISVAGWTDQVIIVGQQIGRGFLNIPRRIRQGIERSLL

SEQ ID NO: 36 - protein, 847 aa, HIV-1
MRVKEIQRNYQHLWKWSLIILGMIMICKAIEKSWVTVYYGVPVWKDAETTLFCASDAKAYEKESHNVWATHACVPTDPSPQELVLGNVTENFNMWKNKMVEQMHEDIISLWDQSLKPCVKLTFLCVTLNCIDVKNSTNNNTEEATITNCSFKVPTELKDKTETVHTLFYKLDVVPLNVTNNSSISSTYRLINCNTSTITQACPKVSFEPIPIHYCAPAGFAILKCNDKKFNGTGPCKNVSTVQCTHGIRPVVSTQLLLNGSLSEEEVIIRSENITNNAKTIIVQLNETVKINCTRPGSDKKIRQSIRIGPGKVFYAKGGITGQAHCNITDGEWRNTLQQVAIALRRQFNNKSIIFNSSSGGDIEITTHTFNCGGEFFYCNTSELFTGIWNGTWDKNCTSTESNCTGNITLPCRIKQVVRTWQGVGQAMYAPPIEGTIRCSSNITGLLLTRDGGNGNATQNETFRPGGGDMKDNWRSELYKYKVVKIEPLGVAPTRAKRRVVEREKRAVGMGALFLGFLGAAGSTMGAASMALTAQARQLLSGIVQQQNNLLRAIEAQQHLLQLTVWGIKQLQARVLAVERYLESQQLLGLWGCSGKLICTTTVPWNSSWSNKSLDNIWDNLTWMEWDREISNYTQVIYGLLEDSQKQQEKSEKDLLELDKWASLWNWFDITNWLWYIKIFIMIVGGLIGLRIVFTVFSIINRVRQGYSPLSFQTLLPTPRGPDRPGRTEEEGGEEDNNRSVRLVNGFLALAWEDLRSLCIFSYHRLRDLILIVVKGLRRGWEALKYLGNLVLYWGQELKNSAISLLNATAIVVAEGTDRIIEVGQRICRAILNIPRRIRQGFERALL

SEQ ID NO: 37 - DNA, 2544 bp, HIV-1
atgagagtgaaggagatacagaggaattatcaacacttgtggaaatggagcctgataattttaggaatgataatgatatgtaaagctatagaaaaatcgtgggtcacagtctattatggggtacctgtgtggaaagatgcagaaaccactctattttgtgcatcagatgctaaggcatatgagaaagaatcgcataatgtctgggctacacatgcctgtgtacccacagaccccagcccacaagagctagttttgggaaatgtaacagaaaactttaacatgtggaagaataaaatggtagagcagatgcatgaggatataatcagtttatgggatcaaagccttaagccatgtgtaaagttaacctttctttgtgtcactttaaactgtattgatgtaaagaatagtactaacaataacactgaagaagctaccatcacaaattgctccttcaaggtacccacagaactgaaagataagacggagacagtacatacacttttttataaactggatgtagtgccacttaatgtgacaaataattctagtataagtagtacctataggttaataaattgtaatacctcaaccattacacaggcttgtccaaaggtatcctttgagccaattcctatacattattgtgcccctgctggttttgcgattctaaagtgtaatgataagaagttcaatggaacaggaccatgcaaaaatgtcagcacagtacaatgcacacatggaattaggccagtggtgtcaactcaattactattaaatggcagtttatcagaagaagaggtaataattagatctgaaaatatcacaaacaatgccaaaaccataatagtacagcttaatgagactgtaaaaattaattgtaccagacccggatccgacaagaagataagacaaagtatacgtataggaccaggaaaagtattctatgcaaaaggtggaataacaggacaagcacattgtaacattacagatggggaatggaggaatactttacaacaggtagctatcgcattaagaagacaatttaataataaatcaataatatttaactcatcctcaggaggggacatagagattacaacacatacttttaactgtggaggagagtttttctattgcaacacatcagagctgtttactggtatttggaatggtacttgggataagaattgcactagcactgagagtaattgcactggaaatattacactcccatgcaggataaaacaagtggtaagaacatggcagggagtaggacaagcaatgtatgcccctcctatcgaagggacaattaggtgctcatcaaatattacaggtctactattgacaagagatggtggtaatggcaatgcaactcaaaatgagacctttagacctggaggaggagacatgaaagataattggagaagtgaattgtataagtataaagtagtaaaaattgaaccactaggagtagcacccaccagggcaaaaagaagagtggtggagagagaaaaaagagcagtggggatgggagctttgtttctcgggttcttgggagcagccggaagcactatgggcgcggcgtcaatggcgctgacggcacaggccagacaattattgtctggtatagtgcagcagcaaaacaatttgctgagggctatagaggcgcaacagcatctgttgcaactcacagtctggggcattaaacagctccaggcaagagtcctggctgtggaaagatacctagagagtcaacagctcctagggctttggggctgctctggaaaactcatctgcaccactactgtgccctggaactctagctggagtaataaatccttggataacatttgggacaatctgacctggatggagtgggatagagaaattagcaattacacacaagtaatatatgggttgcttgaagactcacaaaaacagcaggaaaagagtgaaaaagatttactggaattggataagtgggcaagtctgtggaactggtttgacataacaaattggttgtggtatataaaaatattcataatgatagtaggaggcttgataggcttaagaatagtttttactgtgttttctataataaatagagttaggcagggatactcacctttgtctttccagaccctcctcccaaccccgaggggacccgacaggccaggaagaaccgaagaagaaggtggagaagaagacaacaacagatccgttcgattagtgaacggattcttagcacttgcctgggaagacctgcggagcctgtgcatcttcagctaccaccgcttgagagacttaatcttgattgtagtaaagggactgcgacgggggtgggaagcactcaaatacctggggaatcttgtgctgtattggggtcaggaactaaagaatagtgctattagtttgcttaatgccacagcaatagtagtagctgagggaacagatagaattatagaagtgggacaaagaatttgtagggctattctcaatatacctagaagaataagacagggtttcgaaagggctttactgtaa

SEQ ID NO: 38 - protein, 859 aa, HIV-1
MRVMGIQKNYPLLWRWGMIIFWIMTICSAGNLWVTVYYGVPVWRDAETTLFCASDAKAYDTEVHNVWATHACVPTDPNPQEIHLGNVTEDFNMWKNSMVEQMHEDIISLWDQSLKPCVQLTPLCVTLHCQDNLTSSGNISENMQGEIKNCSFNMTTELRDKKQKVYALFYRYDVVQINETGDNIQYRLINCNTSAITQACPKVSFEPIPIHYCAPAGFAILKCNDEKFNGTGPCKNVSTVQCTHGIKPVVSTQLLLNGSLAEEEIVIRSENFTNNAKIIIVQLHESVKINCTRPGNNTRKSVRIGPGQTFYATGDIIGDIRQAHCNVSWQQWNKTLHDVATKLREYFNNTTIIFDEPSGGDLEITTHSFNCGGEFFYCNTSNLFNRTWNHNGTWNAPGPFNDTEDKTINGTEDKTITLQCRIKQIVRMWQKVGQAMYAPPIPGEIRCESNITGLLLTRDGGNDNNNTETFRPGGGDMRDNWRSELYKYKVVKIEPLGVAPSHAKRRVVEREKRALVGLGAFFFGFLGAAGSTMGAASITLTVQARQLLSGIVQQQSNLLKAIEAQQHLLRLTVWGIKQLQARVLALEAYLKDQQLLGIWGCSGKLICTTTVPWNSSWSNKTYDHIWGNMTWLQWDKEISNYTHIIYDLIEESQNQQEKNEQDLLALDKWASLWDWFSISSWLWYIRIFIIIVGGLIGLRIVFAVLAIINRVRQGYSPLSFQTLTHHQREPGRPERIEEGGGGQDRDRSVRLVSGFLALAWDDLRSLCLFSYHRLRDFVSIVARTVELLGHRGWEALKYLWNLLSYWGQELKNSAISLLDTIAIVVANWTDRVIELVQRAGRAILNIPRRIRQGFERALL

SEQ ID NO: 39 - DNA, 2580 bp, HIV-1
atgagagtgatggggatacagaagaattatccactcttatggagatggggtatgataatattttggataatgacaatttgtagtgctggaaatttgtgggtcacggtctattatggggtacctgtgtggagagacgcagagaccaccctattttgtgcatcagatgctaaagcatatgatacagaagtacataatgtctgggctacacatgcctgcgtacccacagaccctaacccacaagaaatacatttgggaaatgtaacagaagattttaacatgtggaaaaatagcatggtagagcagatgcatgaagatataattagtctatgggatcaaagcctaaagccatgtgtacagttaacccctctctgtgttactttacattgtcaggataacctcactagcagcggcaacatatcggaaaacatgcaaggagaaataaaaaactgctctttcaatatgaccacagaactaagagataagaaacagaaagtgtatgcacttttttatagatatgatgtagtacaaattaatgaaactggggataacattcaatataggttaataaattgtaatacctcagccattacacaggcttgtccaaaggtatcctttgagccaattcccatacattattgtgccccagctggctttgcaattctaaagtgtaatgatgagaagttcaatggaacagggccatgcaagaatgtcagcacagtacaatgcacacatggaatcaagccagtagtatcaactcaactgttattaaatggcagcctagcagaagaagagatagtgattagatctgaaaattttacaaacaatgccaaaatcataatagtacagttgcatgaatctgtaaaaattaattgtaccagacctggcaacaatacaagaaaaagtgtacgtataggaccagggcaaacattctatgcaacaggtgacataataggggatataagacaagcacattgtaatgtcagctggcaacaatggaacaaaactttacacgatgtggctacaaaattaagggagtattttaataataccacaataatctttgatgaaccctcaggaggggatttagaaattacaacacatagttttaattgtggaggagaatttttctattgcaatacatcaaatctgtttaatagaacttggaatcataatggcacttggaatgcaccaggaccgtttaatgacactgaggataaaacaataaatggcactgaggataaaacaataactctccaatgcagaataaagcaaattgtgcgtatgtggcagaaagtaggacaagcaatgtatgcccctcccatcccaggagaaataaggtgtgaatcaaacattacaggactactattaacaagagatggagggaatgataataataatacagagaccttcaggcctggaggaggagatatgagggacaattggagaagtgaattatataaatataaagtagtaaaaattgaaccactaggtgtagcaccctcccatgcaaaaagaagagtggtggagagagaaaaaagagcacttgttggactgggagctttcttctttgggttcttaggagcagcaggaagcactatgggcgcggcgtcaataacgctgacggtacaggccagacaattattgtctggtatagtgcaacagcagagcaatctgctgaaggctatagaggctcaacaacatctgttgagactcacggtctggggcattaaacagctccaggcaagagtcctggctctagaagcatacctaaaggatcaacagctcctaggaatttggggctgctctggaaaactcatctgcaccactactgtaccctggaactctagttggagtaataaaacttatgatcacatatggggtaacatgacctggctgcaatgggataaagaaattagtaactacacacacataatatatgatctaattgaagaatcgcagaaccagcaggaaaagaatgaacaagacttattggcattggacaagtgggcaagtctgtgggattggtttagcatatcaagttggctatggtatataagaatatttataataatagtaggaggtttaataggcttaagaatagtctttgctgtacttgctataataaatagagttaggcagggatactcacctttgtctttccagacccttacccaccaccagagggaacccggcaggcccgaaagaatcgaagaaggaggtggcgggcaagacagagacagatccgtgcgattagtgagcggattcttagcacttgcctgggacgatctgcggagcctgtgcctcttcagctaccaccgattgagagacttcgtctcgattgtagcgaggactgtggaacttctgggacacagggggtgggaagccctcaaatatctgtggaatcttctatcgtactggggtcaggaactaaagaatagtgctattagtttgcttgataccatagcaatagtagtagctaattggacagacagagttatagaactagtacaaagagctggtagagctattctcaacatacctaggagaatcagacagggctttgaaagggctttgctataa

SEQ ID NO: 40 - protein, 859 aa, HIV-2
MCGRNQLFVASLLASACLIYCVQYVTVFYGVPVWRNASIPLFCATKNRDTWGTIQCLPDNDDYQEIALNVTEAFDAWNNTVTEQAVEDVWSLFETSIKPCVKLTPLCVAMRCNSTTAKNTTSTPTTTTTANTTIGENSSCIRTDNCTGLGEEEMVDCQFNMTGLERDKKKLYNETWYSKDVVCESNDTKKEKTCYMNHCNTSVITESCDKHYWDTMRFRYCAPPGFALLRCNDTNYSGFEPNCSKVVAATCTRMMETQTSTWFGFNGTRAENRTYIYWHGRDNRTIISLNKFYNLTVHCKRPGNKTVVPITLMSGLVFHSQPINRRPRQAWCWFKGEWKEAMKEVKLTLAKHPRYKGTNDTEKIRFIAPGERSDPEVAYMWTNCRGEFLYCNMTWFLNWVENRTNQTQHNYVPCHIKQIINTWHKVGKNVYLPPREGQLTCNSTVTSIIANIDGGENQTNITFSAEVAELYRLELGDYKLIEVTPIGFAPTPVKRYSSAPVRNKRGVFVLGFLGFLTTAGAAMGAASLTLSAQSRTLLAGIVQQQQQLLDVVKRQQEMLRLTVWGTKNLQARVTAIEKYLKDQAQLNSWGCAFRQVCHTTVPWVNDTLTPDWNNMTWQEWEQRIRNLEANISESLEQAQIQQEKNMYELQKLNSWDVFGNWFDLTSWIKYIQYGVYIVVGIIVLRIVIYVVQMLSRLRKGYRPVFSSPPAYFQQIHIHKDREQPAREETEEDVGNSVGDNWWPWPIRYIHFLIRQLIRLLNRLYNICRDLLSRSFQTLQLISQSLRRALTAVRDWLRFNTAYLQYGGEWIQEAFRAFARATGETLTNAWRGFWGTLGQIGRGILAVPRRIRQGAEIALL

SEQ ID NO: 41 - DNA, 2580 bp, HIV-2
atgtgtggtaggaatcaactatttgttgccagcttgctagctagtgcttgcttaatatattgcgtccaatatgtgactgttttctatggcgtgcccgtgtggagaaatgcatccattcccctcttttgtgcaactaaaaatagagatacttggggaaccatacagtgcttgccagacaatgatgactatcaggaaatagctttaaatgtgacagaggccttcgacgcatggaataatacagtaacagaacaagcagtagaagatgtctggagtctatttgagacatcaataaaaccatgcgtcaaactaacacccttatgtgtagcaatgcgttgtaacagcacaactgcaaaaaacacaacctccacaccaacaaccaccacaacagcaaacacaacaataggagagaattcttcatgcatacgcacagacaactgcacagggttgggagaagaagagatggtcgactgtcagttcaatatgacaggattagagagggataagaaaaaactatataatgaaacatggtactcaaaagatgtagtctgtgaatcaaatgacaccaagaaagagaaaacatgttacatgaaccactgcaacacatcagtcatcacagagtcatgtgacaagcactattgggatactatgaggtttagatattgtgcaccaccgggttttgccctgctaagatgcaatgataccaattattcaggctttgagcccaattgttctaaggtagtagctgctacatgtacaaggatgatggaaacgcaaacctccacttggtttggctttaatggcactagggcagaaaatagaacatatatctattggcatggtagggataatagaactatcattagcttaaacaagttttataatctcaccgtacattgtaagaggccaggaaacaagacagttgtaccaataacactcatgtcagggttagtgtttcactcccagccaatcaatagaagacccaggcaagcatggtgctggttcaaaggcgagtggaaggaagccatgaaggaggtgaagctaacccttgcaaaacatcccaggtataaaggaaccaacgacacagaaaaaattcgttttatagcgccaggagaacgctcagacccagaagtggcatacatgtggactaactgcagaggagaatttctctactgcaatatgacttggttcctcaattgggtagaaaacagaacgaatcagacacagcacaattatgtgccatgccatataaagcaaataattaatacctggcacaaggtagggaaaaatgtatatttgcctcctagggaaggacagttaacctgcaactctacagtgaccagcataattgctaacattgacggaggagagaaccagacaaatattacctttagtgcagaggtggcagaactataccgattagaattgggggattataaattgatagaagtaacaccaattggctttgcacctacaccagtaaaaagatactcctctgctccagtgaggaataaaagaggtgtattcgtgctagggttcttaggttttctcacgacagcaggagctgcaatgggcgcggcgtccttgacgctgtcggctcagtctcggactttattggccgggatagtgcagcaacagcaacagctgttggacgtggtcaagagacaacaagaaatgttgcgactgaccgtctggggaacaaaaaatctccaggcaagagtcactgctatcgagaaatacttaaaggaccaggcgcaactaaattcatggggatgtgcgtttagacaagtctgccacactactgtaccatgggtaaatgacaccttaacgcctgattggaacaacatgacatggcaggaatgggagcaacgaatccgcaacctagaggcaaatatcagtgaaagtttagaacaggcacaaatccagcaagaaaagaacatgtatgaactacaaaaattaaatagctgggatgtttttggcaactggtttgatttaacctcctggatcaaatatattcagtatggagtttatatagtagtaggaataatagttttaagaatagtaatatatgtagtacaaatgttaagtagacttagaaagggctataggcctgttttctcttccccccccgcttacttccaacagatccatatccacaaggaccgggaacagccagccagagaagaaacagaagaagacgttggaaacagcgttggagacaattggtggccctggccgataagatatatacatttcctgatccgccagctgattcgcctcttgaacagactatacaacatctgcagggacttactatccaggagcttccagaccctccaactaatctcccagagtcttcggagagcattgacagcagtcagagactggctgagatttaacacagcctacctgcaatatgggggcgagtggatccaagaagcgttccgagccttcgcgagggctacgggagagactcttacaaacgcctggagaggcttctgggggacactgggacaaattgggaggggaatacttgcagtcccaagaaggatcaggcagggggcagaaatcgccctcctgtga

SEQ ID NO: 42 - protein, 727 aa, SIV
ESFDAWENTVTEQAIEDVWQLFETSIKPCVKLSPLCITMRCNKSETDKWGLTKSSTTTTATTATPASTTRTTSAKIDMVNETSSCITHNNCTGLEQEQMISCKFNMTGLKRDKKKEYNETWYSTDLVCEQGNSTDNESRCYMNHCNTSVIQESCDKHYWDTIRFRYCAPPGYALLRCNDTNYSGFMPKCSKVVVSSCTRMMETQTSTWFGFNGTRAENRTYIYWHGKDNRTIISLNKYYNLTMKCRRPGNKTVLPVTIMSGLVFHSQPINDRPKQAWCWFGGNWKDAIKEVKQTIVKHPRYTGTNNTDKINLTAPRGGDPEVTFMWTNCRGEFLYCKMNWFLNWVEDRNLTNKKSKEQHKRNYVPCHIRQIINTWHKVGKNVYLPPREGDLTCNSTVTSLIANIDWTDGNQTNITMSAEVAELYRLELGDYKLVEITPIGLAPTDVKRYTTGGTSRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPDWNNDTWQEWERKVDFLEENITALLEEAQIQQEKNMYELQKLNSWDVFGNWFDLASWIRYIQYGIYIVVGVILLRIVIYIVQILAKLRQGYRPVFSSPPSYSQQTHIQQDPALPTREGKEGDGGESGGNSSWPWQIEYIHFLIRQLIRLLTWLFNNCRTLLSR

SEQ ID NO: 43 - DNA, 2182 bp, SIV
gaaagctttgatgcttgggagaatacagtcacagaacaggcaatagaggatgtatggcaactctttgagacctcaataaagccttgtgtaaaattatccccattatgcattactatgagatgcaataaaagtgagacagataaatggggattaacaaaatcatcaacaacaacaacagcaacaacagcaacaccagcatcaacaacaaggacaacatcagcaaaaatagacatggtcaatgagactagttcttgtataactcataataattgcacaggcttggaacaagagcaaatgataagctgtaagttcaacatgacagggttaaaaagagacaagaaaaaggagtacaatgaaacttggtactctacagatttggtttgtgaacaagggaatagcactgataatgaaagtagatgctacatgaatcactgtaacacttctgttatccaagagtcttgtgacaagcattattgggatactattagatttaggtattgtgcacctccaggttatgctttgcttagatgtaatgacacaaattattcaggctttatgcctaaatgttctaaggtggtggtctcttcatgcacaaggatgatggagacacagacttctacttggtttggctttaatggaactagagcagaaaatagaacttatatttactggcatggtaaagataataggactataattagtttaaataagtattataatctaacaatgaaatgtagaagaccaggaaataagacagttttaccagtcaccattatgtctggattggttttccactcacaaccaatcaatgataggccaaagcaggcatggtgttggtttggaggaaattggaaggatgcaataaaagaggtgaagcagaccattgtcaaacatcccaggtatactggaactaacaatactgataagatcaatttgacggctcctagaggaggagatccggaagttaccttcatgtggacaaattgtagaggagagtttctctactgtaaaatgaattggtttctaaattgggtagaagataggaatctaactaacaagaagtcaaaggaacagcataaaaggaattacgtgccatgtcatattagacaaataatcaacacttggcataaagtaggcaaaaatgtttatttgcctccaagagagggagacctcacgtgtaactccacagtgaccagtctcatagcaaacatagattggactgatggaaaccaaactaatatcaccatgagtgcagaggtggcagaactgtatcgattggaattgggagattataaattagtagagatcactccaattggcttggcccccacagatgtgaagaggtacactactggtggcacctcaagaaataaaagaggggtctttgtgctagggttcttgggttttctcgcaacggcaggttctgcaatgggcgcggcgtcgttgacactgaccgctcagtcccggactttattggctgggatagtgcagcaacagcaacagctgttggacgtggtcaagagacaacaagaattgttgcgactgaccgtctggggaacaaagaacctccagactagggtcactgccatcgagaagtacttaaaggaccaggcgcagctgaatgcttggggatgtgcgtttagacaagtctgccacactactgtaccatggccaaatgcaagtctaacaccagactggaacaatgatacttggcaagagtgggagcgaaaggttgacttcttggaggaaaatataacggcccttctagaagaggcacaaattcaacaagagaagaacatgtatgaattacaaaagttgaatagctgggatgtgtttggcaattggtttgaccttgcttcttggataaggtatatacaatatggaatttatatagttgtaggagtaatactgttaagaatagtgatctatatagtacaaatactagctaagttaaggcaggggtataggccagtgttctcttccccaccctcttattcccagcagacccatatccaacaggacccggcactgccaaccagagaaggcaaagaaggagacggtggagaaagcggtggcaacagctcctggccttggcagatagaatatattcatttcctgatccgccaactgatacgcctcttgacttggctattcaacaactgcagaaccttgctatcgagag

SEQ ID NO: 44 - protein, 858 aa, SIV
  VARIANT (0)...(0): Xaa = any amino acid
MRKPIHIIWGLALLIQFIEKGTNEDYVTVFYGVPVWRNATPTLFCATNASMTSTEVHNVWATTSCVPIDPDPIVVRLNTSVWFNAYKNYMVESMTEDMXQLFQQSHKPCVKLTPMCIKMNCTGYNGTPTTPSTTTSTVTPKTTTPIVDGMKLQECNFNQSTGFKDKKQKMKAIFYKGDLMKCQDNNETNCYYLWHCNTTTITQSCEKSTFEPIPIHYCAPAGYAILRCEDEDFTGVGMCKNVSVVHCTHGISPMVATWLLLNGTYQTNTSVVMNGRKNESVLVRFGKEFENLTITCIRPGNRTVRNLQIGPGMTFYNVEIATGDTRKAFCTVNKTLWEQARNKTEHVLAEHWKKVDNKTNAKTIWTFQDGDPEVKVHWFNCQGEFFYCDITPWFNATYTGNLITNGALIAHCRIKQIVNHWGIVSKGIYLAPRRGNVSCTSSITGIMLEGQIYNETVKVSPAARVADQWRAELSRYQVVEIXPLSVAPTTGKRPEIKQHSRQKRGIGIGLFFLGLLSAAGSTMGAASIALTAQTRNLXHGIVQQQANLLQAIETQQHLLQLSVWGVKQLQARMLAVEKYLRDQQLLSLWGCADKVTCHTTVPWNNSWVNFTQTCAKNSSDIQCIWENMTWQEWDRLVQNSTGQIYNILQIAHEQQERNKKELYELDKWSSLWNWFDITQWLWYIKIFIMIVGAIVGLRILLVLVSCLRKVRQGYHPLSFQIPTQNQQDPEQPEEIREEGGRKDRIRWRALQHGFFALLWVDLTSIIQWIYQICRTCLLNLWAVLQHLCRITFRLCNHLENNLSTLWTIIRTEIIKNIDRLAIWVGEKTDSILLALQTIVRIIREVPRRIRQGLEIALN

SEQ ID NO: 45 - protein, 882 aa, SIV
MGCLGNQLLIAILFLSAYGIYCIQYVTVFYGVPAWRNATIPLFCVTRNRDTWGTTQCLPDNDDYSELALNITESFDAWENTVTEQAIEDVWHLFETSIKPCVKLTPLCITMKCNKSETDKWGLTKSSTTTAAPTTKTTTTKEIEVVNENSTCVNRDNCTGLEQEPMISCKFNMTGLKRDKKREYNETWYSADLVCEQGNSTEDESRCYMNHCNTSVIQESCDKHYWDAIRFRYCAPPGYALLRCNDTKYSGFMPNCSKVVVSSCTRMMETQTSTWFGFNGTRAENRTYIYWHSKDNRTIISLNKYNNLTMKCRRPGNKTVLPVTIMSGLVFHSQPINERPKQAWCRFEGNWKEAIKEVKQTIVKHPRYTGTNNTDKINLTAPRGGDPEVTFMWTNCRGEFLYCKMNWFLNWVEDKNLTGTTQKPQEQHKRNYVPCHIRQIINTWHKVGKNVYLPPREGDLTCNSTVTSLIANIDWIDGNQTNITMSAEVAELYRLELGDYKLVEITPIGLAPTNVKRYTTGGTPRNKRGVFVLGFLGFLATAGSAMGAASLTLTAQSRTLLAGIVQQQQQLLDVVKRQQELLRLTVWGTKNLQTRVTAIEKYLKDQAQLNAWGCAFRQVCHTTVPWPNASLTPNWNNETWQEWERKVDFLEENITALLEEAQIQQEKNMYELQKLNSWDVFGNWFDLASWIRYIQYGVYIVVGVILLRIVIYIVQMLAKLRQGYRPVFSSPPSYFQQTHIRQDQALPTKEGTEGDGGDSGGNSSWPWQIEYIHFLIRQLIRLLTWLFSNCRTLLSRAYQILQPIFQRFSTTLQRVREVLRTELTYLQYGWSYFQEAVQVAWRSATETLAGAWGDLWETLGRVGRWILAIPRRIRQELELTLL

SEQ ID NO: 46 - DNA, 2649 bp, SIV
atgggatgtcttgggaatcagctgcttatcgccatcttgtttctaagtgcctatgggatctattgcattcaatatgtcacagtcttttatggtgtaccagcttggaggaatgcgacaattcccctcttctgtgtaaccaggaatagggatacttggggaacaactcagtgcctaccagataatgatgattattcagaattggcccttaatattacagaaagctttgatgcttgggagaatacagtcacagaacaggcaatagaggatgtatggcatctctttgagacctcaataaagccttgtgtaaaattaaccccattatgcattactatgaaatgcaacaaaagtgaaacagataaatggggattgacaaaatcatcaacaacaacagcagcaccaacaacaaaaacaacaacaacaaaggaaatagaagtggtcaatgaaaatagtacttgtgtaaatcgtgataattgcacaggcttggaacaagagccaatgataagctgtaaattcaacatgacagggttaaaaagagacaagaaaagagagtacaatgaaacttggtactctgcagatttggtttgtgaacaaggtaatagcactgaagatgaaagtagatgttacatgaatcactgtaacacttctgttattcaagaatcttgtgacaaacattattgggatgctattagatttaggtattgtgcacctccaggttatgctttgcttagatgtaatgacacaaagtattcaggctttatgcctaactgttctaaggtggtggtctcttcatgcacaagaatgatggagacacagacttctacttggtttggctttaatggaactagagcagaaaatagaacttatatttactggcatagcaaagataataggactataattagtttgaataagtataataatctaacaatgaaatgtagaagaccaggaaataagacagttttaccagtcaccattatgtctggattggttttccactcacaaccaatcaatgaaaggccaaaacaggcatggtgtaggtttgaaggaaattggaaggaggcaataaaagaggtgaagcagaccattgtcaaacatcccaggtatactggaactaacaatactgataaaatcaatttgacggctcctcgaggaggagatccggaagttaccttcatgtggacaaattgcagaggagagtttctctactgtaaaatgaattggtttctaaattgggtagaagataagaatctgactggaactacccagaagccacaggaacagcataaaaggaattacgtgccatgtcatattagacaaataatcaacacttggcataaagtaggcaaaaatgtttatttgcctccaagagagggagacctcacgtgtaactccacagtaaccagtctcatagcaaacatagattggattgatggaaaccaaactaatatcaccatgagtgcagaggtggcagaactgtatcgattggaattgggagattataaattagtagagatcactccaattggcttggcccccacaaatgtgaagaggtacactactggtggcaccccaagaaataaaagaggggtctttgtgctagggttcttaggttttctcgcaacggcaggttctgcaatgggcgcggcgtcgttgacgctgaccgctcagtcccggactttattggctgggatagtgcagcaacagcaacagctgttggacgtggtcaagagacaacaagaattgttgcgactgaccgtctggggaacaaagaacctccagactagagtcactgccatcgagaagtacttaaaggaccaggcgcagctaaatgcttggggatgtgcatttagacaagtctgccatactactgtaccatggccaaatgcaagtctaacaccaaattggaacaatgagacttggcaagagtgggagcgaaaggttgacttcttggaggaaaatataacggcccttctagaagaggcacaaattcaacaagaaaagaacatgtatgaattacaaaagttgaatagctgggatgtgtttggcaattggtttgaccttgcttcttggataaggtatatacaatacggagtttatatagttgtaggagtaatactgttaagaatagtcatctatatagtacaaatgctagctaagttaaggcaagggtataggccagtgttctcttccccaccttcttatttccagcagacccatatccgacaggaccaagcactgccaaccaaagaaggaacagaaggagacggtggagacagcggtggcaacagttcctggccttggcagatagagtatattcatttcctgatccgccaactgatacgcctcttgacttggctattcagcaactgcagaaccttgctatcgagagcataccagatcctccaaccaatattccagagattctccacgaccctacagagagtccgagaagtcctcaggactgaactaacctacctacaatatgggtggagctacttccaagaagcggtccaagtcgcctggagatctgcgacagagactcttgcgggcgcgtggggagacttatgggagactctgggaagggttggaagatggatactcgcaatccctaggaggatcagacaagagcttgagcttactctcttgtga

(19) United States
(12) Patent Application Publication
(10) Pub. No.: US 2013/0101617 A1
(43) Pub. Date: Apr. 25, 2013

(54) ENV TRIMER IMMUNOGENS

(71) Applicant: James Binley, San Diego, CA (US)

(72) Inventor: James Binley, San Diego, CA (US)

(73) Assignee: TORREY PINES INSTITUTE FOR MOLECULAR STUDIES, San Diego, CA (US)

(21) Appl. No.: 13/805,637

(22) PCT Filed: Jun. 29, 2011
(86) PCT No.: PCT/US11/42441

§ 371 Date: Dec. 19, 2012

Related U.S. Application Data
(60) Provisional application No. 61/360,067, filed on Jun. 30, 2010.

(51) Int. Cl. A61K 39/385

(52) U.S. Cl. CPC A61K 39/385
USPC 424/196.11; 435/68.1; 435/7.1

(57) ABSTRACT

Embodiments of the present invention are drawn to pure forms of human or simian immunodeficiency virus trimeric gp120/gp41 Env protein (Env trimers) and methods for making them. These embodiments address the need for an authentic immunogen lacking uncleaved gp160 Env protein and/or other forms of Env, such as gp41 “stumps” dissociated from gp120, which interfere with neutralizing antibody production in a vaccinated subject.

### CROSS-REFERENCE TO RELATED APPLICATIONS

This application claims priority under 35 U.S.C. §119(e) to U.S. Provisional Patent Application Ser. No. 61/360,067, filed on Jun. 30, 2010, which is incorporated herein by reference in its entirety, and claims priority under 35 U.S.C. 371(c) to International Application No. PCT/US11/42441, filed Jun. 29, 2011, which is incorporated herein by reference in its entirety.

### REFERENCE TO SEQUENCE LISTING

The present application is being filed along with a Sequence Listing in electronic format. The Sequence Listing is provided as a file entitled TPIMS006NP_SEQUENCE_LISTING.TXT, created Dec. 12, 2012, which is 240 KB in size. The information in the electronic format of the Sequence Listing is incorporated herein by reference in its entirety.

### FIELD OF THE INVENTION

Embodiments of the present invention are drawn to pure forms of human or simian immunodeficiency virus trimeric gp120/gp41 Env protein (Env trimers) and methods for making them. These embodiments provide an authentic immunogen lacking uncleaved gp160 Env protein and/or other forms of Env, such as gp41 “stumps” dissociated from gp120, that interfere with neutralizing antibody production in a vaccinated subject.

### BACKGROUND

Broadly neutralizing antibodies (nAbs) may be a crucial component of vaccine-elicited protective immunity against HIV-1. However, all attempts to elicit such responses to date have failed. The lack of progress in this area could relate to the insufficient authenticity of candidate immunogens. Indeed, a consistent finding of immunogenicity studies has been that while immune sera efficiently bind to the immunogen, little or no Abs recognize the authentic form of Envelope glycoprotein (Env) found on virus particles. Since nAbs have the select ability to bind to these Env spikes, authentic gp120/gp41 trimers may in turn be the only antigen capable of selectively inducing nAbs in a vaccine setting. Testing this possibility has until now been problematic. An enduring challenge has been one of purity. As an example, particulate vaccines typically bear authentic gp120/gp41 trimers and various forms of non-functional Env on their surfaces. The latter are “promiscuous”, in that they are recognized by non-neutralizing Abs. Their presence on particles may interfere with the development of neutralizing responses.

Broadly neutralizing antibodies (nAbs) are likely to be a crucial component of vaccine-elicited protective immunity against HIV-1. However, all attempts to elicit such responses have to date been disappointing. A common problem is that although candidate immunogens elicit effective responses against themselves, immune sera generally recognize the native trimeric gp120/gp41 Envelope glycoprotein (Env) very poorly. This implies a lack of sufficient immunogen authenticity. Another problem is that non-functional forms of Env exist on HIV-1 surfaces. As a further problem, responses to each available Env target are not generated equally, but rather apparently in a hierarchical manner. For example, in the case of natural HIV-1 infection, Ab responses to non-functional Env appear to take precedence over those against authentic gp120/gp41 trimers, the latter typically taking several months to develop. Vaccine research has been hampered by similar problems: despite the presence of native Env gp120/gp41 trimers in particle immunogens, alternative forms of Env tend to be antigenically “promiscuous” and may interfere with the emergence of anti-gp120/gp41 trimer responses.

One approach towards testing the possibility of using native trimers as immunogens is to use virus-like particle (VLP) vaccines (16, 20, 35, 36, 49, 77, 100). However, virus-like particle (VLP) vaccines have to date not achieved much success for HIV-1. This is perhaps because Env is not biochemically homogeneous—in addition to native gp120/gp41 trimers, particles bear non-functional Env, such as uncleaved gp160 or gp41 stumps (51, 65, 76). These antigenically “promiscuous” antigens appear to draw B cell responses at the expense of those against native gp120/gp41 Env trimers, resulting in overwhelmingly non-neutralizing responses (20). The immunogenic pre-eminence of aberrant Env is demonstrated by the efficient capture of virus by many non-neutralizing mAbs (70) and also by the rapid emergence of virus-Ig immune complexes during natural infection, in the absence of any neutralization (94). Even in natural infection, bnAbs are generated in only ˜10% of patients and require significant time and sustained virus replication to develop (25, 83). The difficulty in retrieving monoclonal Abs (mAbs) that mirror these responses is also a testament to their scarcity (13, 18, 56, 87, 101).

A recent clinical trial suggests that an HIV-1 vaccine may be possible (80). Optimal efficacs ray require a component that induces broadly neutralizing antibodies (bnAbs). These block virus infection by binding to functional envelope glycoprotein (Env) spikes on particle surfaces (19, 31, 37, 41, 74, 75, 85). These spikes consist of non-covalently associated trimers of gp120 surface subunits and gp41 transmembrane-anchoring subunits. This complex has evolved to be compact and highly glycosylated, ostensibly to disfavor nAb binding. Quaternary associations occlude multiple determinants that are exposed on other forms of Env, such as soluble gp120.

All Env-based vaccine candidates have so far failed to induce bnAbs. Instead, they largely elicit Abs against determinants that are not exposed on authentic spikes (48), suggesting a lack of stringency in the Ab specificities they induce. This point is illustrated by new structural information that indicates almost identical gp120 binding mechanisms for neutralizing and non-neutralizing mAbs that target the CD4 binding site (15). Subtle differences in specificity appear to render the virus resistant to all but the most accurately targeted Abs. This being the case, insufficiently authentic immunogens cannot selectively elicit the exquisite specificities necessary to achieve neutralization.

### SUMMARY OF THE INVENTION

The embodiments of the present invention provide a solution to the aforementioned problems regarding antigen authenticity. Particular embodiments are directed toward developing native Env trimer immunogens and new methods to eliminate forms of non-functional Env, allowing development of pure authentic Env trimers as vaccines for the first time. To address this need for developing authentic immunogens that induce the production of neutralizing antibodies to immunodeficiency virus (e.g. HIV-1, HIV-2, SIV) in a vaccinated subject, several embodiments of the present invention relate to native trimers in a pure form that elicit effective nAb responses. A variety of embodiments relate to methods of selecting neutralizing antibodies from B cell repertoires of infected or vaccinated subjects and methods of vaccinating a subject against an immunodeficiency virus with the inventive immunogens.

In one embodiment, an immunogenic composition includes a virus-like particle having a surface with substantially only immunodeficiency virus trimeric gp120/gp41 Env protein bound to it and is capable of inducing production of neutralizing antibodies in a subject administered the composition. In one aspect, gp120/gp41 Env trimers includes a trimer of gp120/gp41 heterodimers that emerge from cleavage of a gp160 precursor immediately following an arginine or lysine residue at position 511 (LAI strain of Env numbering). In another aspect, the trimeric gp120/gp41 Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules.

In a further aspect of the previous embodiment, the surface of the immunogenic composition substantially lacks uncleaved gp160 Env protein decorated exclusively with high mannose glycans, termed gp160ER. In another aspect, the high mannose glycan has a molecular mass of about 1.5 kDa. In another aspect, the high mannose glycan is Man5GlcNac2, Man6GlcNac2, Man7GlcNac2, Man8GlcNac2 or Man9GlcNac2 (where Man=mannose and GlcNac=N-acetylglucosamine). In a further aspect, one high mannose glycan is linked to Asparagine 276 (N276, LAI numbering) of uncleaved gp160ER. In another aspect, gp160 has not undergone proteolytic processing immediately following residue 511 (using LAI numbering) of gp120 into gp120 and gp41 subunits. In the same aspect, the gp160 precursor is also not cleaved after the arginine or lysine at residue 504 of gp120 (using LAI Env numbering). In the same aspect, gp160 comprising gp120 and gp41 covalently linked. In yet another aspect, the surface of the immunogenic composition substantially lacks mature uncleaved gp160, decorated with a mixture of high mannose and complex glycans.

In yet another aspect of the previous embodiment, the virus-like particle further substantially lacks gp41 “stumps” bound to its surface in which gp41 is unlinked to gp120.

In another aspect of the previous embodiment, the immunodeficiency virus is HIV-1. In the same aspect, HIV-1 is selected from any group consisting of clades A, B, C, D, CRF01_AE, CRF02_AG, F1, F2, G, H, J, K N, O, P, U and inter-clade recombinant versions thereof.

In a further aspect of the previous embodiment, the immunodeficiency virus is HIV-2 or SIV.

In one aspect of the aforementioned embodiment, the trimeric Env protein includes 3 copies of noncovalently-associated gp120 and gp41. In one aspect, the gp120/gp41 trimer is a mutant having a disulfide linkage between gp120 and gp41 between residues 501 of gp120 and 605 of gp41 (LAI Env numbering).

In a further aspect of the previous embodiment, the composition further includes an adjuvant such as Ribi, QS21, Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, and APRIL, C3d.

In one aspect of the previous embodiment, the virus-like particle surface includes a plurality of trimeric gp120/gp41 Env proteins having different peptide sequences.

In another embodiment, an immunogenic composition includes immunodeficiency virus soluble trimeric gp120/gp41 Env protein and is capable of inducing production of neutralizing antibodies against the immunodeficiency virus in a subject administered the composition.

In one aspect, the trimeric gp120/gp41 Env protein has an outer domain receptor binding site glycosylated with complex glycan. In the same aspect, the complex glycan has a molecular mass of about 3 kDa. In another aspect, the complex glycan comprises more than 2 GlcNac molecules.

In a further aspect of the previous embodiment, the immunogenic composition substantially lacks soluble monomeric or oligomeric uncleaved gp160ER Env protein glycosylated with high mannose glycan, or mature uncleaved gp160, decorated with a mixture of high mannose and complex glycans. In one aspect, the high mannose glycan has a molecular mass of about 1.5 kDa. In another aspect, the high mannose glycan is Man5-9GlcNac2. In a further aspect, a high mannose glycan is linked to Asparagine 276 (N276) of the uncleaved gp160ER. In yet another aspect, uncleaved gp160 comprises of covalently linked gp120 and gp41.

In yet another aspect of the previous embodiment, the immunogenic composition further substantially lacks soluble gp41 “stumps” in which gp41 is unlinked to gp120.

In another aspect of the previous embodiment, the immunodeficiency virus is HIV-1. In the same aspect, HIV-1 is selected from the group consisting of clade A, B, C, D, CRF01_AE, CRF02_AG, F1, F2, G, H, J, K N, O, P, or U.

In a further aspect of the previous embodiment, the immunodeficiency virus is HIV-2 or SIV.

In one aspect of the aforementioned embodiment, the trimeric Env protein includes three copies of non-covalently-associated gp120 and gp41. Here, the gp160 precursor is cleaved after the residue (usually arginine or lysine at residue 511 of gp120, using LAI Env numbering) to make gp120/gp41 complexes. In the same aspect, the gp160 precursor may also be cleaved after the arginine or lysine at residue 504 of gp120, using LAI Env numbering). In one aspect, the gp120/gp41 trimers include a mutation that introduces a disulfide linkage between gp120 and gp41. This disulfide is formed by novel cysteines, usually inserted at residues 501 of gp120 and 605 of gp41 (LAI numbering).

In a further aspect of the previous embodiment, the composition further includes an adjuvant such as Ribi, QS21, and Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, and APRIL, C3d.

In one aspect of the previous embodiment, the soluble gp120/gp41 trimeric Env protein includes different peptide sequences represented by mutation or genetic clade variation.

In another embodiment, an immunogenic composition includes a microparticle having a surface that has substantially only immunodeficiency virus trimeric gp120/gp41 Env protein bound thereto and is capable of inducing production of neutralizing antibodies against the immunodeficiency virus in a subject administered the composition.

In one aspect, the trimeric Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules. However, in another aspect, Env may be produced in cell lines that are incapable of converting complex glycans into high mannose glycans, e.g. glucosylaminotransferase I deficient 293S cells. Thus, in this case, all glycans are similar, but gp120/gp41 trimers differ from uncleaved gp160 in being proteolytically processed after the arginine or lysine at residue 511 of gp120 (LAI numbering) and/or the arginine or lysine at residue 504 (LAI numbering), and are also substantially uniformly oligomeric. Uncleaved gp160s are not processed after residues 504 or 511 of gp120 and may be monomeric, dimeric, trimeric, tetrameric or other higher order complexes.

In a further aspect of the previous embodiment, the surface of the immunogenic composition substantially lacks uncleaved gp160ER glycosylated with high mannose glycan, or mature uncleaved gp160, decorated with a mixture of high mannose and complex glycans. In one aspect, the high mannose glycan has a molecular mass of about 1.5 kDa. In another aspect, the high mannose glycan is Man5-9GlcNac2. In a further aspect, the high mannose glycan is linked to Asparagine 276 (N276) of gp160ER. In yet another aspect, the uncleaved gp160 comprises of covalently linked gp120 and gp41.

In yet another aspect of the previous embodiment, the virus-like particle further substantially lacks gp41 “stumps” bound to its surface in which gp41 is unlinked to gp120.

In another aspect of the previous embodiment, the immunodeficiency virus is HIV-1. In the same aspect, HIV-1 is selected from the group consisting of clade A, B, C, D, CRF01_AE, CRF02_AG, F1, F2, G, H, J, K N, O, P, or U.

In a further aspect of the previous embodiment, the immunodeficiency virus is HIV-2 or SIV.

In one aspect of the aforementioned embodiment, the trimeric Env protein includes three copies of non-covalently-associated gp120 and gp41. In one aspect, the trimeric gp120/gp41 is a mutant having a disulfide linkage between residue 501 of gp120 and residue 605 of gp41 (LAI Env numbering).

In a further aspect of the previous embodiment, the composition further includes an adjuvant such as Ribi, QS21, and Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B (CTB; and its derivatives), ISCOMS, saponins, TDM, CWS emulsion, poly I:C, virosomes, alum, alhydrogel, CD40L, BAFF, and APRIL, C3d.

In one aspect of the previous embodiment, the virus-like particle surface includes a plurality of trimeric gp120/gp41 Env proteins having different peptide sequences.

In another embodiment, a method of making a vaccine against an immunodeficiency virus includes obtaining a plurality of immunodeficiency virus-like particles having a surface with trimeric gp120/gp41 Env protein and uncleaved gp160 Env protein, and contacting the virus-like particles with enzyme(s) that substantially and selectively remove the uncleaved gp160 Env protein from the surface to generate purified virus-like particles having a surface including substantially only trimeric gp120/gp41 Env protein bound to it.

In one aspect of the previous embodiment, enzymes may include at least one glycosidase and at least one protease or may include proteases alone (no glycosidase). In another aspect, the at least one glycosidase is any of endoglycosidase H (endo H), endo F1, PNGase F, neuraminidase, and mannosidase. In a preferred aspect, at least one glycosidase includes endo H. In the same aspect, about 0.01 to about 10,000 units of endo H are contacted per about 10 ng Env equivalent of the plurality of virus-like particles. In the same aspect, endo H is contacted with said virus-like particles for about 0.1 to 100 hours.

In another aspect of the previous embodiment, a single enzyme (e.g. a single protease) or class of enzyme (e.g. a plurality of proteases) may in some cases be used. In one aspect, the single protease may be chymotrypsin, trypsin, pepsin, elastase, papain, subtilisin, cathepsin C, pyroglutamate aminopeptidase, plasmin, furin, proteinase K and bromelain. In the same aspect, about 0.01 to about 1,000 units of chymotrypsin or other protease are contacted per about 10 ng Env equivalent of the plurality of virus-like particles. In the same aspect, chymotrypsin is contacted with said virus-like particles for about 0.1 to 100 hours.

In another aspect, the protease is any of chymotrypsin, trypsin, pepsin, elastase, papain, subtilisin, cathepsin C, pyroglutamate aminopeptidase, plasmin, proteinase K and bromelain. In a preferred embodiment, at least one protease is chymotrypsin. In the same aspect, about 0.01 to about 1,000 units of chymotrypsin are contacted per about 10 ng Env equivalent of said plurality of virus-like particles. In the same aspect, chymotrypsin is contacted with said virus-like particles for about 0.1 to 100 hours.

In a further aspect of the previous embodiment, the virus-like particles are in some instances first contacted with said glycosidase before contacted with said protease.

In an additional aspect, the method further includes contacting said virus-like particle with non-neutralizing antibody against Env that selectively binds and enhances removal of uncleaved gp160ER, mature gp160 and/or gp41 stumps by the enzyme(s). In the same aspect, the non-neutralizing antibody is monoclonal.

In a further aspect of the embodiment, the method also includes contacting the virus-like particle with a concentration of paraformaldehyde effective to crosslink and enhance removal of gp160ER by the enzyme(s) without substantially altering the conformation of the trimeric gp120/gp41 Env protein.

In one aspect of the previous embodiment, the trimeric gp120/gp41 Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules.

In a further aspect of the previous embodiment, the surface of the immunogenic composition substantially lacks uncleaved gp160 Env protein glycosylated with high mannose glycan, or mature uncleaved gp160, decorated with a mixture of high mannose and complex glycans. In one aspect, the high mannose glycan has a molecular mass of about 1.5 kDa. In another aspect, the high mannose glycan is Man5-9GlcNac2. In a further aspect, one of the high mannose glycans on gp160ER is linked to Asparagine 276 (N276) of uncleaved gp160ER. In yet another aspect, uncleaved gp160 comprises of covalently linked gp120 and gp41.

In yet another aspect of the previous embodiment, the virus-like particle further substantially lacks gp41 “stumps” bound to its surface in which gp41 is unlinked to gp120.

In another aspect of the previous embodiment, the immunodeficiency virus is HIV-1. In the same aspect, HIV-1 is selected from the group consisting of clade A, B, C, D, CRF01_AE, CRF02_AG, F1, F2, G, H, J, K N, O, P, or U.

In a further aspect of the previous embodiment, the immunodeficiency virus is HIV-2 or SIV.

In one aspect of the aforementioned embodiment, the trimeric Env protein includes three copies of non-covalently-associated gp120 and gp41. In one aspect, the 120/gp41 trimer is a mutant having a disulfide linkage between gp120 and gp41.

In a further aspect of the previous embodiment, the composition further includes an adjuvant such as Ribi, QS21, Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B (CTB; and its derivative), ISCOMS, saponins, TDM, CWS emulsion, poly I:C, virosomes, alum, alhydrogel, CD40L, BAFF, and APRIL, C3d.

In one aspect of the previous embodiment, the virus-like particle surface includes a plurality of trimeric gp120/gp41 Env proteins having different peptide sequences.

In one embodiment, a method of making a vaccine against an immunodeficiency virus includes providing a producer cell expressing immunodeficiency virus trimeric gp120/gp41 Env protein and uncleaved gp160 Env protein on its surface; contacting the producer cell with a concentration of an extraction agent effective to release soluble trimeric gp120/gp41 and uncleaved gp160 Env protein from the surface; and contacting the soluble trimeric gp120/gp41 and uncleaved gp160 Env protein usually with enzyme(s) that substantially and selectively remove the soluble uncleaved gp160 Env protein to generate a composition having substantially only soluble trimeric gp120/gp41 Env protein. In another embodiment, one protease enzyme may be sufficient to substantially and selectively remove the soluble uncleaved gp160 Env protein to generate a composition having substantially only soluble trimeric gp120/gp41 Env protein.

In one aspect, the producer cell is an immunodeficiency virus-like particle expressing wild-type Env protein, an immunodeficiency virus-like particle expressing mutant Env protein having a disulfide linkage between gp120 and gp41, a transfected cell ectopically expressing wild-type or mutant Env protein, a virally transduced cell ectopically expressing wild-type or mutant Env protein, or an immunodeficiency virus-infected cell.

In another aspect, the extraction agent is a detergent. In the same aspect, the detergent is non-ionic. In the same aspect, the non-ionic detergent is Triton. Further within that aspect, the concentration of Triton is about 0.01% to about 1%. In the same aspect, the concentration of Triton is about 0.15%.

In one aspect, the enzymes may include at least one glycosidase and at least one protease. In the same aspect, the at least one glycosidase is any of endo H, endo F1, F2, F3, or D, PNGase F, galactosidase, O-glycanase, fucosidase, neuraminidase, and mannosidase. In a preferred embodiment, the at least one glycosidase includes endo H. In the same aspect, about 0.01 to about 10,000 units of endo H are contacted per about 10 ng of soluble Env protein. Further in the same aspect, wherein said units of endo H are contacted with said soluble Env protein for about 0.1 to 100 hours. In the same aspect, the protease is any of chymotrypsin, trypsin, pepsin, elastase, papain, subtilisin, cathepsin C, pyroglutamate aminopeptidase, plasmin, proteinase K and bromelain. In a preferred aspect, the at least one protease includes chymotrypsin. Still further in the same aspect, about 0.01 to about 1,000 units of chymotrypsin are contacted per about 10 ng of soluble Env protein. In the same aspect, chymotrypsin is contacted with the soluble Env protein for about 0.1 to 100 hours. Alternatively, 1 μg each of trypsin, chymotrypsin, subtilisin and proteinase is contacted with soluble Env protein for about 0.1 to 100 hours.

In another aspect, the soluble Env protein is first contacted with said glycosidase before contact with said protease. Alternatively, soluble Env protein is contacted with protease alone.

In another aspect of the previous embodiment, a single enzyme (e.g. a single protease) or class of enzyme (e.g. a plurality of proteases) may in some cases be used. In one aspect, the single protease may be chymotrypsin, trypsin, pepsin, elastase, papain, subtilisin, cathepsin C, pyroglutamate aminopeptidase, plasmin, proteinase K and bromelain. In the same aspect, about 0.01 to about 1,000 units of chymotrypsin or other protease are contacted per about 10 ng Env equivalent of the plurality of virus-like particles. In the same aspect, chymotrypsin is contacted with said virus-like particles for about 0.1 to 100 hours. Alternatively, 1 μg each of trypsin, chymotrypsin, subtilisin and proteinase is contacted with soluble Env protein for about 0.1 to 100 hours.

In an additional aspect of the previous embodiment, the method further includes contacting the producer cell with non-neutralizing antibody against Env that selectively binds and enhances digestion of uncleaved gp160ER, mature gp160 and/or gp41 stumps by the said enzyme(s). In the same aspect, the non-neutralizing antibody is monoclonal.

In a further aspect, the method also includes contacting the producer cell with a concentration of paraformaldehyde effective to crosslink and enhance digestion of gp160ER, mature gp160 and gp41 stumps by said enzyme(s) without substantially altering the conformation of the trimeric gp120/gp41 Env protein.

In one aspect of the previous embodiment, the trimeric gp120/gp41 Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules. In one aspect, the trimeric Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules. However, in another aspect, Env may be produced in cell lines that are incapable of converting complex glycans into high mannose glycans, e.g. glucosylaminotransferase I deficient 293S cells. Thus, in this case, all glycans are similar, but gp120/gp41 trimers differ from uncleaved gp160 in being proteolytically processed after the arginine or lysine at residue 511 of gp120 (LAI numbering) and may also be proteolytically processed after the arginine or lysine at residue 504 of gp120 (LAI numbering) and are also uniformly oligomeric. Uncleaved gp160s are not processed after residues 504 or 511 of gp120 and may be monomeric, dimeric, trimeric, tetrameric or other high order complexes.

In a further aspect of the previous embodiment, the surface of the immunogenic composition substantially lacks uncleaved gp160ER glycosylated with high mannose glycan. In one aspect, the high mannose glycan has a molecular mass of about 1.5 kDa. In another aspect, the high mannose glycan is Man5-9GlcNac2. In a further aspect, the high mannose glycan is linked to Asparagine 276 (N276) of uncleaved gp160ER. In yet another aspect, the surface of the immunogenic composition also substantially lacks uncleaved mature gp160, decorated by a mixture of high mannose and complex glycans. In a further aspect, uncleaved gp160 is unprocessed and thus consists of covalently linked gp120 and gp41.

In yet another aspect of the previous embodiment, the virus-like particle further substantially lacks gp41 “stumps” bound to its surface in which gp41 is unlinked to gp120.

In another aspect of the previous embodiment, the immunodeficiency virus is HIV-1. In the same aspect, HIV-1 is selected from the group consisting of clade A, B, C, D, CRF01_AE, CRF02_AG, F1, F2, G, H, J, K N, O, P, or U.

In a further aspect of the previous embodiment, the immunodeficiency virus is HIV-2 or SIV.

In one aspect of the aforementioned embodiment, the trimeric Env protein includes three copies of non-covalently associated gp120 and gp41 resulting from cleavage of gp160 immediately after the lysine or arginine at residue 511 (LAI numbering) and may also be proteolytically processed after the arginine or lysine at residue 504 of gp120 (LAI numbering). In one aspect, the gp120/gp41 trimers have a mutation that introduces a disulfide linkage between residues 501 of gp120 and residue 605 of gp41 (LAI numbering).

In a further aspect of the previous embodiment, the composition further includes an adjuvant such as Ribi, QS21, and Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, APRIL, and C3d.

In yet a further aspect, the method includes purifying the soluble gp120/gp41 trimeric Env protein by chromatography. In the same aspect, soluble gp120/gp41 trimeric Env protein is purified by lectin chromatography. Further in the same aspect, the soluble gp120/gp41 trimeric Env protein is purified by size exclusion chromatography.

In an additional aspect of the previous embodiment, the composition comprises a polyvalent mixture of soluble trimeric gp120/gp41 Env protein having different peptide sequences represented by mutation or genetic clade variation.

In one embodiment, a method of making a vaccine against an immunodeficiency virus includes obtaining a producer cell expressing immunodeficiency virus trimeric gp120/gp41 Env protein and uncleaved gp160 protein on its surface; contacting the producer cell with enzyme(s) that substantially and selectively remove the uncleaved gp160 Env protein to generate a treated cell expressing substantially only gp120/gp41 trimeric Env protein; and contacting the treated cell with a concentration of an extraction agent effective to release the trimeric gp120/gp41 Env protein from the surface to generate soluble trimeric gp120/gp41 Env protein suitable for use as a vaccine.

In one aspect, the producer cell is an immunodeficiency virus-like particle expressing wild-type Env protein, an immunodeficiency virus-like particle expressing mutant Env protein having a disulfide linkage between residue 501 of gp120 and residue 605 of gp41 (LAI numbering), a transfected cell ectopically expressing wild-type or mutant Env protein, a virally transduced cell ectopically expressing wild-type or mutant Env protein, or an immunodeficiency virus-infected cell.

In another aspect, the extraction agent is a detergent. In the same aspect, the detergent is non-ionic. In the same aspect, the non-ionic detergent is Triton. Further within that aspect, the concentration of Triton is about 0.01% to about 1%. In the same aspect, the concentration of Triton is about 0.15%.

In one aspect, the enzymes may include at least one glycosidase and at least one protease. In the same aspect, the at least one glycosidase is any of endo H, endo F1, PNGase F, neuraminidase, and mannosidase. In a preferred embodiment, the at least one glycosidase includes endo H. In the same aspect, about 0.01 to about 10,000 units of endo H are contacted per about 10 ng of soluble Env protein. Further in the same aspect, wherein said units of endo H are contacted with said soluble Env protein for about 0.1 to 100 hours. In the same aspect, the protease is any of chymotrypsin, trypsin, pepsin, elastase, papain, subtilisin, cathepsin C, pyroglutamate aminopeptidase, plasmin, proteinase K and bromelain. In a preferred aspect, the at least one protease includes chymotrypsin. Still further in the same aspect, about 0.01 to about 1,000 units of chymotrypsin are contacted per about 10 ng of soluble Env protein. In the same aspect, chymotrypsin is contacted with the soluble Env protein for about 0.1 to 100 hours.

In another aspect, the producer cell is first contacted with the glycosidase before contact with the protease.

In another aspect of the previous embodiment, a single enzyme (e.g. a single protease) or class of enzyme (e.g. a plurality of proteases) may in some cases be used. In one aspect, the single protease may be chymotrypsin, trypsin, pepsin, elastase, papain, subtilisin, cathepsin C, pyroglutamate aminopeptidase, plasmin, proteinase K and bromelain. In the same aspect, about 0.01 to about 1,000 units of chymotrypsin or other protease are contacted per about 10 ng Env equivalent of the plurality of virus-like particles. In the same aspect, chymotrypsin is contacted with said virus-like particles for about 0.1 to 100 hours.

In a further aspect, the method includes contacting said producer cell with non-neutralizing antibody against Env that selectively binds and enhances digestion of uncleaved gp160 Env protein by said enzyme(s). In the same aspect, the non-neutralizing antibody is monoclonal.

In an additional aspect, the method further includes contacting the producer cell with a concentration of paraformaldehyde effective to crosslink and enhance digestion of uncleaved gp160 by the enzyme(s) without substantially altering the conformation of the trimeric gp120/gp41 Env protein.

In one aspect of the previous embodiment, the trimeric gp120/gp41 Env protein has an outer domain receptor binding site glycosylated with complex glycan. In the same aspect, the complex glycan has a molecular mass of about 3 kDa. In another aspect, the complex glycan comprises more than 2 N-acetylglucosamine molecules. In yet another aspect, gp120/gp41 trimers consist of non-covalently-associated gp120 and gp41 subunits, resulting from cleavage of a gp160 precursor.

In a further aspect of the previous embodiment, the surface of the immunogenic composition substantially lacks uncleaved gp160ER glycosylated with high mannose glycan. In one aspect, the high mannose glycan has a molecular mass of about 1.5 kDa. In another aspect, a high mannose glycan may be Man5-9GlcNac2. In a further aspect, a high mannose glycan is linked to Asparagine 276 (N276) of uncleaved gp160ER. In yet another aspect, the surface of the immunogenic composition substantially lacks uncleaved mature gp160 decorated by a mixture of high mannose and complex glycans. In a further aspect, uncleaved gp160 is unprocessed and thus consists of covalently linked gp120 and gp41.

In yet another aspect of the previous embodiment, the virus-like particle further substantially lacks gp41 “stumps” bound to its surface in which gp41 is unlinked to gp120.

In another aspect of the previous embodiment, the immunodeficiency virus is HIV-1. In the same aspect, HIV-1 is selected from the group consisting of clade A, B, C, D, CRF01_AE, CRF02_AG, F1, F2, G, H, J, K N, O, P, or U.

In a further aspect of the previous embodiment, the immunodeficiency virus is HIV-2 or SIV.

In one aspect of the aforementioned embodiment, the trimeric Env protein includes three copies of non-covalently-associated gp120 and gp41. In one aspect, the gp120/gp41 trimers include mutations that introduce a disulfide linkage between residue 501 of gp120 and residue 605 of gp41.

In a further aspect of the previous embodiment, the composition further includes an adjuvant such as Ribi, QS21, and Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, and APRIL, C3d.

In yet a further aspect, the method includes purifying the soluble trimeric gp120/gp41 Env protein by chromatography. In the same aspect, soluble trimeric gp120/gp41 Env protein is purified by lectin chromatography. Further in the same aspect, the soluble trimeric gp120/gp41 Env protein is purified by size exclusion or ion exchange chromatography.

In an additional aspect of the previous embodiment, the composition comprises a polyvalent mixture of soluble trimeric gp120/gp41 Env protein having different peptide sequences represented by mutation or genetic clade variation.

In one embodiment, a method of selecting neutralizing antibodies against Env protein includes sorting memory B cells from an immunodeficiency virus-infected subject, contacting antibodies produced by the sorted B cells with soluble trimeric gp120/gp41 or a particle having substantially only trimeric gp120/gp41 Env protein bound thereto, and identifying the B cells that produce neutralizing antibodies against said trimeric gp120/gp41.

In one embodiment, a method of immunizing a mammal against an immunodeficiency virus includes administering an effective amount of the immunogenic composition of any of the embodiments described herein to the mammal sufficient to induce production of neutralizing antibodies against the immunodeficiency virus in the mammal. In one aspect, the method includes sequentially administering an effective amount of the immunogenic composition of any of the embodiments described herein in which the trimeric gp120/gp41 Env proteins sequentially administered have different amino acid sequences or belong to different genetic clades.

### BRIEF DESCRIPTION OF THE DRAWINGS

FIG. 1 is a model contrasting the forms of native gp120/gp41 Env trimers, uncleaved gp160ER, mature gp160 and gp41 stumps.

FIG. 2 is a cartoon depicting schematically, the two major domains of gp120 in gp120/gp41 trimers, gp160ER and mature gp160, namely the neutralizing and silent faces. These are decorated by either high mannose or complex glycans that dictate the susceptibility of each species to glycosidase and protease digestion.

FIGS. 3A-B are reducing SDS-PAGE Western blots of SOS-VLPs produced in 293T or GnTI-cells and gp120 produced in CHO cells. Samples were treated with or without endo H after denaturation.

FIGS. 4A-D are an analysis of the effect of gp120/gp41 cleavage site mutations on trimer formation by BN-PAGE and on cleavage by SDS-PAGE. The infectivity of each mutant was also examined.

FIGS. 5A-B are BN-PAGE Western blots of WT-VLPs and SOS-VLPs pre-treated or not with endo H.

FIGS. 6A-B are BN-PAGE Western blots of SOS-VLPs, UNC SOS-VLPs and other mutants, using mAb 2G12 in shift assays.

FIG. 7 is an SDS-PAGE Western blot showing the effects of glycosidase and protease digests on UNC WT-VLPs.

FIG. 8 is a BN-PAGE Western blot analysis of WT-VLPs pre-treated or not with endo H followed by digestion with various proteases.

FIG. 9 is a BN-PAGE Western blot analysis of SOS-VLPs pre-treated or not with endo H followed by digestion with various proteases.

FIG. 10 is a BN-PAGE Western blot analysis of the stability of WT-VLP trimers following digestion.

FIG. 11 is a BN-PAGE Western blot analysis of various JR-FL VLPs pre-treated or not with endo H followed by digestion with various proteases.

FIG. 12 is a BN-PAGE Western blot of Env-VLPs of differing neutralization sensitivities from different genetic backgrounds and clades treated with or without the combination of endo H and chymotrypsin, trypsin, subtilisin and proteinase K.

FIG. 13 is a bar chart showing the survival of VLP infectivity following enzyme digests.

FIG. 14 is a BN-PAGE Western blot of VLPs solubilized in triton before endo H and protease treatments.

FIG. 15 is a bar graph showing the effect of endo H/chymotrypsin digests on mAb capture of SOS-VLPs.

FIGS. 16A-C are BN-PAGE Western blots of VLPs A) untreated or B) treated with a mixture of trypsin, chymotrypsin, proteinase K and subtilisin, and then shifted with various mAbs.

FIG. 17 is a schematic of a method for producing a VLP with only authentic gp120/gp41 Env trimers.

FIG. 18 is a schematic of a method for producing VLPs bearing mutant forms of Env or Env clones from different genetic backgrounds and clades.

FIG. 19 is a schematic of a method for producing soluble immunogenic gp120/gp41 Env trimer from a variety of sources.

FIG. 20 is a digest of the JR-FL E168K mutant (in WT background). Protease digests result in very clean trimers (lane 3)

FIGS. 21A-B are graphs of ELISA showing the binding of various neutralizing and non-neutralizing monoclonal antibodies to JR-FL WT-VLPs with an E168K after digestion with proteases trypsin, chymotrypsin, proteinase K and subtilisin overnight in thin walled tubes.

### DETAILED DESCRIPTION

HIV-1 has evolved sophisticated mechanisms to shield sites on the functional native form of Env from antibodies. These shielded sites are exposed on other, aberrant forms of Env that appear to function as decoys, disfavoring antibody responses to the native form of Env. Improved antibody responses against native Env might be possible if native Env can be isolated in the absence of decoy Env antigens. Not to be bound to a particular theory, various present embodiments involve eliminating the aberrant forms of Env, so that, hypothetically at least, B cell responses can refocus on native, authentic forms of Env, resulting in the production of neutralizing antibodies (64, 82).

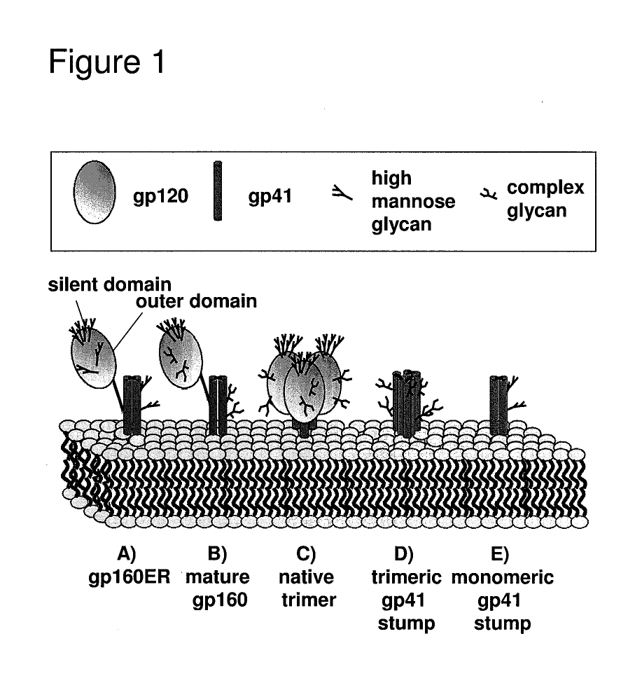

Strategies to eliminate aberrant Env may benefit from a better understanding of its nature and how it differs from the functional form of Env. The native, functional form of Env exposes neutralizing epitopes, while non-neutralizing epitopes are occluded. The native, functional form of Env includes three gp120 moieties that are non-covalently associated with three underlying gp41 components in a configuration whose fusion potential is triggered by engagement with cell surface receptors CD4 and a chemokine receptor, usually CCR5. The native, functional form of Env is referred to herein as the native gp120/gp41 trimer. FIG. 1 depicts various forms of particulate Env including native gp120/gp41 trimers (FIG. 1C) and various aberrant forms of Env that include two forms of gp160 (FIG. 1A, B) and gp41 stumps (FIG. 1D, E).

Native gp120/gp41 trimers (FIG. 1C) differ from gp160 (FIG. 1A, B) in that they are the product of gp160 precursor processing into gp120 surface and gp41 transmembrane subunits during cellular synthesis in the trans-Golgi network by the actions of furin or related proteases. In contrast, gp160 is uncleaved, such that the gp120 and gp41 subunits remain covalently-associated. This affects their topology compared to the native gp120/gp41 trimers, the latter being substantially more compact. As depicted in FIG. 1, complex outer domain glycans line the receptor binding sites in the native trimer (FIG. 1C) and mature gp160 (FIG. 1B). These are replaced by smaller Man5GlcNac2 in gp160ER (FIG. 1A). The high mannose glycans of the silent domain are identical or near identical in mature trimers and both forms of gp160 (FIG. 1A-C). Mature gp160 has a similar glycan distribution compared to the native Env trimer, but resembles gp160ER morphologically, with a non-native gp120-gp41 covalent association. Uncleaved gp160s may be monomeric or oligomeric. Only monomeric forms are shown in FIG. 1A and FIG. 1B. Oligomers may form due to the aggregation of hydrophobic domains of gp41, such as the fusion peptide. These uncleaved gp160 precursor proteins do not mediate virus-cell fusion. As vaccine immunogens, uncleaved gp160s offer no advantages over gp120 monomer, which is also a poor mimic of the gp120/gp41 trimer. Other forms of aberrant Env are the gp41 stumps (FIG. 1D, E), that remain when gp120 dissociates from gp41.

Gp120 shedding can be overcome by introducing a gp120-gp41 disulfide bridge, termed the SOS mutant (10). SOS mutant VLPs (SOS-VLPs) can engage receptors and full fusion occurs upon the addition of a reducing agent to break the disulfide bridge (1, 8, 9, 21). SOS-VLPs exhibit the same resistance profile as wild type WT-VLPs, suggesting that essential trimer topology is preserved (21). Despite this advance, the failure of SOS-VLPs to induce improved neutralizing responses hinted at the presence of still other forms of non-functional Env (20). In this regard, blue native PAGE (BN-PAGE) analysis of Env liberated from SOS-VLPs resolved two bands: authentic trimers and an aberrant monomer (65). Until recently, it was thought that the latter monomer was a by-product of native gp120/gp41 trimer dissociation. However, it has now been found to consist of two different forms of uncleaved gp160, namely gp160ER and mature gp160 (FIG. 1A, B). As mentioned above, both gp160s differ from mature gp120/gp41 trimers in that the cleavage site between gp120 and gp41 in the gp160 precursor remains unprocessed, so that gp120 and gp41 are covalently bonded. The two gp160s differ from each other in that gp160ER (endoplasmic reticulum; FIG. 1A) is decorated exclusively with immature oligomannose glycans, while mature gp160 bears a mixture of high mannose and complex glycans like the native gp120/gp41 trimer (FIG. 1B). The two uncleaved gp160s are depicted as monomers in FIGS. 1A and B. However, they can also form multimers, including dimers, trimers, tetramers and higher order aggregates.

Considering the above developments, several embodiments provided herein relate to methods to selectively eliminate mature uncleaved gp160, uncleaved gp160ER, gp41 stumps and other aberrant forms of Env that do not resemble the gp120/gp41 trimer. Considering the distinct features of uncleaved gp160 and gp41 stumps (FIGS. 1A, B, D and E), compared to the native gp120/gp41 trimer (FIG. 1C), several embodiments are drawn to their greater sensitivity to enzyme digestion compared to the gp120/gp41 trimer. In several embodiments, digestion can be performed using one or more enzymes (e.g. a protease or a cocktail of proteases). In other embodiments, a two-step procedure is contemplated, in which the glycan shell of uncleaved gp160 is first dented by glycosidases, rendering it more vulnerable to proteases. In particular, the exclusively immature glycans of gp160ER provide opportunities for certain glycosidases (6). They are also smaller than complex glycans, implying an inherently thinner shell (6). Thus, in FIG. 1A, the primary receptor binding site is more accessible than it is on trimers (FIG. 1C), in part due to a smaller glycan at N276 (6). Conformation may also be a factor in selective digestion: while native Env gp120/gp41 trimers resist most glycosidases, proteases and non-neutralizing Abs (61), gp160 is generally more sensitive (19, 21, 28, 65, 81). In several embodiments provided herein, proteases alone can be effective without priming with glycosidases to selectively eliminate mature uncleaved gp160, uncleaved gp160ER, gp41 stumps and other aberrant forms of Env that do not resemble the gp120/gp41 trimer.

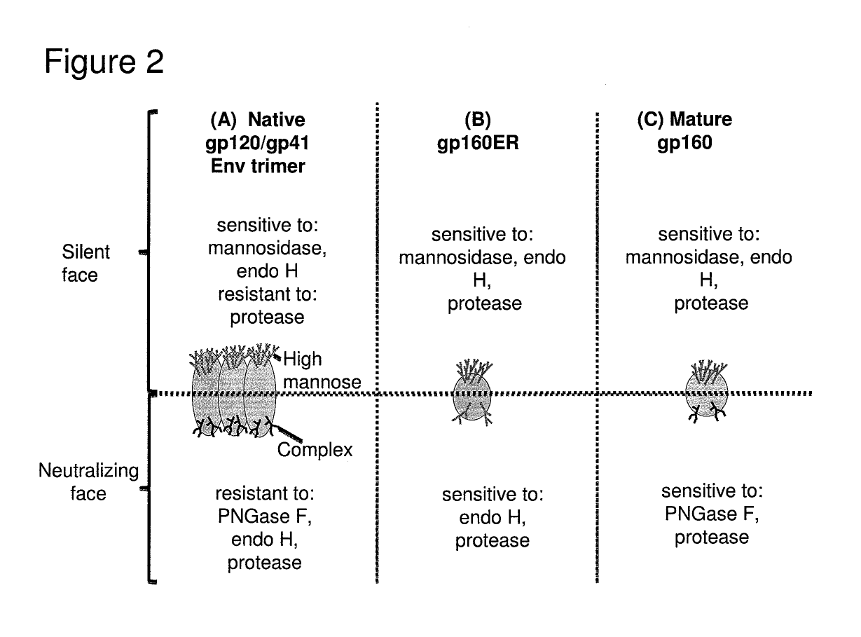

FIG. 2 shows a schematic comparison of native gp120/gp41 trimers, the two forms of gp160 and their inferred enzyme sensitivities. The gp160s (FIG. 2B, C) differ from gp120/gp41 trimers (FIG. 2A) in that they are uncleaved, which affects their conformation. Gp160 may be monomeric as shown, but can also form multimers. The gp120 subunits of each form of Env in FIG. 2 consist of a silent face (top) and neutralizing face (bottom). In all cases, the silent domain exhibits very tightly packed immature high mannose glycans. In contrast, the neutralizing face is decorated by less densely packed complex glycans in the gp120/gp41 trimers and mature gp160, and by less densely packed high mannose glycans in gp160ER. The enzyme-sensitivities of the silent and neutralizing faces of each form of Env are indicated in FIG. 2. The relative resistance of native trimer to enzymes, in particular proteases means that enzyme treatments can selectively remove all gp160, leaving behind the trimer.

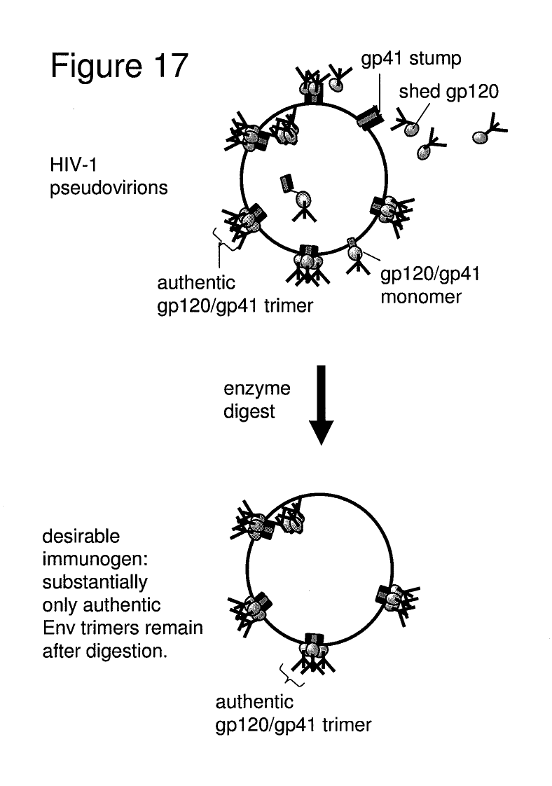

In several embodiments, the glycan shell is dented by priming with a glycosidase, which may expose the underlying protein for protease digestionresulting in VLPs on which trimers are presented in the absence of non-functional Env, as depicted in FIG. 17. Due to the less dense glycan packing, the neutralizing face (FIG. 2, lower section) can be a target for glycosidases. High mannose glycans are uniquely sensitive to endo H, since they are not fucosylated like complex glycans. In several embodiments, gp160ER can be removed by digesting the high mannose glycans of the neutralizing face glycans with endo H, followed by chymotrypsin. Gp120/gp41 trimers and mature gp160 are unaffected by this treatment. This leaves the problem of eliminating mature gp160. Without being bound by theory, given that mature gp160 shares the same glycans as gp120/gp41 trimers, a successful digestion procedure may depend more on the conformational differences between these two species that might render them differentially sensitive to enzymes. These two Env species differ in gp120/gp41 processing and their capacity to form compact trimers.

Several embodiments provided herein include digesting the compact high mannose glycans of the silent domain with combinations of mannosidases and endo H (FIG. 2). The silent domain of mature gp160 may then be sensitive to chymotrypsin. Alternatively, various embodiments target the outer domain complex glycans with glycosidases that include PNGase F, endoglycosidases F1, F2, F3, D, neuraminidase (sialidase), O-glycanase, galactosidase, and fucosidase, alone or in combination. Both approaches depend on the differential sensitivity of mature gp160 and gp120/gp41 trimers to glycosidase and/or protease digestion. Other approaches to augment the activities of these enzymes against mature gp160 contemplated herein include complexing with monoclonal antibodies that bind only mature gp160 and not gp120/gp41 trimers, thus fixing mature gp160 into a conformation that is more susceptible to enzyme digestion.

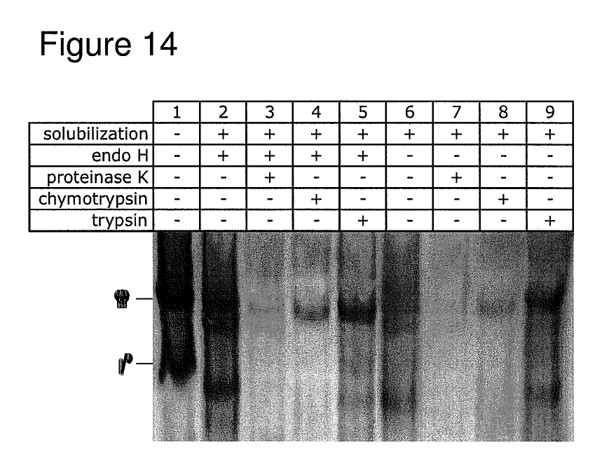

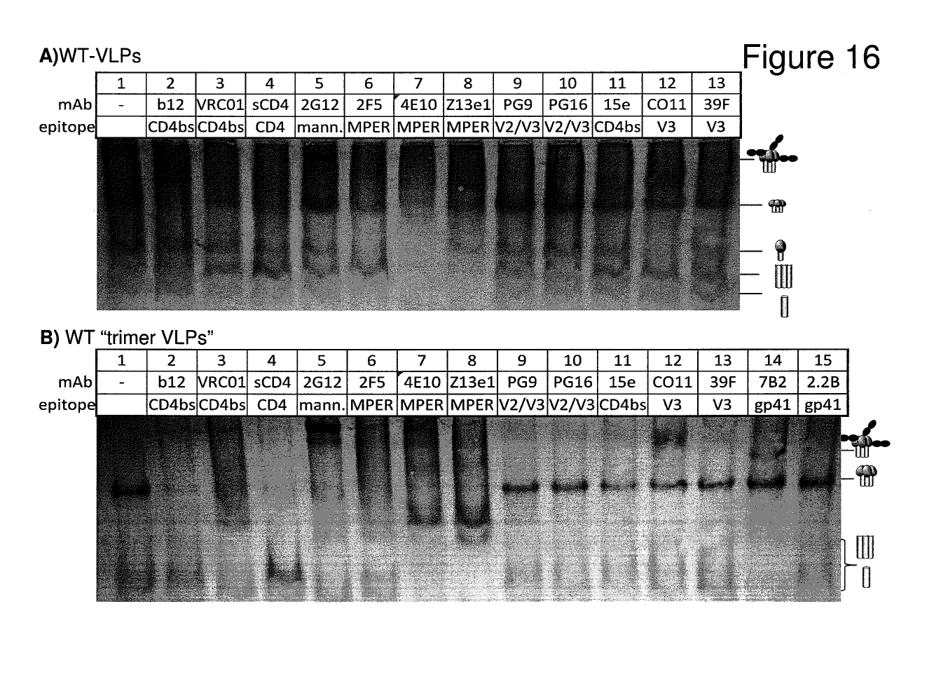

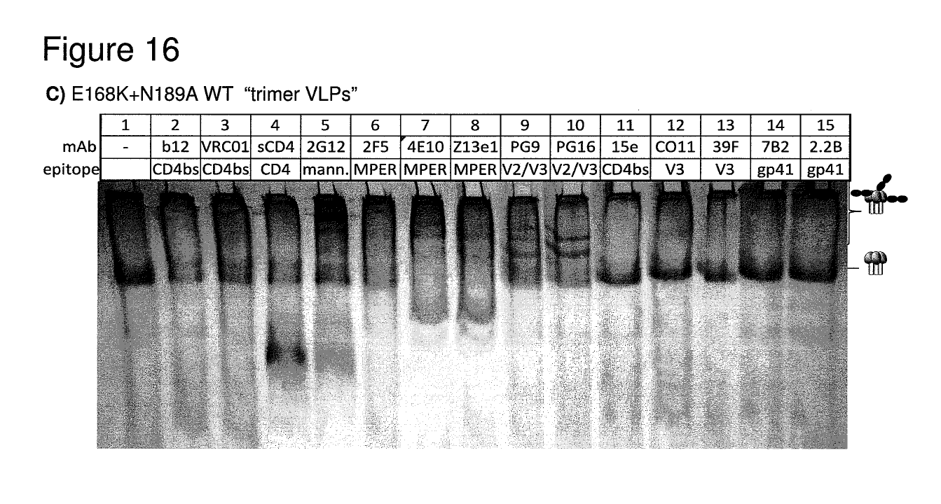

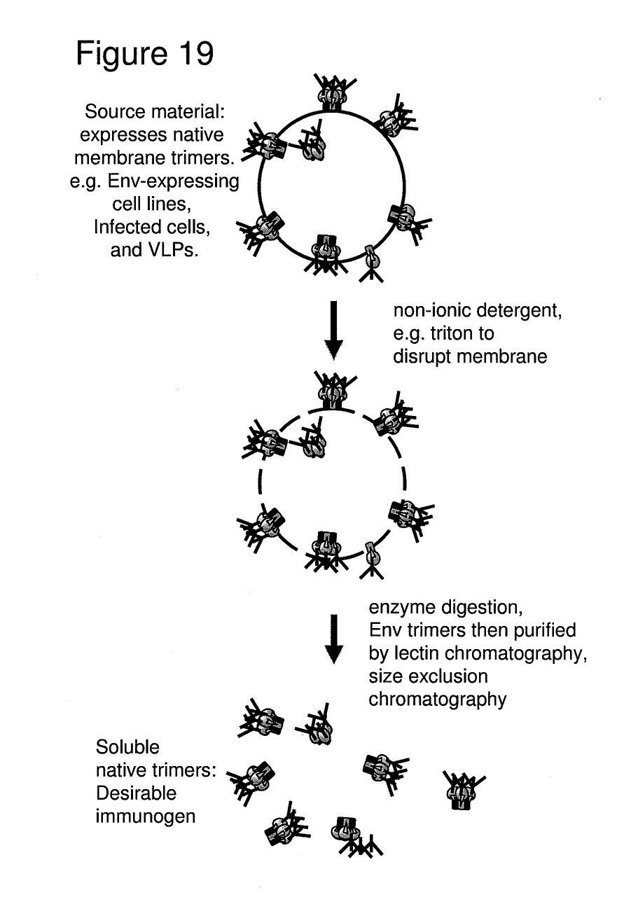

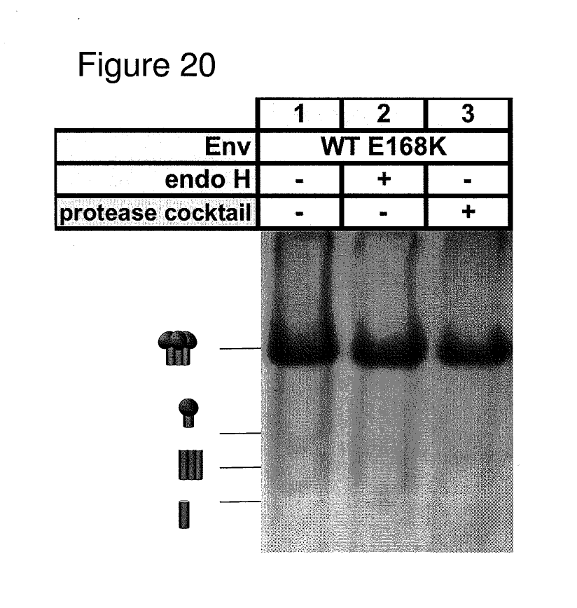

In some embodiments provided herein, endo H/chymotrypsin digests can remove gp160ER from JR-FL SOS-VLPs, leaving behind gp120/gp41 trimers and some mature gp160. When particles are solubilized, the same treatment removes both forms of gp160 (as shown in FIG. 14 and depicted schematically in FIG. 19). Without being bound by theory, this is consistent with the idea that isolating Env from membranes relaxes some of the constraints on enzyme digestion. In other embodiments provided herein, a combination of proteases (typsin, chymotrypsin, proteinase K and subtilisin) can be equally effective alone as when primed with endo H or other glycosidases. In one aspect, the use of thin walled tubes can be used to increase the efficiency of digests as shown in FIG. 16B, and FIG. 16C, and FIG. 20, which show that little or no aberrant Env is present, but trimers are still prominent.

Several embodiments provide that enzyme digests can select gp120/gp41 trimers from mixed sources. Various embodiments relate to compositions and methods involving these pure gp120/gp41 trimers to selectively elicit nAbs or to use as probes to isolate novel neutralizing monoclonal antibodies.

Several embodiments are drawn to pure particulate (e.g. VLP or microparticle) and soluble gp120/gp41 trimer immunogens, methods of making them, and methods of immunizing a subject, preferably a mammal, with the pure particulate and/or soluble gp120/gp41 trimer immunogens. In various embodiments, particles bearing substantially only gp120/gp41 Env trimers could be used as probes for B cell repertoires.

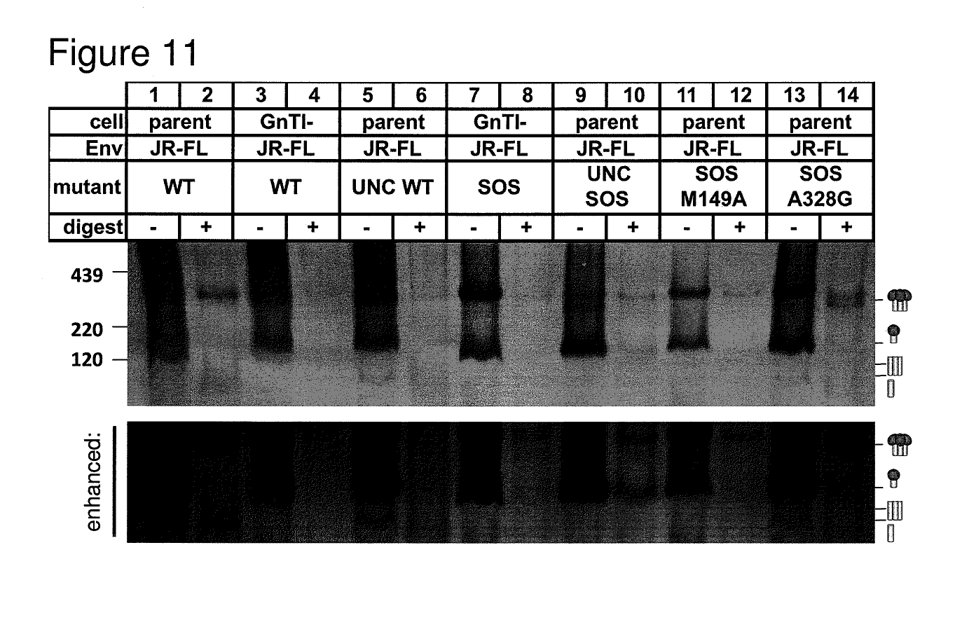

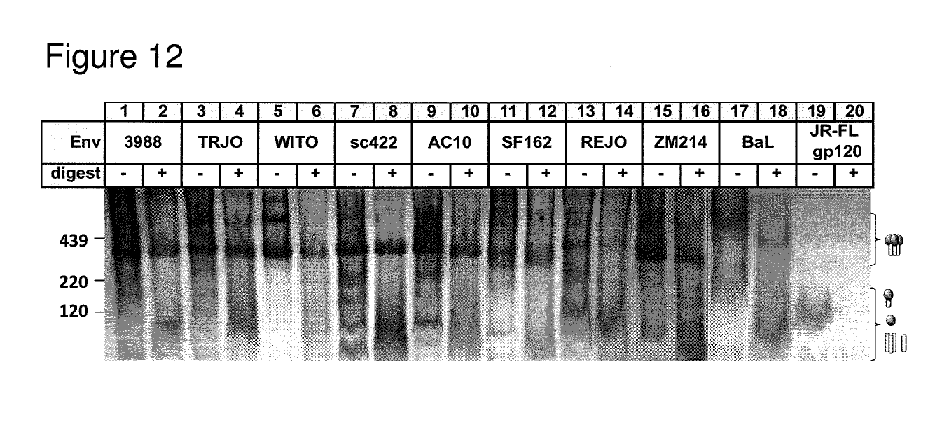

It will be understood that embodiments of the present invention encompass wild-type, mutant, or genetic variations of Env from different clades (FIGS. 11, 12 18 and 20). In several embodiments provided herein, the Env protein can be encoded by a nucleotide sequence or have an amino acid sequence including but not limited to any of the nucleotide or amino acid sequences of SEQ ID NOs: 1-46 provided herewith. Furthermore, the present embodiments can include but are not limited to Env sequences accessible in the online HIV Sequence Database: http://www.hiv.lanl.gov/components/sequence/HIV/search/search.html, which is herein incorporated by reference in its entirety for description of all nucleotide and amino acid sequences therein.

Env amino acid numbering as used herein refers to the LAI strain, but it will be understood that corresponding amino acids of Env variants which may have different amino acid numbering are encompassed by the embodiments of the present invention.

Embodiments of the present invention include variants of described Env sequences having about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% identity, or any number or range in between the aforesaid values, with respect to any length of described Env sequences. In several embodiments, the Env protein can have an amino acid sequence at least about 50%, 60%, 70%, 75%, 80%, 85%, 90%, 95%, 96%, 97%, 98%, or 99% identical to any of the known Env amino acid sequences available at the aforementioned online HIV Sequence Database and/or provided herewith in SEQ ID Nos: 1-46. In several embodiments, the Env protein can have an amino acid sequence encoded by a nucleotide sequence having at least about 50%, 60%, 70%, 75%, 80%, 85%, 90%, 95%, 96%, 97%, 98%, or 99% identity to any of the known Env nucleotide sequences available at the aforementioned online HIV Sequence Database and/or provided herewith in SEQ ID Nos: 1-46. In several embodiments, Env nucleotide sequences and amino acid sequences have at least about 50%, 60%, 70%, 75%, 80%, 85%, 90%, 95%, 96%, 97%, 98%, or 99% identity to any of the known Env nucleotide and amino acid sequences available at the aforementioned online HIV Sequence Database and/or SEQ ID Nos: 1-46, with respect to nucleotide or amino acid insertions, deletions, and/or substitutions (conservative or non-conservative).

A. VLPs Bearing Authentic gp120/gp41 Env Trimers as the Substantially Sole Form of Env

Several embodiments are drawn to an immunogenic virus-like particle (VLP) having a surface with substantially only immunodeficiency virus trimeric gp120/gp41 Env protein bound thereto. In other words, the immunogenic VLP surface substantially lacks uncleaved gp160 and/or gp41 unlinked to gp120 (also known as gp41 stumps). In other words, particles that lack all aberrant forms of Env, as exemplified in FIGS. 1A, B, D and E, but retaining intact gp120/gp41 Env trimers (FIG. 1C). As used herein, the term “substantially” is not limited to a particular quantity, ratio, or range. Thus, a VLP having a surface with substantially only gp120/gp41 Env trimer bound thereto has a quantity of gp120/gp41 Env trimer effective to induce a neutralizing antibody response in a subject, or a ratio of Env gp120/gp41 trimer to uncleaved gp160 and/or gp41 stump effective to induce a neutralizing antibody response in a subject. In various embodiments, the ratio of Env trimer to uncleaved gp160 on the surface of a VLP can be in the range of about 99:1 to about 1:99, including a range of about 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, and about 5:95. In one particular embodiment, a VLP has solely Env trimer on its surface (i.e. no uncleaved gp160 or gp41 stumps).

The trimeric gp120/gp41 Env protein on the surface of a VLP has a gp120 outer domain (neutralizing face) receptor binding site glycosylated with complex glycan (FIG. 2). The immunogenic VLPs substantially lack uncleaved Env protein gp160ER, which unlike trimeric gp120/gp41 Env glycoprotein, exhibits high mannose glycans (e.g. Man5-9GlcNac2) on the outer domain (FIG. 2B). Furthermore, the trimeric gp120/gp41 Env protein is relatively resistant to non-neutralizing monoclonal antibodies, glycosidase (e.g. endo H) digestion, and protease (e.g. chymotrypsin) digestion compared to uncleaved gp160ER. In several embodiments, methods are provided involving selective enzymatic digestion of gp160ER. Not limited to a particular theory, the high mannose (e.g. Man5-9GlcNac2) in the receptor binding site of uncleaved gp160ER exposes the underlying b12 binding patch, which contributes to endo H-sensitivity, which in turn primes sensitivity to protease, for example, chymotrypsin. Similar selective digestion methods may remove mature gp160 and gp41 stumps (FIGS. 1, 2).

Several embodiments of the present invention relate to methods of producing an immunogenic VLP having a surface with substantially only immunodeficiency virus trimeric gp120/gp41 Env protein bound thereto (FIG. 17). In one embodiment, the method includes obtaining a VLP having a surface expressing both trimeric gp120/gp41, uncleaved gp160 and gp41 stumps and contacting said VLP with one or more enzymes that substantially and selectively remove the aberrant Env (gp160ER, mature gp160 and gp41 stumps) from the surface, generating a purified virus-like particle having a surface with substantially only trimeric gp120/gp41 Env protein bound thereto (FIG. 17).

In several embodiments, VLPs may be produced by transient transfection of cells with plasmids encoding Env. For example, 293T cells can be transfected with plasmids pNL4-3.Luc.R-E-, and a pCAGGS-based Env-expressing plasmid using polyethyleneimine as a transfection reagent. Two days later, supernatants are collected. Cell debris is then cleared by low speed centrifugation, filtration through a 0.45 μM filter and then pelleting particles at 50,000×g for 1 h, followed by a second spin in microfuge tubes at 25,000×g to remove residual culture medium. Virus-like particles are then resuspended in phosphate buffered saline at 1,000× the original concentration in the supernatant. Digests are performed by pelleting samples of virus and resuspending in optimal buffer for enzymes (e.g. endo H or chymotrypsin), adding various quantities of the enzyme(s) for a specified time at optimal temperature. After completion, particles are pelleted once again and resuspended in PBS. The resulting particles are expected to be substantially, if not completely, depleted of gp160 and other aberrant Env, while gp120/gp41 trimers remain intact. Alternative production methods include but are not limited to alternative plasmids, cell lines, infected cells, virus transduction. Alternative purification methods include, but are not limited to tangential flow filtration, sucrose density gradients, iodixanol gradients and others.

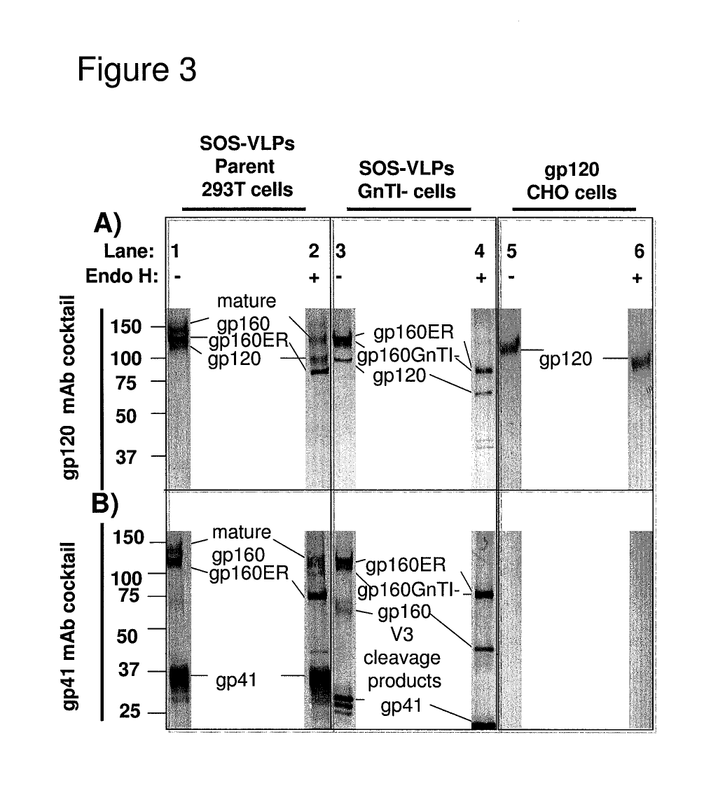

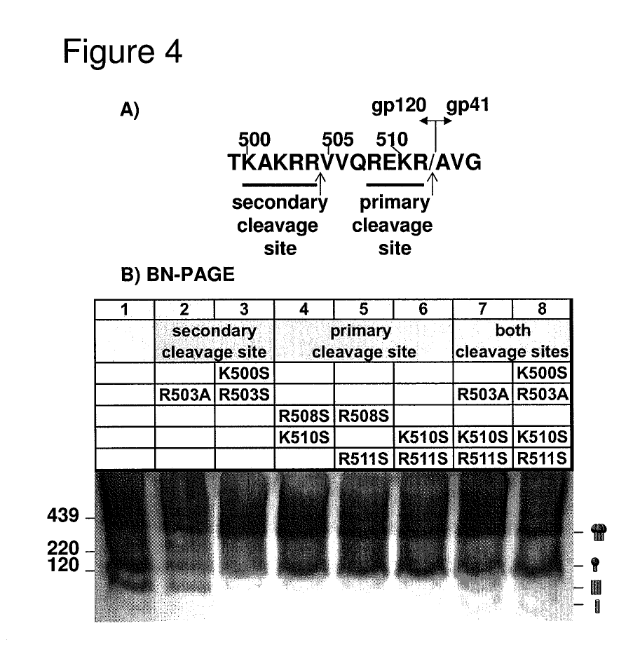

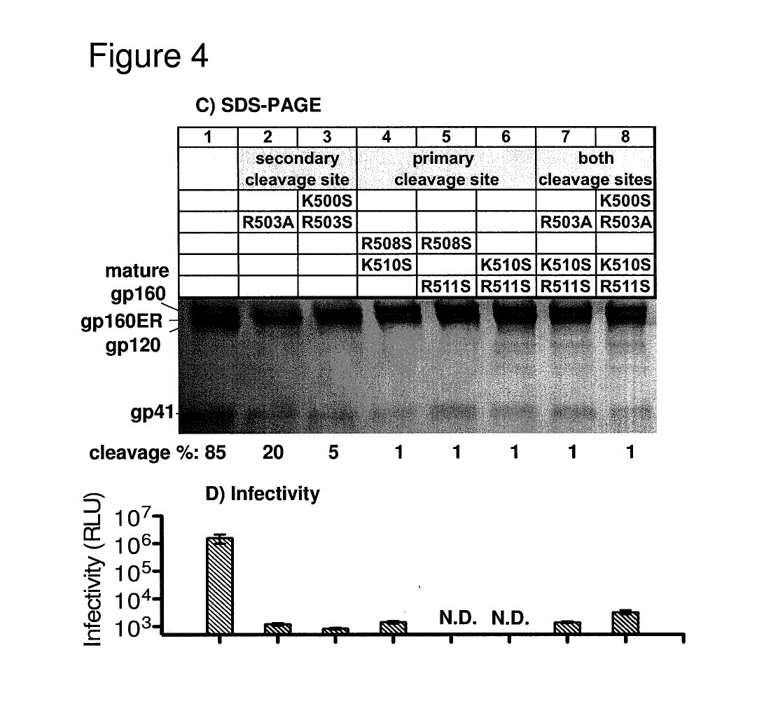

In various embodiments, Env may be produced in cell lines that are incapable of converting complex glycans into high mannose glycans, e.g. glucosylaminotransferase I (GnTI)-deficient 293S cells. Thus, in this case, all glycans are similar, but gp120/gp41 trimers differ from uncleaved gp160 in being proteolytically processed after the arginine or lysine at residue 511 of gp120 (LAI numbering) and possibly also the arginine at residue 504 (FIG. 4) and are substantially uniformly trimeric. Uncleaved gp160s are not processed after residue 504 or 511 of gp120 and may be monomeric, dimeric, trimeric, tetrameric or other high order complexes. FIG. 4 shows the effects of cleavage site mutations that result in VLPs largely bearing uncleaved gp160 and little or no native trimer. FIG. 4A shows the primary sequence of the JR-FL gp120-gp41 cleavage site. Residues are numbered according to the HXB2 sequence. Arrows indicate putative cleavage sites at residues 504 and 511. FIG. 4B shows an analysis of cleavage mutants in the JR-FL WT background (indicated by the table) by BN-PAGE-Western blot. FIG. 4C, shows an analysis of the same mutants by SDS-PAGE-Western blot analysis of WT-VLP cleavage site mutants. These blots were probed with both anti-gp120 and gp41 cocktails. Separate blots were performed with either cocktail alone to facilitate band identification (not shown; refer to FIG. 3). Cartoons indicate gp120/gp41 trimers and monomers and gp41 stumps. FIG. 4D shows the infectivity of cleavage site mutants (N.D.=not done). The data show that all cleavage site mutants have a dramatic effect on gp160 processing, leading to non-infectious VLPs that bear almost exclusively uncleaved gp160. Furthermore, uncleaved gp160 is primarily monomeric.

In several embodiments, one of the enzymes is a glycosidase (e.g. exoglycosidase or endoglycosidase). Preferably, the glycosidase is an endoglycosidase. More preferably, the endoglycosidase is endo H. In several embodiments, the glycosidase is any of endo H, endo F1, endo F2, endo F3, endo D, PNGase F, neuraminidases (also known as sialidase; including types alpha 1, 2, 3, 6, 8 and 9 varieties), mannosidases (including types alpha 1, 2, 3, 4, and 6 varieties), fucosidases (including alpha 1, 2, 3, 4, 6 varieties), O-glycanase (acetylgalactosaminidase), galactosidases (including endo beta 1, 4, alpha 1, 3, 6 and beta 1, 3, 4, 6 and other varieties), acetylglucosaminidase or combinations thereof.

In several embodiments, the methods of producing an immunogenic VLP involve using a concentration (represented by activity units) of 0.01 to about 10,000 units of endo H to digest about 10 ng Env equivalent of virus-like particles. In a particular embodiment, 500 units of endo H is used to digest 10 ng Env equivalent of VLPs. Other embodiments relate to methods wherein about 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 units of endo H are used to digest 10 ng Env equivalent of VLPs.

The glycosidase (e.g. endo H) may be incubated for a range of times in the methods of the present embodiments. For example, any of the aforementioned concentrations of endo H may be incubated with VLPs from about 0.1 to about 100 hours. In one embodiment, endo H treatment is conducted for about 24 hours and may go as long as 72 hours.

The temperature range for incubation with glycosidase (e.g. endo H) will be within the permissible range provided by the particular glycosidase being employed as understood by one of ordinary skill in the art. Similarly, digestions will be conducted in buffers with pH and chemical composition known to be optimal for said enzyme activity.

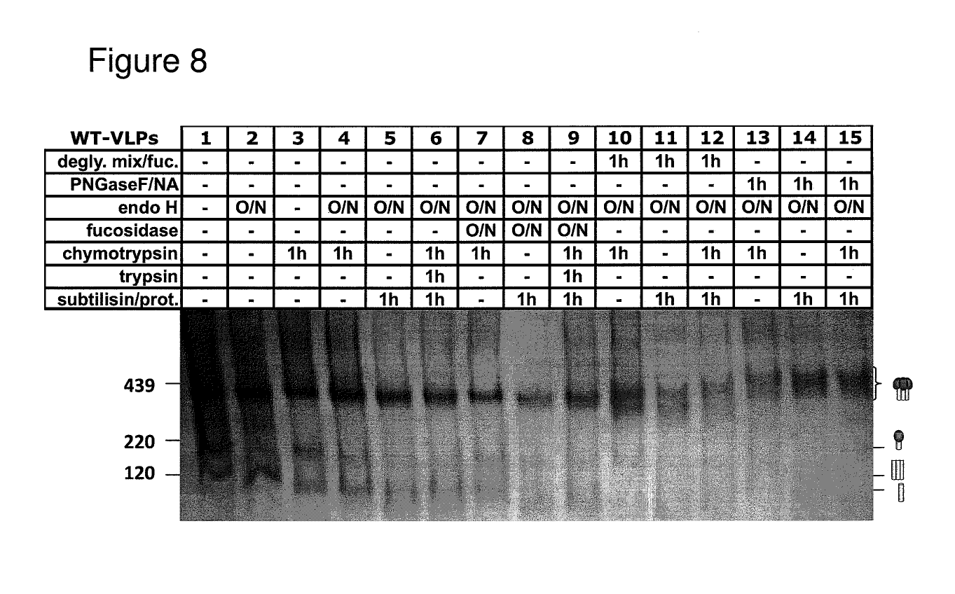

In several embodiments, one of the enzymes is a protease. In some embodiments, protease alone can be used without glycosidase priming. The protease can be any of those known in the art, for example, chymotrypsin, trypsin, pepsin, elastase, papain, subtilisin, proteinase K, endoproteinase Asp-N, endoproteinase Arg-C, endoproteinase Glu-C, endoproteinase Lys-C, thermolysin, clostripain, cathepsin C, pyroglutamate aminopeptidase, carboxypeptidase A, carboxypeptidase B, plasmin, and bromelain, or any combination thereof. A new method uses a combination of trypsin, chymotrypsin, proteinase K and subtilisin (FIGS. 8, 9, 11, 12, 16, and 20).

In several embodiments, the methods of producing an immunogenic VLP involve using a concentration (represented by activity units) of 0.01 to about 1,000 units of protease (e.g. chymotrypsin) to digest about 10 ng Env equivalents of virus-like particles. In a particular embodiment, 0.1 units of chymotrypsin is used to digest 10 ng Env equivalent of VLPs. Other embodiments relate to methods wherein about 100, 250, 500, 1,000, 2,000, 3000, 4,000, 5,000, 7,500, or 10,000 units of endo H are used to digest 10 ng Env equivalent of VLPs.

The temperature range for incubation with protease (e.g. chymotrypsin) will be within the permissible range provided by the particular protease being employed as understood by one of ordinary skill in the art.

In some embodiments, the methods of producing an immunogenic VLP involve digesting the VLP with one or more protease enzyme(s) without glycosidase digestion. The protease(s) may be chymotrypsin, trypsin, pepsin, elastase, papain, subtilisin, cathepsin C, pyroglutamate aminopeptidase, plasmin, and bromelain. About 0.01 to about 1,000 units of chymotrypsin or other protease(s) are contacted per about 10 ng Env equivalent of the plurality of virus-like particles. For example, in one embodiment, chymotrypsin is contacted with said virus-like particles for about 0.1 to 100 hours.

In several embodiments, the methods of producing an immunogenic VLP involve digesting the VLP first with glycosidase (e.g. endo H), then with protease (e.g. chymotrypsin). Not to be bound by theory, the high mannose (e.g. Man5-9GlcNac2) in the receptor binding site of uncleaved gp160ER exposes the underlying b12 binding patch (FIG. 2), which contributes to endo H-sensitivity, which in turn primes protease sensitivity by, for example, chymotrypsin (FIG. 17).

In other embodiments, the methods of producing an immunogenic VLP involve digesting the VLP first with a protease (e.g. chymotrypsin), then with a glycosidase (e.g. endo H). In further embodiments, one or more proteases alone or cocktails of proteases without the use of glycosidases can be used for producing an immunogenic VLP.

Several embodiments of the present invention relate to conditions of enzymatic digestion that maximize elimination of gp160ER from VLP while leaving gp120/gp41 trimers unaffected. In one aspect, a method of completely removing gp160ER by enzymatic digestion is provided. Similar methods can selectively eliminate mature gp160 and gp41 stumps.

1. Analytical Framework.

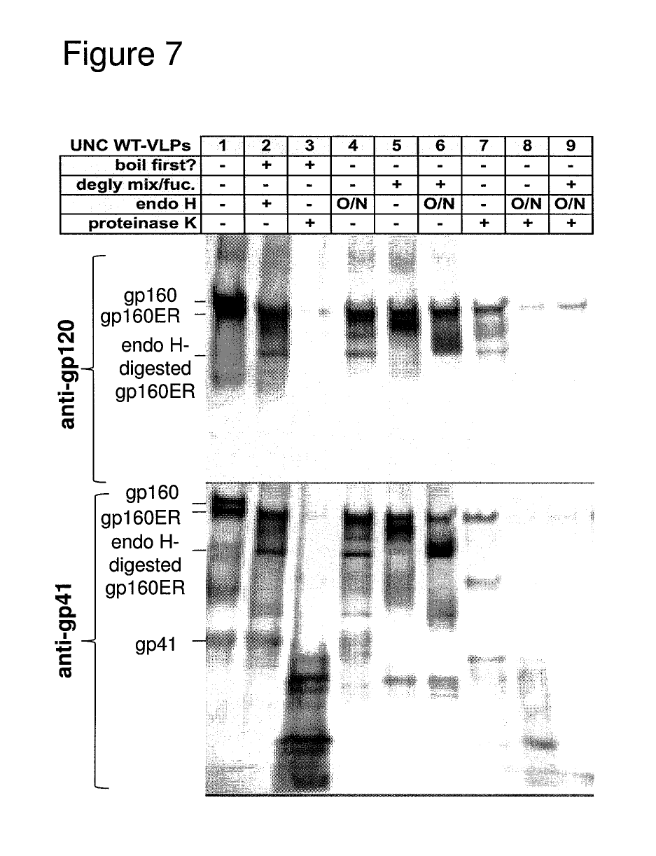

i) The removal of gp160 (both mature and ER varieties) can be tracked by BN-PAGE and SDS-PAGE-Western blots, as in FIGS. 7, 8 & 9.

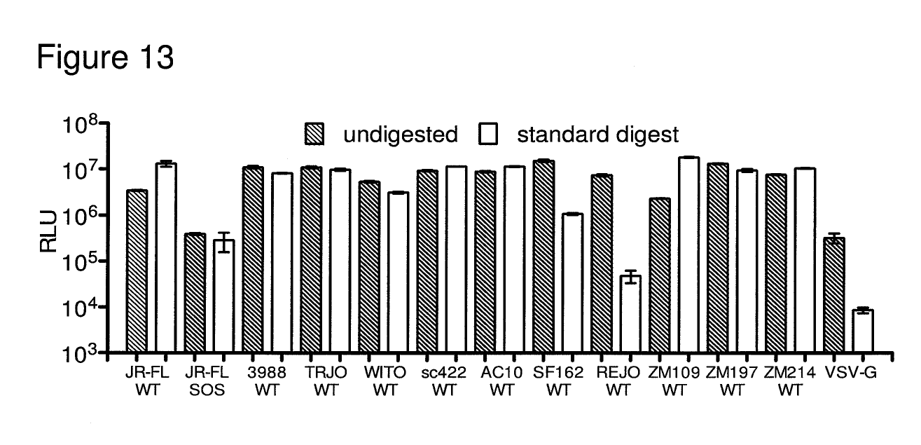

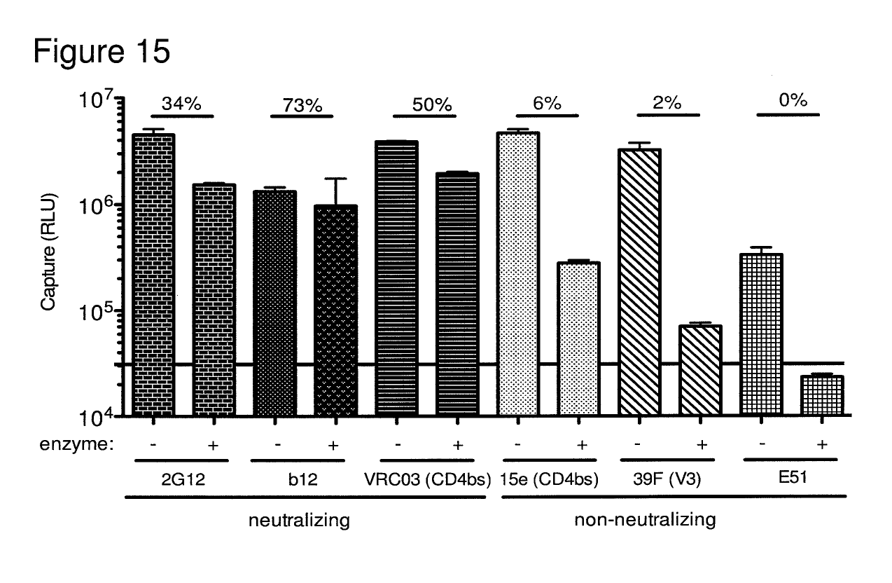

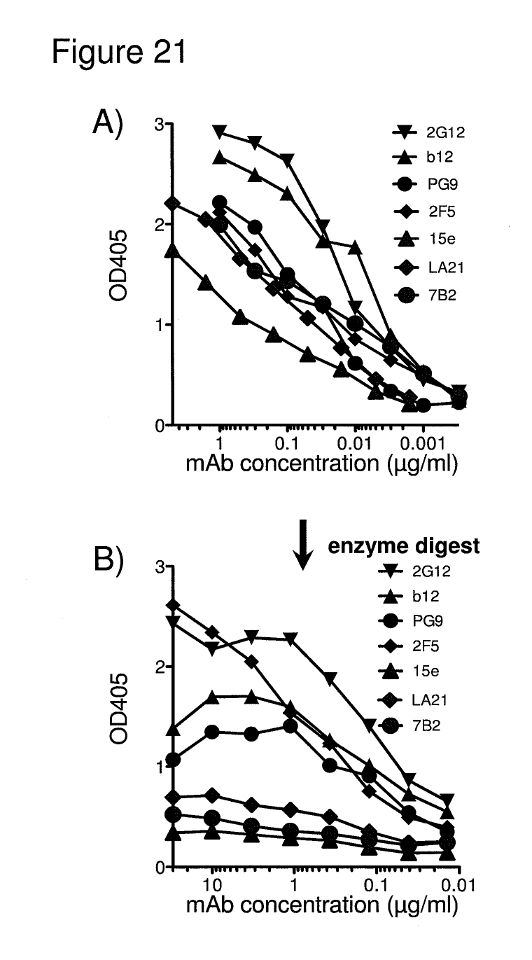

ii) The selective loss of virus capture by non-neutralizing mAbs can be monitored as in FIG. 15. Prolonged digests, if needed, may lead to a drop in infectivity, obfuscating the measurement of capture by an infectious readout. In this case, capture is measured by p24 ELISA (51, 70, 76). A loss in infectivity is usually not a problem, however (FIG. 13). In FIG. 13, various Env-VLPs were subjected to mock or enzyme treatment (in this case an overnight endo H digest followed by digestion with a mixture of chymotrypsin, trypsin, subtilisin and proteinase K), washed with PBS and then assessed for their ability to infect CF2.CD4.CCR5 cells. RLU=relative light units. An alternative method to track the depletion of non-functional Env is by VLP ELISA. In FIG. 21, effective digests of the E168K+N189A mutant of JR-FL WT VLPs (using a cocktail trypsin, chymotrypsin, proteinase K and subtilisin in an overnight digest in thin walled PCR tubes) led to a scenario where neutralizing monoclonal antibodies (2G12, b12, PG9 and 2F5) bind, but non-neutralizing monoclonal antibodies (15e, LA21 and 7B2) do not (FIG. 21B). This contrasts to the epitope exposure before digestion, where diverse monoclonal antibodies can bind particles, regardless of any neutralizing activity (FIG. 21A).

iii) Avidity can lead to artifacts in virus capture (51, 76). In-solution mAb-virus binding followed by capture is, however, more stringent (51, 65, 76). Effective self- and cross-competition of unlabeled and biotinylated mAbs in solution can further confirm capture specificity (51). For example, if b12 capture can be partially competed off by CD4bs mAb 15e, this would indicate 15e binding to residual gp160—a sign of incomplete digestion.

iv) Tracking gp160 removal by virus capture could suffer from interference by the gp120/gp41 trimer. While non-neutralizing mAbs should not capture via trimer, the abovementioned avidity effects may come into play (76). It might be useful, therefore, to assess gp160 digestion on particles that bear only this aberrant species of Env. In this case, because all (non-functional) Env species should be digested, it might be easier to gauge progress by virus capture. A virus that bears only gp160ER is possible by producing particles without Env plasmids, termed “bald VLPs” and then mixing these with the supernatant from Env-expressing cells. Here, Env-expressing cells that die leave copious gp160ER in the supernatant. This can bind to the membranes of bald VLPs via the hydrophobic gp41 moiety. Similarly, mature gp160 removal can be gauged by expressing substrate VLPs that bear Envs mutated to prevent gp160 cleavage by furin (i.e. an UNC mutant, FIGS. 4, 7 and 18). An alternative approach is to use VLPs of the JR-FL mutant A328G of gp120. This mutant is known to exhibit only pure trimer after digests, so represents a useful positive control for gp160 removal (FIG. 11, lanes 13 and 14). Following digestion of these substrate VLPs, mixing with monoclonal antibodies and capture by an immobilized anti-Fc antibody, the readout of virus capture in this case would be p24, as above. This approach should also increase the clarity by which the loss of gp160ER in SDS-PAGE and BN-PAGE is monitored. In each case, conditions that deplete all non-functional forms of Env are determined. Once appropriate digest conditions are identified, they could then be checked to ensure that parent gp120/gp41 trimers are not affected. An alternative to mutant A328G VLPs would be GnTI-VLPs whose gp120/gp41 trimers are endo H-sensitive (FIG. 11, lanes 3 and 4) (6).

v) The products of digestion may not dissociate and could even bind back non-specifically to VLPs (51). As needed, additional washes are performed. Reducing agent can be included in washes to help dissociate material attached by covalent bonds (see also below). Low concentrations of chaotrope or detergent will also be evaluated. Progress can be monitored by SDS-PAGE (fragments should disappear) and by virus capture.

vi) The retention of native gp120/gp41 trimer architecture can be assessed by BN-PAGE shifts as in FIG. 16, by infectivity and neutralization assays with mAbs, where possible. VLP ELISA, as in FIG. 21, can be used to further assess the retention of native trimers.

vii) The removal of all forms of uncleaved gp160 can be assessed sparingly by staining VLPs with gold-labeled mAbs and electron microscopy. Previously, it was found that non-neutralizing mAb b6, and neutralizing mAb b12 could each stain SOS-VLPs specifically, highlighting the presence of non-native Env (see FIG. 12 in ref (65)). Successful digests should eliminate b6 binding, but retain b12 binding.

### 2. VLP Digestion by Endo H/Chymotrypsin.

i) Endo H Digestion:

Approximately 1 μl of 1,000×SOS-VLPs (˜10 ng Env equivalents) is digested with 500 U endo H overnight, followed by a chymotrypsin digest (100 U) for 1 h all at 37° C. in optimal buffer. The observation that glycan-depleted gp160ER is removed by subsequent protease treatment, but the gp120/gp41 trimers remained, suggests that endo H primes chymotrypsin digestion (FIGS. 7 and 8, compare lanes 2 and 4). Thus, endo H appears to be the rate-limiting step. Longer digests and larger amounts of enzyme deplete gp160ER more effectively, suggesting that improvements are possible (not shown; (61, 81)). Overnight digests using 15,000, 5,000, 1,500 and 500 U of endo H and a mock sample may be compared. Then, using optimal enzyme units and a mock, digestion times of 1 h, 24 h, 48 h & 72 h can be compared. Success can be gauged by SDS-PAGE-Western blot: the gp160ER band will be observed to fully drop in size to the glycan-depleted form in FIG. 7, lane 4. These altered protocols should not affect the gp120/gp41 trimer.

Embodiments of the present invention include a range of endo H concentration, for example, from about 0.01 units of endo H to about 100,000 units of endo H per about 10 ng Env equivalents of VLPs (i.e. the quantity of VLPs representing that exhibit 10 ng of Env), and any particular number or range of units of endo H in between. Also, embodiments of the present invention include a range of digestion times with any concentration of endo H, for example, from about 0.1 hours to about 100 hours, and any particular duration or range of time in between.

ii) Chymotrypsin Digestion:

Embodiments of the present invention include a range of chymotrypsin concentration, for example, from about 0.01 units of chymotrypsin to about 1,000 units of chymotrypsin per about 10 ng Env equivalents of virus-like particles (i.e. the quantity of virus-like particles representing 10 ng of Env), and any particular number or range of units of chymotrypsin in between. Also, embodiments of the present invention include a range of digestion time with any concentration of chymotrypsin, for example, from about 0.1 hours to about 100 hours, and any particular duration or range of time in between.

Gp160ER mature gp160 and gp41 stump removal/trimer integrity can be monitored, for example, by BN-PAGE and SDS-PAGE-Westerns, as in FIGS. 7 & 8.

3. Additional Approaches to Remove gp160ER, Mature gp160 and gp41 Stumps.

Several embodiments provide for uncleaved gp160 removal involving modulation of the conformational flexibility, a subspecies of multimers that may contribute to resistance of degradation by glycosidase and protease (e.g. endo H and chymotrypsin), or the presence of complex glycans, as in mature gp160, that might require alternative glycosidases and modified digestion conditions to remove these glycans (FIG. 2). For example, various embodiments relate to the following treatments to enhance or facilitate degradation by glycosidase and/or protease (e.g. endo H and chymotrypsin):

i) Conformational Fixation.

Reports suggest that Env can sample different conformations (29, 48, 62, 73, 104, 106). Rigidifying uncleaved gp160 or gp41 stumps might make it an easier target for digestion. This could be achieved by:

a) Complexing it with non-neutralizing mAbs. EM data suggests that mAb binding can cause structural changes in Env (58). These changes might enhance digestion sensitivity. Non-neutralizing mAbs can be tested, prioritizing V3 loop and CD4i mAbs that do not obscure the outer domain targeted for digestion (FIG. 1A). If MAbs are not fully digested and removed, as needed, they will be eluted from VLPs by exposure to glycine pH2.2. The integrity of gp120/gp41 Env trimers may be checked in BN-PAGE shifts, as in FIG. 16. VLP ELISA, as in FIG. 21, may also be used to examine trimer integrity.

b) Paraformaldehyde crosslinking. At low concentrations of crosslinker, conformational epitopes on gp120/gp41 trimers have been reported to be preserved (84). Trimer preservation can be monitored by BN-PAGE shifts, as in FIG. 16 and by VLP ELISA, as in FIG. 21.

c) A panel of JR-FL cysteine substitution mutants to stabilize gp120/gp41 trimers laterally, using double cysteine mutants to introduce V-loop-spanning disulfides. These new disulfides might affect the gp120/gp41 trimer, and/or uncleaved gp160 and may affect their rigidity and thus sensitivity to digestion.

d) H66A or W69L or other mutants that limit spontaneous sampling of the CD4-bound conformation and therefore may limit Env flexibility (as detailed in refs (44, 45)).

ii) Gp160 Forms a Resistant Subspecies of Multimers.

Only gp160 monomers in BN-PAGE are observed, but various embodiments provide for a subspecies of labile multimers (26, 27). Alternatively, gp160 may interact with other membrane components, with a concomitant gain in enzyme-resistance. In a variety of embodiments, SOS-VLPs are treated with the crosslinker BS3 (65). The components of any cross-linked bands can be identified by probing duplicate SDS-PAGE Western blots with anti-gp41 and anti-gp120 mAb cocktails and by checking endo H susceptibility, as in FIGS. 3 and 7. Covalent multimers can be observed by running gels with and without reducing agents. If multimers are found, various approaches could dissociate them according to embodiments of the present invention. In one embodiment, non-covalent multimers may be sensitive to and treated with non-ionic detergents at concentrations below that sufficient to lyse VLPs. In one aspect, enzyme digestions are performed in the presence of detergent and then VLPs are re-isolated by centrifugation. The observation that solubilized VLPs are more efficiently digested (FIG. 14) supports this approach. Another embodiment provides a method for disrupting multimers by SDS-PAGE Westerns with and without BS3, as above, and identifying a drop in BS3-crosslinked multimer. In one embodiment, if gp160ER associates with another membrane component, VLPs are expressed in cells in which the protein is absent. Yet another embodiment involves brief exposure to a chaotrope to break apart labile multimers. Covalent multimers may be sensitive to low concentrations of reducing agents (105). Previously, gp160 precursors were found to have inter-gp120 V3 loop or gp41 disulfides at the immunodominant loop, providing a precedent (5, 71, 105).

Several embodiments are drawn to balancing conditions that affect uncleaved gp160 but not gp120/gp41 trimers. In various embodiments, authentic trimers remain intact after exposure to low concentrations of non-ionic detergent, chaotropes, reducing agents and non-neutralizing monoclonal or polyclonal antibodies. Digestive enzymes are also unaffected by low concentrations of non-ionic detergents. In a variety of embodiments, controlled digestions of the gp120 monomer are performed in the presence of chaotropes and/or reducing agents.

4. Improving the Efficiency of gp160ER Removal.

i) Alternative Glycosidases.

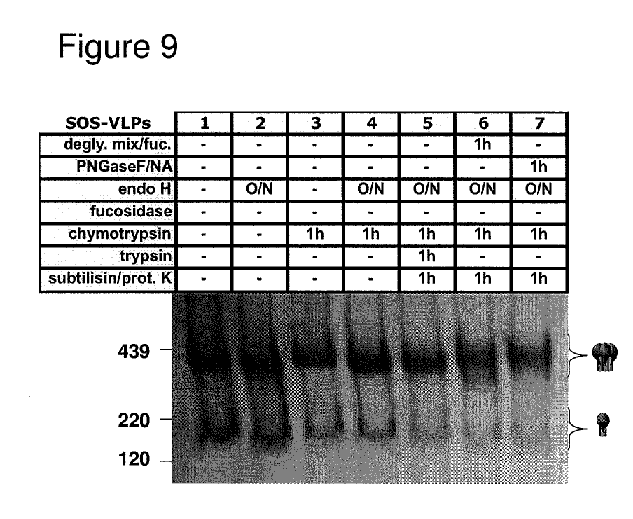

In several embodiments, the glycosidase is endo H, because it selectively removes oligomannose glycans near their base, priming gp160ER for protease digestion (FIG. 1 in (6)). The slow kinetics of endo H could be due to the difficulty of gp160ER as a target and/or the limitations of endo H as a compatible enzyme. To remove glycans from mature gp160, either the densely-packed, high mannose glycans of the silent domain or the complex glycans of the neutralizing face could be targeted (FIG. 2). Thus, other endoglycosidases provided in several embodiments might be more effective. In another embodiment, the glycosidase is Endo F1, which has a similar specificity to endo H. In some embodiments, the glycosidase is PNGase F, which completely removes glycans at their base, leading to the conversion of a relatively hydrophobic asparagine-linked glycan into a hydrophilic aspartic acid (24). In other embodiments, the glycosidase is an exoglycosidase such as neuraminidase (also known as sialidase; including types alpha 1, 2, 3, 6, 8 and 9 varieties) and mannosidase (including types alpha 1, 2, 3, 4, and 6 varieties) that affect glycan termini. These enzymes can digest gp120/gp41 trimers, and moderately increase or decrease infectivity, respectively, without appreciably affect neutralization sensitivity (61). Although they may not be useful alone, they may prime for endo H in sequential digests. Other glycosidases may affect complex, high mannose and O-linked glycans include endo F2, endo F3, endo D, neuraminidases, fucosidases (including alpha 1, 2, 3, 4, 6 varieties), O-glycanase (acetylgalactosaminidase), galactosidases (including endo beta 1, 4, alpha 1, 3, 6 and beta 1, 3, 4, 6 and other varieties), acetylglucosaminidase or combinations thereof. FIG. 9 shows a BN-PAGE-Western blot analysis of glycosidase-protease digests of SOS-VLPs, where VLPs were treated with various combinations of glycosidases and proteases, as indicated. This analysis suggests that conditions involving endo H all deplete non-functional Env monomer. However, the trimer also appears affected by these digests. A cocktail of proteases was more effective than chymotrypsin alone (FIG. 9, compare lanes 4 and 5). Conditions using other glycosidases had marked effects on the trimer (FIG. 9, lanes 6 and 7). An overnight endo H digest followed by a cocktail of proteases was the most effective treatment shown in FIG. 9.

It will be understood that any of the aforementioned glycosidases may be used alone or in combination, wherein the concentration of each glycosidase includes a range, for example, from about 0.01 units of glycosidase to about 10,000 units of glycosidase per about 10 ng Env equivalents of virus-like particles (i.e. the quantity of virus-like particles representing 10 ng of Env), and any particular number or range of units of glycosidase in between. Also, embodiments of the present invention include a range of digestion times with any concentration of glycosidase, for example, from about 0.1 hours to about 100 hours, and any particular duration or range of time in between.

ii) Alternative Proteases.

Various embodiments relate to screening proteases for optimal digestion of gp160ER. For example, such proteases include but are not limited to chymotrypsin, trypsin, pepsin, elastase, papain, subtilisin, proteinase K, endoproteinase Asp-N, endoproteinase Arg-C, endoproteinase Glu-C, endoproteinase Lys-C, thermolysin, clostripain cathepsin C, pyroglutamate aminopeptidase, carboxypeptidase A, carboxypeptidase B, plasmin, and bromelain, or any combination thereof. Preferably, the protease is chymotrypsin. A combination of trypsin, chymotrypsin, proteinase K and subtilisin (identified in FIG. 9, lane 5) are highly effective in digests using thin walled tubes overnight at 37° C., without the need for endo H priming. The effectiveness of this approach, particularly using the WT E168K mutant is shown in FIGS. 16 and 20. FIG. 20 shows the effect of enzyme digests on the JR-FL E168K mutant, where almost all junk Env is removed. FIG. 16 also shows that E168K+N189A WT-VLPs exhibit near perfect pure trimers after digests that represent a slight improvement in purity compared to digested WT-VLPs (FIG. 16, compare lane 1 in parts B and C).

Embodiments of the present invention include a range of protease concentrations, for example, from about 0.01 units of protease to about 1,000 units of protease per about 10 ng Env equivalents of VLPs (i.e. the quantity of VLPs representing 10 ng of Env), and any particular number or range of units of protease in between. Also, embodiments of the present invention include a range of digestion times with any concentration of protease, for example, from about 0.1 hours to about 100 hours, and any particular duration or range of time in between.

iii) Combination Approaches.

Some digest products might provide targets for other enzymes. For example, exoproteases might augment “nicks” caused by endoproteases. Embodiments involve using any enzyme combining it in digests with other enzymes, either iteratively or simultaneously. To avoid possible effects of proteases on each another, iterative digests can be separated by washes. Additional embodiments include varying the sequence of different digests. For example, trypsin is required for chymotrypsin maturation from its zymogen precursor. Thus, if chymotrypsin is not fully mature, its combination with trypsin might boost its activity.

iv) Alternative VLP Substrates.

Our data indicate that the selective digestion and removal of uncleaved gp160 and gp41 stumps, leaving trimers behind, is a universally applicable method that is effective for various Env variants, mutants and clades (FIGS. 11, 12, and 18). Embodiments of the present invention include Envs from various clades (FIG. 12). In one embodiment, JR-FL SOS-VLP is selected as a VLP substrate, due to its high expression (65), functionality, and gp120/gp41 processing. The SOS mutation also eliminates gp120 shedding. It is possible, however, that the SOS mutation, the protein sequence, conformation or the producer cells render digestions particularly difficult. Therefore, other embodiments provide alternative VLP substrates. In one embodiment, JR-FL WT-VLPs are selected as substrate particles. In another embodiment, a full-length molecular clone, pLAI-JR-FL, is selected to express VLPs, as in ref (51). These bear substantially less non-functional Env that may be easier to remove. Inactivation by AT-2 can resolve any safety issues. In another embodiment, particles are produced in GnTI-(N-acetylglucosamine transferase I)-deficient 293S cells. These express uncleaved gp160 exclusively of the gp160ER variety, allowing the facile removal of all aberrant Env by endo H/chymotrypsin digestion. Due to the conformational differences between gp120/gp41 trimers and gp160ER, the former survive the enzyme treatments. In another embodiment, a “globally” resistant mutant, D674A, is selected, which reduces neutralization by b12, 2F5 and broadly neutralizing HIV+ plasmas. This will allow us to use harsher digests without affecting the gp120/gp41 trimer. Different Env clones and mutants may affect gp160ER conformation, sequence and glycosylation, contributing to digest efficiency. Yet another embodiment includes use of producer cell lines that may influence glycosylation patterns, affecting digestion. Alternative cell lines would include, but are not limited to GnTI-, COS, CHO, BSC40, and HeLa. The different proteomic landscape of the plasma membrane might also enhance digestion. As shown in FIGS. 16, 20, and 21, JR-FL WT E168K and WT E168K+N189A mutants exhibit a lower ratio of junk Env to trimer, which has, together with an enhanced digestion approach (overnight digestion with chymotrypsin, trypsin, subtilisin and proteinase K in thin walled PCR tubes) resulted in pure “trimer VLPs”.

v) Scaling Back Digests:

In several embodiments, methods relate to using digestion conditions (e.g. concentrations, duration of treatment, etc.) that place a greater emphasis on completely eliminating uncleaved gp160 over the fate of gp120/gp41 trimers, which may be intact or partially digested. For example, papain-treated gp120/gp41 trimers, although affected in their mobility and separation properties in BN-PAGE, still selectively bind only nAbs. Similarly, mannosidase-treated gp120/gp41 trimers, although not infectious, retain the resistant features of the gp120/gp41 trimer. Proteinase K and subtilisin ablate uncleaved gp160, but do not completely digest the gp120/gp41 trimer (FIG. 8, lane 6). Therefore, in several embodiments, digests are scaled back to find conditions where the uncleaved gp160 is still digested, but the gp120/gp41 trimer is unaffected. For example, lower concentrations and briefer incubations may be used. If conditions are still too harsh, digests can be retested using the more resistant D674A mutant.

vi) Other Forms of Non-Functional Env:

Mature gp160 is another form of Env that can contaminate VLPs (11, 19). However, given its ‘open’ conformation, appropriate conditions for digests are contemplated herein (see UNC mutant in FIG. 7). Gp41 stumps are also not problematic. As shown in FIG. 20, several embodiments including overnight digestion with chymotrypsin, trypsin, subtilisin and proteinase K in thin walled PCR tubes removed non-functional Env from JR-FL WT E168K VLPs. The ELISA in FIG. 21 reflects the purity of the remaining trimers. Here, after digestion, only neutralizing epitopes are exposed (FIG. 21B), in contrast to the undigested VLPs, that also expose non-neutralizing epitopes (FIG. 21A).

B. Soluble Forms of Pure Authentic Env gp120/gp41 Trimers.

Several embodiments of the present invention are drawn to pure soluble Env gp120/gp41 trimers. In general, and without being bound by theory, detergent-solubilized Env is more sensitive to enzymes compared to their membrane-presented equivalents on VLP surfaces. In FIG. 14, VLPs were solubilized in 1% triton and then subjected to 15 min endo H and 1 h protease treatments at 37° C. Solubilized particles digested with endo H followed by chymotrypsin yielded completely pure trimers (FIG. 14, lane 4). Several embodiments provide an immunogenic composition comprising immunodeficiency virus soluble trimeric gp120/gp41 Env protein and substantially lacking soluble gp160 Env protein and/or soluble gp41 and/or gp120, and methods of making the same.

In one embodiment, a method for producing soluble Env gp120/gp41 trimer involves providing a producer cell which expresses immunodeficiency virus trimeric gp120/gp41 Env protein and uncleaved Env protein on the surface; contacting the producer cell with a concentration of an extraction agent (e.g. detergent) effective to release soluble gp120/gp41 trimeric and gp160 Env protein from the surface of the producer cell; and contacting the soluble gp120/gp41 trimeric and gp160 Env protein preferably with enzyme(s) that substantially and selectively remove the soluble gp160 Env protein to generate a composition comprising substantially only soluble trimeric gp120/gp41 Env protein (FIG. 14). FIG. 19 graphically depicts the purifying effects of glycosidase/protease digests from detergent-lysed particles, Env-transfected cells, infected cells and other tissue sources where authentic trimers are presented on membranes. Detergent lysis results in a mixture of soluble Env species. After enzyme digestion, however, only soluble gp120/gp41 Env trimers closely resembling the equivalent authentic gp120/gp41 Env trimers on membranes remain.

The producer cell can be any cellular source of Env expression known in the art. For example, in one embodiment the producer cell is an immunodeficiency virus-like particle expressing wild-type Env protein, such as the VLPs and/or cell lines described above. In another embodiment, the producer cell is an immunodeficiency virus-like particle-expressing mutant Env protein having a disulfide linkage between gp120 and gp41. In yet another embodiment, the producer cell is a transfected cell ectopically expressing wild-type or mutant Env protein. In another embodiment, the producer cell is a virally transduced cell ectopically expressing wild-type or mutant Env protein. In another embodiment, the producer cell is an immunodeficiency virus-infected cell. It will be understood that the Env protein in any of the embodiments of the present invention include wild-type, mutant, or genetic variants from different clades (FIG. 11, 12, 18).

The foregoing embodiments relate to methods of producing soluble Env gp120/gp41 trimer immunogen in which Env proteins are extracted from membranes (e.g. by detergent) prior to treatment with enzyme (e.g. glycosidase and protease). In other embodiments, the producer cell is first treated with enzyme (e.g. glycosidase and protease) to digest and substantially remove gp160 while leaving thegp120/gp41 Env trimer intact, followed by extraction of the gp120/gp41 Env trimer (e.g. by detergent) (as in FIG. 19). The aforementioned glycosidases and proteases, their concentrations, incubation times, and temperature ranges are applicable to embodiments pertaining to soluble gp120/gp41 Env trimer immunogen and methods of making the same.

The Env proteins can be extracted from the producer cell with any agent known in the art useful for removing proteins from cell membranes. In one embodiment, detergent, such as non-ionic detergent (e.g. triton) is used. A range of concentrations of detergent is permissible. For example, the concentration of triton is about 0.01% to about 1%, preferably about 0.15%.

Various embodiments relate to conditions for increasing stability, production and purification of soluble gp120/gp41 Env trimers.

1. Stability

In contrast to the labile soluble SOS gp140 trimers (88), solubilized SOS-VLP trimers (in 0.15% triton) survive incubation for over 1 h with enzymes at 37° C., followed by BN-PAGE for 3 h at 4° C. (FIG. 14, lane 4). Thus, its original membrane context and TM domain may lead to enhanced stability compared to Env forms that are expressed in a naturally soluble form. Stability of solubilized trimers in various scenarios can be assessed using BN-PAGE-Western blots as a readout:

i) Purification. Soluble trimer stability can be checked following the rigors of purification, time, temperatures, freeze-thaws and exposure to various buffers, including high salt, low pH, high pH and chaotropes.

ii) Exposure to animal sera. Co-incubations of soluble trimers with animal sera at 37° C. does not affect VLP gp120/gp41 trimers (not shown).

iii) Co-formulation with adjuvants. In various embodiments, soluble gp120/gp41 Env trimer is co-formulated with adjuvants, for example, Ribi, QS21, Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B (CTB; and its derivative), ISCOMS, saponins, TDM, CWS emulsion, poly I:C, virosomes, alum, alhydrogel, CD40L, BAFF, and APRIL, C3d. Each of these foster the production of Abs directed to conformational epitopes, implying that they are non-denaturing.

Various embodiments consider the following options to increase stability: i) the use of gp120/gp41 trimers containing carefully placed cysteine substitutions to increase lateral and apical stability, ii) formaldehyde treatment (84) and iii) the use of neutralization-resistant D674A mutant gp120/gp41 trimers.

2. Production and Purification

Generating sufficient quantities of soluble gp120/gp41 trimer may be challenging. Only limited amounts can be obtained from VLPs (˜10 μg Env equivalents/L). Therefore, in one embodiment, soluble gp120/gp41 Env trimer is isolated by detergent extraction of transfected cells. Nuclei will first be pelleted. In several embodiments, following enzyme treatments, gp120/gp41 trimers can be purified by chromatography. In one aspect, gp120/gp41 trimers are purified by lectin chromatography, for example, by Galanthus nivalis lectin chromatography. In another embodiment, soluble Env gp120/gp41 trimers are further purified by size exclusion chromatography. In another aspect, they are purified by ion exchange chromatography. A multi-step purification protocol can be performed in any sequence, and can be determined empirically e.g. if digestion of crude lysates is inefficient, glycoproteins can be first isolated by lectin, followed by digests. If digestions fail to remove all non-functional Env, efficacy might be improved by first purifying plasma membranes, using a kit (Qiagen Qproteome plasma membrane kit) or by floating them on iodixanol.

Several embodiments involve using different enzymes, detergents and purification steps (e.g. anion exchange). Various embodiments relate to different producer cell lines. Additionally, production and purification methods can include lactacystin, a proteasome inhibitor, which increases Env expression ˜3 fold (12). If antigen production is still problematic, immunizations with lower quantity of immunogen may feasible by using carbopol or AS01B adjuvants, which are both dose-sparing. Soluble trimer purity can be assessed by silver stain SDS-PAGE (>90% for immunizations) and removal of host cell membrane proteins can be determined by ELISA, as in (43). A soluble uncleaved gp140 (consisting of gp120 and gp41 ectodomain) of the matched isolate expressed by transient transfection can serve as a control for these procedures (4).

### C. Antigenicity and Optimization of Prospective Immunogens

Prospective immunogens can be checked by BN-PAGE, as in FIG. 14, FIG. 16 and FIG. 20. A comparison of solubilized and particulate gp120/gp41 trimers revealed almost identical topology (FIG. 11 in ref (65)); gp120-specific nAbs bind to both forms of trimer and non-neutralizing mAbs bind neither. However, gp41-specific MPER mAbs preferentially recognize soluble gp120/gp41 trimers (in FIG. 11 in ref (65)), compare Z13 binding in lanes 6 & 12). Thus, local changes may lift constraints on gp41 mAb binding, making solublegp120/gp41 trimers particularly attractive MPER immunogens.

Hyperimmunization.

An embodiment of the present invention involves administering multiple vaccine shots over an extended immunization regime (3, 50, 55, 92).

Complexing VLPs with Naked-VLP IgG.

Complexing SOS-VLPs with Naked-VLP IgG may augment Env responses. One embodiment involves raising IgG against digested Naked-VLPs, to match the IgG with immunogens. Doses of IgG will initially be per immunization 300 μl of 10 mg/ml. Control animals will receive masking IgG only. In a previous similar experiment, a similar dose of IgG was undetectable in sera taken 2 weeks later, presumably due to its distribution and decay.

Alternative Env Prototypes or Producer Cells.

Options for other Env prototypes include sensitive Envs from the panel of JR-FL gp120/gp41 trimer mutants and GnTI-gp120/gp41 trimers that mimic the parent gp120/gp41 trimer's resistance to non-neutralizing mAbs, but exhibit enhanced sensitivity to certain nAbs, due to a thinner glycan shell (6). Due to GnTI-gp120/gp41 trimers' sensitivity to endo H, digests will omit this enzyme. VLPs may be produced in alternative cell lines, e.g. CHO.

Sequential Immunization.

To provide an easy “on ramp” for nAb development, one embodiment involves evaluate priming with sensitive JR-FL mutant gp120/gp41 Env trimers and boosting with resistant ones. For example, if the sensitive gp120/gp41 trimers elicit both V3 loop and CD4bs NAbs, boosting with resistant gp120/gp41 trimers may favor the CD4bs NAbs.

Soluble Trimers.

Soluble trimers (as above) might favor MPER nAbs and eliminate the problems incurred of possible “anti-membrane” responses generated to VLPs.

Intradermal Immunization.

One embodiment pertains to the intradermal route of immunization, which may have advantages over the standard intramuscular route, as described in ref (22).

Alternative Purification Methods.

VLP pelleting may affect their immunogenic properties. One embodiment involves using tangential flow filtration as an alternative way to purify VLPs. Other embodiments involve using iodixanol or sucrose gradients (57).

Co-Expression of Immunomodulators.

One embodiment relates to VLPs bearing VSV-G in addition to Env. VSV-G is amphotropic, allowing fusion with rabbit cells, facilitating antigen presentation and possibly augmenting B cell responses (40, 47, 60, 78). Similarly, in other embodiments, CD40L is used as an immunomodulator (90).

### D. Approaches to Broaden nAb Responses

Several embodiments relate to strategies for broadening nAb responses (reviewed in (95)):

i) Polyvalent Mixtures of VLP-Env gp120/gp41 Trimers.

Compositions and methods of the present invention can apply to various Envs and mutants (FIG. 11, 12). Clade C Envs have features that may be worth investigating in a vaccine format (52, 54, 79, 91). Polyvalent approaches might foster responses to shared epitopes (17, 39, 97, 102). In one embodiment, Env prototypes from clades A, B and C are selected and 1:1:1 mixture containing a 1/3 dose of each. Further embodiments include Envs from various other clades, e.g. D, CRF01_AE, F, and G. Envs can be selected based on efficient expression and neutralization-resistance (FIG. 1 of (19)).

ii) VLPs Bearing Mixtures of Different Envs.

Several embodiments relate to VLPs bearing mixtures of Envs by cotransfecting various Env plasmids (e.g. equal amounts of clade A, B and C Envs). Resulting VLPs may bear Envs from all 3 clades and possibly gp120/gp41 trimers comprising of mixed Env protomers.

iii) Sequential Immunization with Different Env-VLPs.

The possible problem of immunodominance in polyvalent immunizations may be overcome by sequential immunization. Thus, if initial immunization with one Env-VLP primes type-specific and broad nAbs, a boost with a distinct Env-VLP might expand responses to the conserved epitopes.

iv) Env VLPs Constructed from Centralized Env Genes.

In one embodiment, centralized Envs are used, which might minimize the antigenic distance between the immunogen and the range of isolates to elicit neutralization against (32, 33).

v) Alternative Adjuvants.

Several embodiments are drawn to a variety of adjuvants, as long as they have no adverse effects on VLPs or gp120/gp41 trimers. Possibilities include QS21 and CpG (Coley), Carbopol, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, C3d and APRIL.

E. Selection of Neutralizing Antibodies from B Cell Repertoires Using Particles Bearing Authentic gp120/gp41 Env Trimers or Soluble Authentic gp120/gp41 Env Trimers

The particles (e.g. VLPs or microparticles) having gp120/gp41 Env trimers or soluble gp120/gp41 trimers described herein may be used to select B cells expressing neutralizing antibodies. For example, particles could be produced by transfection of 293T cells that were previously cell-surface biotinylated. Particles can then be treated with enzymes to eliminate all forms of Env but gp120/gp41 Env trimers. Particles are then surface-labeled with a streptavidin-fluorophore, e.g. streptavidin-phycoerythrin. Negative control particles bearing no Env and labeled with a different fluorophore can be used as controls for non-specific binding. Particles can then be used to probe memory B cells from HIV-1-infected patients known to harbor broadly neutralizing antibodies. B cells separated as single cells may then be expanded. B cell supernatants may then be tested for binding to recombinant Env gp120, gp41, particles, and gp120/gp41 trimers (the latter by native PAGE). They are also tested for activity in neutralization assays. IgG heavy and light chain genes may be PCR amplified and rescued into expression vectors. In a similar manner, particles bearing exclusively Env trimers, as well as soluble Env trimers can be used to probe B cell repertoires by alternative techniques involving flow cytometry, phage display, yeast display or other methods. B cells from animal or human vaccines that generate neutralizing responses could also be probed in a similar manner.

The following examples are intended to illustrate how to make and use the compositions and methods of this invention and are in no way to be construed as a limitation. Variations of the described examples will be apparent to those skilled in the art. All references cited herein are fully incorporated by reference in their entirety.

### Example 1

### Identifying the Aberrant Form of Env on VLPs

A major aberrant monomeric Env species on HIV-1 particles has been reported (65). Identifying this contaminant may help to devise strategies to eliminate it. VLP Env in SDS-PAGE was thus examined. SOS-VLPs produced in 293T cells and GnTI-cells, defective in N-acetyl glucosamine transferase I, were compared. The GnTI-mutant cell line can not convert immature oligomannoses into mature complex glycans (6). FIG. 3 shows SOS-VLPs from these two cell lines and monomeric gp120 resolved by reducing SDS-PAGE-Western. FIG. 3 parts A) and B) were probed with anti-gp120 or anti-gp41 mAb cocktails, respectively. Samples were (lanes 2, 4 & 6) or were not (lanes 1, 3 & 5) treated with endo H after denaturation. This removes high mannose glycans near their base, but does not affect complex glycans.

Multiple bands larger than 100 kDa were observed when SOS-VLPs were probed with gp120 Abs in lane 1 of FIG. 3A, one band of which may derive from the aberrant Env (65). In FIG. 3B, the same SOS-VLPs were probed with gp41 mAbs. The topmost band is mature gp160. The prominent band below it is another gp160 isoform. This latter band was also present in the GnTI-virus (FIG. 3A, lane 3). This band is termed gp160ER, where “ER” stands for endoplasmic reticulum—a compartment normally associated with immature glycans like those present in this species, as suggested by the endo H sensitivity analysis below. Gp160ER forms a sharp band, consistent with uniform glycans, contrasting with the more diffuse gp120 and gp160 bands of parent cells (FIG. 3A, lane 1) (23, 34, 63). A fainter band migrated just below gp160ER in GnTI-SOS-VLPs (FIG. 3A, lane 3). Probing with a gp41 cocktail (FIG. 3B, lane 3) revealed that this also contains gp41, so it is termed gp160GnTI-. Since high mannose glycans are smaller than complex glycans (˜1.5 vs ˜3 kDa), the gp160 from GnTI-cells is expected to be relatively small. The ˜90 kDa band of GnTI-Env in FIG. 3A, lane 3 did not stain with gp41 mAbs (FIG. 3B, lane 3), suggesting it to be a low molecular weight gp120, again consistent with smaller glycans.

Lanes 2, 4 & 6 of FIG. 3 show the effect of endo H on duplicate samples from lanes 1, 3 & 5, respectively. A consistent pattern was present in all four VLP quadrants: endo H mediated a dramatic drop in the gp160ER band size (FIGS. 3A & B, lanes 1-4). This contrasted to gp120, where a modest effect was observed (FIG. 3A lanes 1, 2, 5 & 6). The effect on mature gp160 was also modest (FIG. 3A lanes 1, 2). In further experiments, it was found that the gp160ER band also contaminates purified inactivated BaL and ADA viruses from the NIH (not shown & ref (65)).

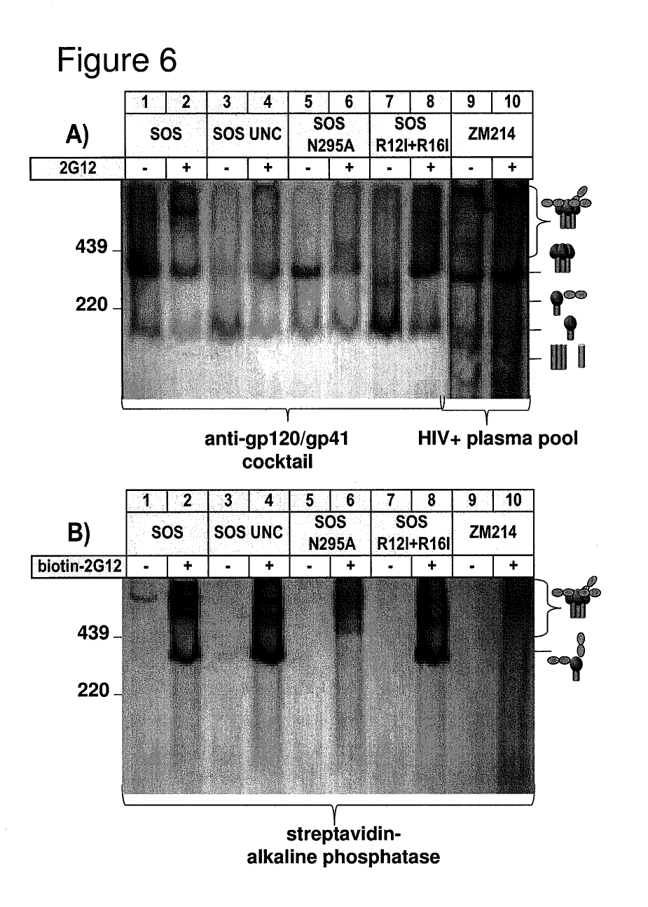

Whether gp160ER accounts for the monomer observed in BN-PAGE was next investigated. FIG. 6 shows that the SOS-VLP Env monomer in BN-PAGE exhibits two 2G12 binding sites. Here, SOS-VLPs, mutants thereof and ZM214 WT-VLPs produced in parent 293T cells were incubated with or without 2G12 or biotinylated 2G12, as indicated, then resolved by BN-PAGE-Western blot. Mutant SOS-VLPs included UNC (K510S+R511S), N295A mutant, and R12I+R16I. Blots were detected with either A) the full anti-gp120 and anti-gp41 cocktail, a clade C HIV+ plasma cocktail (for ZM214) or B) streptavidin-alkaline phosphatase. mAb 2G12-monomer binding caused an unusually large BN-PAGE shift from ˜140 kDa to ˜420 kDa (FIGS. 6A and B compare odd and even lanes; ref (19)). The N295A mutant knocks out 2G12 binding, as expected (FIG. 6, lanes 5 and 6). The R12I+R16I mutant was similar to the UNC mutants (FIG. 6, lanes 3, 4, 7 and 8) in being largely uncleaved gp160 monomer that again was shifted to a large extent by 2G12 (FIGS. 6A and B, compare lanes 3 and 7 to lanes 4 and 8). ZM214, like the N295A mutant of JR-FL lacks a 2G12 binding site and no shifts are observed (FIG. 6, lanes 9 and 10). As a monomer+IgG complex is expected to resolve at ˜300 kDa, it was difficult to explain the apparent ambiguity of double sized shifts mediated in several instances by 2G12 in FIG. 6. This suggests that the high mannose glycans unique to the outer domain of gp160ER form a second 2G12 binding site. Collectively, this data suggests that the monomer in BN-PAGE consists largely of gp160ER. These findings are consistent with a report that gp120 expressed in the presence of mannose analog kifunensine also binds two 2G12 molecules (86).

### Example 2

### Enzyme Digestion of Non-Functional Env

Denting the high mannose glycan shell of gp160ER might leave it vulnerable to proteases. To investigate, WT-VLPs were incubated with endo H at 37° C., followed by 1 h with various proteases in molar excesses and were then resolved by BN-PAGE-Western blot (FIG. 8). In some cases, PGNase F and neuraminase (NA) or a deglycosylation mixture (degly. Mix) and fucosidase (fuc.) were used as indicated in FIG. 8 Chymotrypsin effectively digested the uncleaved gp160 monomer, especially in the presence of endo H (FIG. 8, compare lanes 1, 3 and 4), but the gp120/gp41 trimer resisted digestion, in the same way that it resists non-neutralizing mAb binding. Cocktails of proteases were more effective than chymotrypsin alone (FIG. 8, compare lanes 4 and 6, where prot. K=proteinase K). Enzyme digests had a similar effect on SOS-VLPs (FIG. 9). Thus, embodiments of the present invention eliminate non-functional Env.

Whether enzymes can select soluble gp120/gp41 trimers from detergent-solubilized SOS-VLPs was determined. Solubilized Env gave a smear in BN-PAGE, perhaps due to aggregation (FIG. 14, lane 6). This was not resolved by endo H (FIG. 14, lane 2), but proteases all had a remarkable clarifying effect (FIG. 14, lanes 3, 4, 5, 7, 8 and 9). Digests were generally more effective against solubilized Env than particulate Env (compare FIGS. 8 & 14). In the case of chymotrypsin/endo H only authentic gp120/gp41 trimers remained (FIG. 14, lane 4). As above, proteinase K digested more gp120/gp41 trimer, while trypsin was only partially effective.

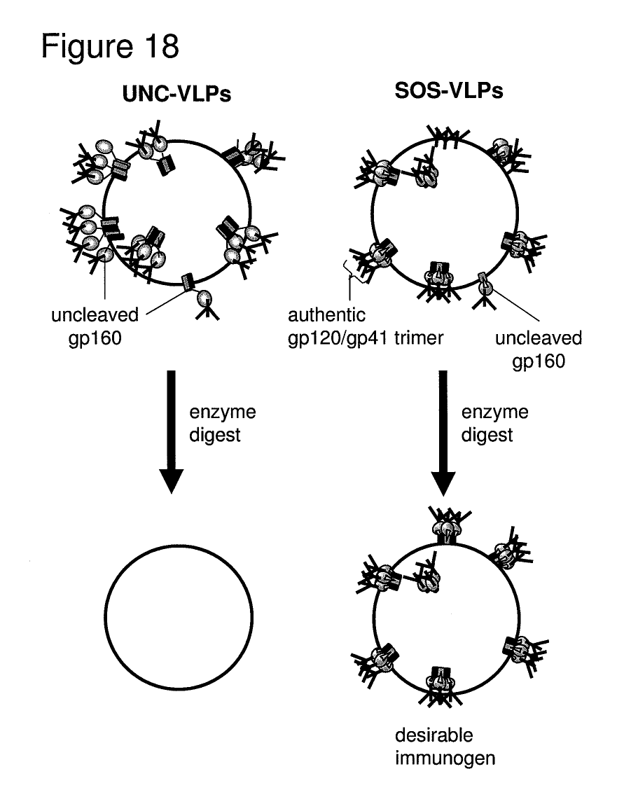

The applicability of these methods to Env-VLPs of differing neutralization sensitivities and clade was addressed. VLPs produced in parent or GnTI-cells bearing various JR-FL Envs, mutants thereof (FIG. 11) and other Env clones including those from different clades as well as monomeric recombinant gp120 (FIG. 12) were digested with endo H, chymotrypsin, trypsin, subtilisin and proteinase K and then resolved by BN-PAGE-Western blot. Blots were probed with the standard gp120/gp41 mAb cocktail, except ZM214, in which clade B V3 mAbs were substituted for non-clade B V3 mAbs 5.8C, 1.4E, 3074 and 3869 (FIG. 12). Like the SOS-VLP parent, various JR-FL based clones and diverse envelopes, for example WT-VLPs, ZM214-VLPs and M149A mutant SOS-VLPs were stripped of all but gp120/gp41 trimers (FIG. 11, 12). Conversely, GnTI-VLPs, A328G mutant SOS-VLPs, UNC-VLPs and monomeric gp120 Envs were digested almost indiscriminately (FIG. 11). The A328G mutant is highly neutralization sensitive. UNC-VLPs and gp120 are ‘promiscuous’ forms of Env. GnTI-gp120/gp41 trimers are decorated only with high mannose glycans and are therefore endo H-sensitive (see FIG. 6 of ref (6)). Note, however, the lower panel in FIG. 11, showing enhanced exposure of the upper panel. This reveals faint trimer bands for GnTI-virus even after digestion (FIG. 11, lane 8). Thus, in general, neutralization-resistant gp120/gp41 trimers also resist digestion, while neutralization-sensitive Envs are also protease-sensitive. An exception was the neutralization-sensitive M149A gp120/gp41 trimer that resisted the enzymes. This mutant is not as sensitive as A328G, and thus may be sufficiently compact to resist digestion. These observations regarding the effects of digests on VLPs are illustrated graphically in FIG. 18. Here, enzyme digestion of particles bearing mutant forms of JR-FL Env leaves only native Env trimers. UNC-VLPs are essentially completely cleared of all Env, while SOS-VLPs retain native trimers.

### Example 3

### The Fate of gp120/gp41 Trimer and Uncleaved gp160ER

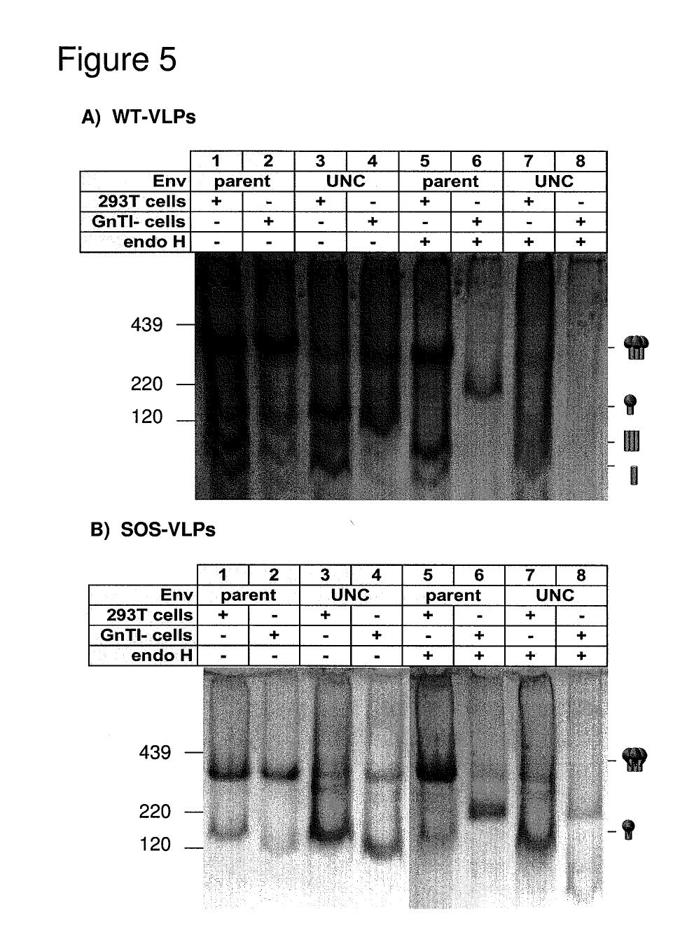

The fate of Env after digestion by SDS-PAGE-Western blot was determined. UNC WT-VLPs produced in 293T cells were enzyme digested, as indicated, then analyzed by SDS-PAGE-Western blot (FIG. 7). Blots were probed separately with anti-gp120 or anti-gp41 cocktails in parts A and B. Here, digests with endo H were overnight (O/N), those with deglycosylation mix/fucosidase (abbreviated degly. mix/fuc.) were for 2 h and proteinase K digests were for 1 hour. In some cases, VLPs were boiled for 5 minutes before digests. Washes were performed between each digest reaction unless indicated. The maximum possible enzyme effects were assessed by denaturing/reducing UNC-VLPs prior to digestion. As in FIG. 3, endo H profoundly affected gp160ER, but had less effect on gp120 and gp160 (FIG. 7, lanes 1 & 2). Chymotrypsin completely digested all bands, as expected (FIG. 7, lane 3). Next, the effects of enzymes on intact VLPs and the effects of washing out or leaving in the enzyme upon addition of SDS loading buffer were assessed. Endo H/chymotrypsin digests without washing led to the complete removal of gp160ER, but had little effect on gp120 and gp41 stumps that derive from authentic gp120/gp41 trimers (FIG. 7, lane 5), consistent with the selective digestion of uncleaved gp160 monomer in BN-PAGE (FIG. 5-8). When a wash step was included, however, some gp160ER remained (FIG. 7, lane 6). Thus, as observed above (FIGS. 8 and 14), digestion is more effective after solubilization. Endo H digestion alone led to the appearance of smaller bands (FIG. 7, lane 4), consisting of glycan shell-dented gp160ER. The fact that these bands were missing when chymotrypsin was added (lane 8), suggests that they are protease sensitive. In line with this idea, chymotrypsin alone was less effective at digesting the fully glycosylated gp160ER (FIG. 7, lane 7).

### Example 4

### Functional and Antigenic Consequences of Digestion

The effects of VLP digestion on epitope exposure were assessed by virus capture using various mAbs (FIG. 15). SOS-VLPs were treated overnight with endo H, for 1 h with chymotrypsin and assayed for capture by various mAbs. The percentage of capture of treated vs mock virus is shown for each mAb. Capture of digested virus was generally lower than that of mock virus. However, capture by non-neutralizing mAbs was far more markedly affected, consistent with the selective removal of gp160ER (FIG. 15). That neutralizing mAb capture was also affected is consistent with the idea that these mAbs capture in part via gp160ER and its loss therefore affects capture. The fact that the captured virus still readily infects the CF2.CD4.CCR5 target cells (to 100% of mock, FIG. 13) further indicates that gp120/gp41 trimers are unaffected by the enzyme treatments. More recent analysis (FIG. 21) of JR-FL WT E168K mutant VLPs following digestion in thin walled tubes by proteases trypsin, chymotrypsin, proteinase K and subtilisin (no endo H), show that neutralizing antibody epitopes (filled symbols) are better retained than non-neutralizing epitopes (open diamonds). This is consistent with the selective removal of non-functional Env, to which non-neutralizing mAbs bind.

The effect of digests on mAb-gp120/gp41 trimer binding in BN-PAGE was investigated (FIG. 16). Here, undigested WT-VLPs (FIG. 16A) or WT “trimer VLPs” (FIG. 16B) or E168K+N189A WT “trimer VLPs” (FIG. 16C). Trimer VLPs in FIGS. 16B and C were digested with a combination of trypsin, chymotrypsin, proteinase K and subtilisin were incubated for 3 h at 37° C. with various mAbs at 30 μg/ml. VLPs were washed and resolved by BN-PAGE-Western blot. Protease digestion (trypsin, chymotrypsin, proteinase K and subtilisin in thin walled tubes overnight at 37° C.) of WT-VLPs had a remarkable clarifying effect (compare FIGS. 16A & B). NAb binding to authentic gp120/gp41 trimers in this assay (see FIG. 2 of (7)), exclusively tracks with neutralization (6, 7, 9, 19-21, 42, 43, 65, 66, 96). Typically, mAb-gp120/gp41 trimer complexes form smears, perhaps because IgGs are flexible (19). Therefore, NAb binding was measured semi-quantitatively as a depletion of unliganded gp120/gp41 trimer as it forms mAb complexes. However, an exception is mAb 2G12 mediates well-defined gp120/gp41 trimer shifts visible as a ladder, perhaps due to its uniquely rigid structure (14). The 2G12 ladder became very clear. Neutralizing mAbs in lanes 2-8 shift WT trimers, but non-neutralizing mAbs (lanes 9-13) do not. Note that PG9 and PG16 do not shift WT trimers, because they only neutralize (or shift trimers) when an E168K mutant is introduced (Compare FIGS. 16B and C). Importantly, mAbs 15e and 39F remained unable to bind gp120/gp41 trimers. The prominent gp41 stump band induced by sCD4 (FIG. 16B, C) is likely to be caused by gp120 shedding.

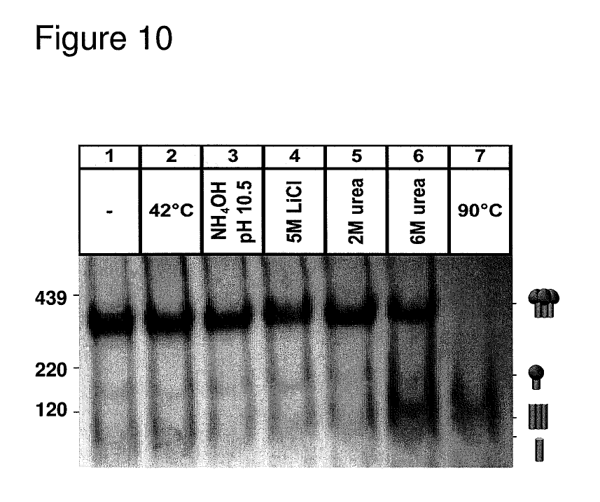

The effects of digests on the stability of VLP Env trimers was assessed (FIG. 10). WT-VLPs were digested with endo H, chymotrypsin, trypsin, subtilisin and proteinase K (as in FIG. 8, lane 6) and trimer stability was then determined in the face of the harsh conditions indicated. Samples were then analyzed by BN-PAGE-Western blot and probed with anti-gp120/gp41 mAb cocktails. The trimer on VLPs retained after digestion was stable to various conditions such as high salt and extreme pH (FIG. 10, compare lane 1 to lanes 2-6). However, trimers were sensitive to high temperature (FIG. 10, compare lane 1 to lane 7)

### Example 5

### Evaluating Pure Authentic gp120/gp41 Trimer Immunogens in Rabbits

Animal Numbers, VLP Purification, Dose, Route and Schedule.

Preliminary immunogenicity studies using VLP immunogens have suggested an overwhelming preoccupation of B cells with aberrant Env. Embodiments of the present invention provide a solution this problem by making pure gp120/gp41 trimer immunogens. 20 rabbits/year, 4/group, for a total of up to 100 over 5 years are immunized. Supernatants are processed by low speed centrifugation to clear cells, filtration and then high-speed centrifugation. Previously, immunized guinea pigs, rabbits and RMs have been immunized with VLPs produced in this manner ((20)). ˜1 L of VLP supernatant/week, enough for 3 rabbit shots (300 μl of 1000×) are produced. VLPs are inactivated by 1 mM AT-2 (20), adjuvant co-formulated and administered 3 times by IM injection at 6 week intervals, with bleeds on the day of immunization and 2 weeks thereafter.

Initial Immunogens.

Preliminary studies indicated that adjuvants can dramatically impact Ab specificity (46, 55, 93, 98). An initial comparison of adjuvants would help identify the most effective for further studies. Untreated and “pure gp120/gp41 trimer” WT-VLPs are compared using 3 adjuvants: Carbopol, QS21, and Ribi in a total of 24 animals. Carbopol is a polyanionic carbomer that promotes Thl responses and has been evaluated in a variety of veterinary vaccine settings (46). Ribi is an oil-in-water emulsion (0.2% Tween80) containing bacterial cell wall skeleton (a TLR2 agonist) and MPLA. As necessary, protease inhibitors are added and later removed before immunization by washing.

### Serum Analysis

Gp120 and gp41 ELISA.

NAbs may cross-react with monomeric gp120 or gp41, or recognize quaternary epitopes. Therefore, a lack of serum anti-gp120 and gp41 titers may be an indication of success.

Reactivity with Bald VLPs.

Antibody responses to non-Env components of VLPs is monitored by ELISA using bald VLPs (the same as WT-VLPs, but with no Env on their surface) to help gauge efforts to adsorb the activity, or to eliminate it by IgG masking or the use of soluble gp120/gp41 trimer immunogens.

Neutralization.

To assess progress in eliciting nAbs, an algorithm using BN-PAGE and 2 neutralization assays has been developed (20). Each assay is performed 3 times in duplicate. IC50 titers of >1:100 against JR-FL would be a significant lead. Mann-Whitney tests will determine significance (4). NAb breadth against primary viruses of clade B and C reference panels is determined (53).

i) BN-PAGE.

BN-PAGE avoids the effects of “anti-cell” Abs that can plague neutralization assays (2, 20, 59, 99). Two recent findings have led to dramatic improvements: a) eliminating non-functional Env leads to unprecedented clarity (FIG. 16, 20), b) modified mAb-virus incubations increase sensitivity.

ii) Neutralization Assays.

Neutralization on CF2 and TZM-bl target cells (20) is evaluated. CF2 cells are of canine origin and may help limit non-specific effects due to Abs against HEK293T producer cells (21). Clade B and C reference viruses are evaluated (69). The use of SIVmac239, MuLV and VSV-G control viruses will help gauge any nonspecific effects (21). As necessary, >90% of the anti-cell activity is adsorbed on compacted 293T cells and monitored by flow cytometry (20). Another approach to confirm specific neutralization is to fractionate sera on gp120 or MPER peptides (7, 9). While nAbs may bind gp120 or MPER peptides, anti-cell Abs would be removed in the flow through.

Mapping.

Fractionations with gp120 and MPER peptides may also help in mapping. A panel of more than 300 JR-FL point mutants have been made. These can be used for mapping by neutralization or BN-PAGE, as described in refs (7, 9). If non-neutralizing anti-Env Ab responses are generated, mapping by competitive VLP ELISA or virus capture will provide information on how immunogens might be improved (20).

### Example 6

### Evaluating Pure Authentic gp120/gp41 Trimer Immunogens in Macaques

Primate studies are performed. 16 rhesus macaques (RMs) are immunized in an initial experiment, 12 in a follow up experiment and 16 in a third experiment for a total of 44. Immune groups will consist of 4 animals. Most animals will receive 4 monthly ID inoculations and 10 ml bleeds on day of inoculation and 2 weeks thereafter. The studies outlined below assume that VLPs will be the immunogens. However, if soluble gp120/gp41 trimers show demonstrable advantages in rabbits, they can be substituted for the equivalent VLPs. Serum and mAb neutralization against candidate SHIVs in TZM-bl cells and rhesus PBMCs are examined (38, 68, 72). This will help in i) selecting an appropriate challenge SHIV based on a moderate neutralization resistance profile, ii) gauging any anti-cell Ab effects that might be addressed by IgG masking or the use of soluble gp120/gp41 trimer immunogens, iii) predicting if serum neutralization titers are likely to be protective (67, 103).

Initial RM immunogens will depend on the outcome of rabbit studies. An optimal adjuvant (at least for rabbits) should be clear and will be adopted for primates. The best performing immunogens and a matched mock, e.g. digested and untreated JR-FL SOS-VLPs (4 RMs/group) are compared. A challenge is that Env immunogens may complex with endogenous CD4 (30), leading to unwanted exposure of V3 and CD4i epitopes. Therefore, in a third group (n=4 RMs), immunogens that eliminate CD4 binding but have no effect on b12 or other nAb binding, e.g. mutants D368E, W427A, or D457E, are tested and confirmed they ablate macaque sCD4 binding. None of these cause global changes in neutralization sensitivity, as evidenced in BN-PAGE. Mutant selection will depend on expression and the ability of the gp120/gp41 trimer to survive digests. An alternative approach may be to use gp120/gp41 trimers stabilized by a lateral disulfide. These gp120/gp41 trimers may exhibit a rigid structure that can not undergo CD4 binding rearrangements (58)- and may also be better immunogens. A fourth group of animals (n=4 RMs) will be immunized with Naked-VLPs. As needed, purified IgG from these animals will help mask the development of anti-cell Abs in subsequent animals and may also augment anti-gp120/gp41 trimer responses.

Animals are immunized with VLPs bearing Env matched to the prospective challenge virus. SHIV selection will be based in part on the efficient expression of the matched Env immunogen. Candidate SHIVs e.g. BaL and AD8 are R5-tropic, representative of viral transmission (38, 68, 72). Thus, immunogens may be:

BaL-VLPs with CD4 knockout mutation, untreated, & complexed with Naked-VLP IgG

BaL-VLPs with CD4 knockout mutation, digested, & complexed with Naked-VLP IgG

BaL-VLPs with CD4 knockout mutation, digested but no Naked-VLP IgG.

The protective efficacy of the lead immunogen against a homologous SHIV challenge will be tested, using animals lacking protective alleles (38). A 2nd group of animals receive mock VLPs to assess any protective effects of cellular responses. A 3rd group will be naïve controls. The first 2 groups will consist of 6 animals and the control group will consist of 4 naïve animals, as in ref (38). As necessary, immunogens are complexed with Naked-VLP IgG.

SHIV challenge. Before challenge, the neutralizing IC50s of the penultimate bleed against the challenge virus are assessed. Two weeks after the end of immunizations, animals are challenged intrarectally with a homologous SHIV at 300 TCID50. Standard bleeds are taken at 7, 10, 14, 21, 28, 35, 42, 60, 90, 120, 180, 240, 300, and 360 days. Viral RNA (mac239 Gag) is quantified by real time PCR. Sera is analyzed. CD4 T cells are measured by flow cytometry. Any post-challenge changes in binding and nAb titers are monitored. Statistical analyses will determine protective efficacy, as in ref (38).

### REFERENCES

1. Abrahamyan, L. G., R. M. Markosyan, J. P. Moore, F. S. Cohen, and G. B. Melikyan. 2003. Human immunodeficiency virus type 1 Env with an intersubunit disulfide bond engages coreceptors but requires bond reduction after engagement to induce fusion. J Virol 77:5829-36.
2. Arthur, L. O., J. W. Bess, Jr., R. C. Sowder, 2nd, R. E. Benveniste, D. L. Mann, J. C. Chermann, and L. E. Henderson. 1992. Cellular proteins bound to immunodeficiency viruses: implications for pathogenesis and vaccines. Science 258:1935-8.
3. Beddows, S., M. Franti, A. K. Dey, M. Kirschner, S. P. Iyer, D.C. Fisch, T. Ketas, E. Yuste, R. C. Desrosiers, P. J. Klasse, P. J. Maddon, W. C. Olson, and J. P. Moore. 2006. A comparative immunogenicity study in rabbits of disulfide-stabilized, proteolytically cleaved, soluble trimeric human immunodeficiency virus type 1 gp140, trimeric cleavage-defective gp140 and monomeric gp120. Virology.
4. Beddows, S., N. Schulke, M. Kirschner, K. Barnes, M. Franti, E. Michael, T. Ketas, R. W. Sanders, P. J. Maddon, W. C. Olson, and J. P. Moore. 2005. Evaluating the immunogenicity of a disulfide-stabilized, cleaved, trimeric form of the envelope glycoprotein complex of human immunodeficiency virus type 1. J Virol 79:8812-27.
5. Billington, J., T. P. Hickling, G. H. Munro, C. Halai, R. Chung, G. G. Dodson, and R. S. Daniels. 2007. Stability of a receptor-binding active human immunodeficiency virus type 1 recombinant gp140 trimer conferred by intermonomer disulfide bonding of the V3 loop: differential effects of protein disulfide isomerase on CD4 and coreceptor binding. J Virol 81:4604-14.
6. Binley, J., Y. E. Ban, E. T. Crooks, D. Eggink, K. Osawa, W. R. Schief, and R. W. Sanders. 2010. Role of Complex Carbohydrates in Human Immunodeficiency Virus Type 1 Infection and Resistance to Antibody Neutralization. J.Virol. 84:5637-5655.
7. Binley, J. M. 2009. Specificities of Broadly Neutralizing anti-HIV-1 sera. Current Opinion in HIV and AIDS 4:364-372.
8. Binley, J. M., C. S. Cayanan, C. Wiley, N. Schulke, W. C. Olson, and D. R. Burton. 2003. Redox-triggered infection by disulfide-shackled human immunodeficiency virus type 1 pseudovirions. J Virol 77:5678-84.
9. Binley, J. M., E. A. Lybarger, E. T. Crooks, M. S. Seaman, E. S. Gray, K. L. Davis, J. M. Decker, D. Wycuff, L. Harris, N. Hawkins, B. Wood, C. Nathe, D. Richman, G. D. Tomaras, F. Bibollet-Ruche, J. E. Robinson, L. Morris, G. M. Shaw, D.C. Montefiori, and J. R. Mascola. 2008. Profiling the Specificity of Neutralizing Antibodies in a Large Panel of HIV-1 Plasmas from Subtype B and C Chronic Infections. J Virol 82:11651-11668.
10. Binley, J. M., R. W. Sanders, B. Clas, N. Schuelke, A. Master, Y. Guo, F. Kajumo, D. J. Anselma, P. J. Maddon, W. C. Olson, and J. P. Moore. 2000. A recombinant human immunodeficiency virus type 1 envelope glycoprotein complex stabilized by an intermolecular disulfide bond between the gp120 and gp41 subunits is an antigenic mimic of the trimeric virion-associated structure. J Virol 74:627-43.
11. Blay, W. M., T. Kasprzyk, L. Misher, B. A. Richardson, and N. L. Haigwood. 2007. Mutations in envelope gp120 can impact proteolytic processing of the gp160 precursor and thereby affect neutralization sensitivity of human immunodeficiency virus type 1 pseudoviruses. J Virol 81:13037-49.
12. Bultmann, A., J. Eberle, and J. Haas. 2000. Ubiquitination of the human immunodeficiency virus type 1 env glycoprotein. J Virol 74:5373-6.
13. Burton, D. R., R. L. Stanfield, and I. A. Wilson. 2005. Antibody vs. HIV in a clash of evolutionary titans. Proc Natl Acad Sci USA 102:14943-8.
14. Calarese, D. A., C. N. Scanlan, M. B. Zwick, S. Deechongkit, Y. Mimura, R. Kunert, P. Zhu, M. R. Wormald, R. L. Stanfield, K. H. Roux, J. W. Kelly, P. M. Rudd, R. A. Dwek, H. Katinger, D. R. Burton, and I. A. Wilson. 2003. Antibody domain exchange is an immunological solution to carbohydrate cluster recognition. Science 300:2065-71.
15. Chen, L., Y. D. Kwon, T. Zhou, X. Wu, S. O'Dell, L. Cavacini, A. J. Hessell, M. Pancera, M. Tang, L. Xu, Z. Y. Yang, M. Y. Zhang, J. Arthos, D. R. Burton, D. S. Dimitrov, G. J. Nabel, M. R. Posner, J. Sodroski, R. Wyatt, J. R. Mascola, and P. D. Kwong. 2009. Structural basis of immune evasion at the site of CD4 attachment on HIV-1 gp120. Science 326:1123-7.
16. Chen, X., M. T. Rock, J. Hammonds, J. Tartaglia, A. Shintani, J. Currier, B. Slike, J. E. Crowe, Jr., M. Marovich, and P. Spearman. 2005. Pseudovirion particle production by live poxvirus human immunodeficiency virus vaccine vector enhances humoral and cellular immune responses. J Virol 79:5537-47.
17. Cho, M. W., Y. B. Kim, M. K. Lee, K. C. Gupta, W. Ross, R. Plishka, A. Buckler-White, T. Igarashi, T. Theodore, R. Byrum, C. Kemp, D.C. Montefiori, and M. A. Martin. 2001. Polyvalent envelope glycoprotein vaccine elicits a broader neutralizing antibody response but is unable to provide sterilizing protection against heterologous Simian/human immunodeficiency virus infection in pigtailed macaques. J Virol 75:2224-34.
18. Corti, D., D. Pinna, F. Vanzetta, S. Balla, H. Dreja, E. O'Sullivan, C. Pade, A. Hinz, H. Langedijk, W. Weissenhorn, W. Janssens, A. McKnight, F. Sallusto, R. A. Weiss, and A. Lanzavecchia. 2008. A Panel of HIV-1 Neutralizing Antibodies Isolated from non-clade B HIV-1 Infected Donors. AIDS Vaccine 08 Conference, abstract S01-05.
19. Crooks, E. T., P. Jiang, M. Franti, S. Wong, M. B. Zwick, J. A. Hoxie, J. E. Robinson, P. L. Moore, and J. M. Binley. 2008. Relationship of HIV-1 and SIV envelope glycoprotein trimer occupation and neutralization. Virology 377:364-378.
20. Crooks, E. T., P. L. Moore, M. Franti, C. S. Cayanan, P. Zhu, P. Jiang, R. P. de Vries, C. Wiley, I. Zharkikh, N. Schulke, K. H. Roux, D.C. Montefiori, D. R. Burton, and J. M. Binley. 2007. A comparative immunogenicity study of HIV-1 virus-like particles bearing various forms of envelope proteins, particles bearing no envelope and soluble monomeric gp120. Virology 366:245-62.
21. Crooks, E. T., P. L. Moore, D. Richman, J. Robinson, J. A. Crooks, M. Franti, N. Schulke, and J. M. Binley. 2005. Characterizing anti-HIV monoclonal antibodies and immune sera by defining the mechanism of neutralization. Hum Antibodies 14:101-13.
22. Cubas, R., S. Zhang, S. Kwon, E. M. Sevick-Muraca, M. Li, C. Chen, and Q. Yao. 2009. Virus-like particle (VLP) lymphatic trafficking and immune response generation after immunization by different routes. J Immunother 32:118-28.
23. Cutalo, J. M., L. J. Deterding, and K. B. Tomer. 2004. Characterization of glycopeptides from HIV-I(SF2) gp120 by liquid chromatography mass spectrometry. J Am Soc Mass Spectrom 15:1545-55.
24. Davis, S. J., E. A. Davies, A. N. Barclay, S. Daenke, D. L. Bodian, E. Y. Jones, D. I. Stuart, T. D. Butters, R. A. Dwek, and P. A. van der Merwe. 1995. Ligand binding by the immunoglobulin superfamily recognition molecule CD2 is glycosylation-independent. J Biol Chem 270:369-75.
25. Doria-Rose, N. A., R. M. Klein, M. G. Daniels, S. O'Dell, M. Nason, A. Lapedes, T. Bhattacharya, S. A. Migueles, R. T. Wyatt, B. T. Korber, J. R. Mascola, and M. Connors. 2010. Breadth of human immunodeficiency virus-specific neutralizing activity in sera: clustering analysis and association with clinical variables. J Virol 84:1631-6.
26. Earl, P. L., C. C. Broder, D. Long, S. A. Lee, J. Peterson, S. Chakrabarti, R. W. Doms, and B. Moss. 1994. Native oligomeric human immunodeficiency virus type 1 envelope glycoprotein elicits diverse monoclonal antibody reactivities. J Virol 68:3015-26.
27. Earl, P. L., R. W. Doms, and B. Moss. 1990. Oligomeric structure of the human immunodeficiency virus type 1 envelope glycoprotein. Proc Natl Acad Sci USA 87:648-52.
28. Eggink, D., M. Melchers, M. Wuhrer, T. van Montfort, A. K. Dey, B. Naaijkens, K. B. David, V. LeDouce, A. M. Deelder, K. Kang, W. C. Olson, B. Berkhout, C. H. Hokke, J. P. Moore, and R. W. Sanders. 2010. Lack of complex N-glycans on HIV-1 envelope glycoproteins preserves protein conformation and entry function. Virology 401:236-247.
29. Finzi, A., S. H. Xiang, B. Pacheco, L. Wang, J. Haight, A. Kassa, B. Danek, M. Pancera, P. D. Kwong, and J. Sodroski. 2010. Topological layers in the HIV-1 gp120 inner domain regulate gp41 interaction and CD4-triggered conformational transitions. Mol Cell 37:656-67.
30. Forsell, M. N., B. Dey, A. Morner, K. Svehla, S. O'Dell, C. M. Hogerkorp, G. Voss, R. Thorstensson, G. M. Shaw, J. R. Mascola, G. B. Karlsson Hedestam, and R. T. Wyatt. 2008. B cell recognition of the conserved HIV-1 co-receptor binding site is altered by endogenous primate CD4. PLoS Pathog 4:e1000171.
31. Fouts, T. R., J. M. Binley, A. Trkola, J. E. Robinson, and J. P. Moore. 1997. Neutralization of the human immunodeficiency virus type 1 primary isolate JR-FL by human monoclonal antibodies correlates with antibody binding to the oligomeric form of the envelope glycoprotein complex. J Virol 71:2779-85.
32. Gao, F., B. T. Korber, E. Weaver, H. X. Liao, B. H. Hahn, and B. F. Haynes. 2004. Centralized immunogens as a vaccine strategy to overcome HIV-1 diversity. Expert Rev Vaccines 3:S161-8.
33. Gao, F., E. A. Weaver, Z. Lu, Y. Li, H. X. Liao, B. Ma, S. M. Alam, R. M. Scearce, L. L. Sutherland, J. S. Yu, J. M. Decker, G. M. Shaw, D.C. Montefiori, B. T. Korber, B. H. Hahn, and B. F. Haynes. 2005. Antigenicity and immunogenicity of a synthetic human immunodeficiency virus type 1 group m consensus envelope glycoprotein. J Virol 79:1154-63.
34. Go, E. P., J. Irungu, Y. Zhang, D. S. Dalpathado, H. X. Liao, L. L. Sutherland, S. M. Alam, B. F. Haynes, and H. Desaire. 2008. Glycosylation site-specific analysis of HIV envelope proteins (JR-FL and CON-S) reveals major differences in glycosylation site occupancy, glycoform profiles, and antigenic epitopes' accessibility. J Proteome Res 7:1660-74.
35. Hammonds, J., X. Chen, T. Fouts, A. DeVico, D. Montefiori, and P. Spearman. 2005. Induction of neutralizing antibodies against human immunodeficiency virus type 1 primary isolates by Gag-Env pseudovirion immunization. J Virol 79:14804-14.
36. Hammonds, J., X. Chen, X. Zhang, F. Lee, and P. Spearman. 2007. Advances in methods for the production, purification, and characterization of HIV-1 Gag-Env pseudovirion vaccines. Vaccine 25:8036-48.
37. Haynes, B. F., and D.C. Montefiori. 2006. Aiming to induce broadly reactive neutralizing antibody responses with HIV-1 vaccine candidates. Expert Rev Vaccines 5:347-63.
38. Hessell, A. J., E. G. Rakasz, D. M. Tehrani, M. Huber, K. L. Weisgrau, G. Landucci, D. N. Forthal, W. C. Koff, P. Poignard, D. I. Watkins, and D. R. Burton. 2010. Broadly neutralizing monoclonal antibodies 2F5 and 4E10 directed against the human immunodeficiency virus type 1 gp41 membrane-proximal external region protect against mucosal challenge by simian-human immunodeficiency virus SHIVBa-L. J Virol 84:1302-13.
39. Hurwitz, J. L., K. S. Slobod, T. D. Lockey, S. Wang, T. H. Chou, and S. Lu. 2005. Application of the polyvalent approach to HIV-1 vaccine development. Curr Drug Targets Infect Disord 5:143-56.
40. Jia, B., S. K. Ng, M. Q. DeGottardi, M. Piatak, E. Yuste, A. Carville, K. G. Mansfield, W. Li, B. A. Richardson, J. D. Lifson, and D. T. Evans. 2009. Immunization with single-cycle SIV significantly reduces viral loads after an intravenous challenge with SIV(mac)239. PLoS Pathog 5:el 000272.
41. Johnston, M. I., and A. S. Fauci. 2007. An HIV vaccine—evolving concepts. N Engl J Med 356:2073-81.
42. Kang, K., S. Andjelic, J. M. Binley, E. T. Crooks, M. Franti, S. P. Iyer, G. P. Donovan, A. K. Dey, P. Zhu, K. H. Roux, D. N. Fisch, R. J. Durso, J. Gao, A. M. Cupo, A. A. Ouatarra, V. M. Seetharaman, L. D. Kraweic, T. Parsons, P. J. Maddon, J. P. Moore, and W. C. Olson. 2008. Structure and Immunogenicity of a Cleaved, Stabilized Envelope Trimer Derived from Subtype A HIV-1. Virology submitted November 2008.
43. Kang, Y. K., S. Andjelic, J. M. Binley, E. T. Crooks, M. Franti, S. P. Iyer, G. P. Donovan, A. K. Dey, P. Zhu, K. H. Roux, R. J. Durso, T. F. Parsons, P. J. Maddon, J. P. Moore, and W. C. Olson. 2009. Structural and immunogenicity studies of a cleaved, stabilized envelope trimer derived from subtype A HIV-1. Vaccine 27:5120-32.
44. Kassa, A., A. Finzi, M. Pancera, J. R. Courter, A. B. Smith, 3rd, and J. Sodroski. 2009. Identification of a human immunodeficiency virus type 1 envelope glycoprotein variant resistant to cold inactivation. J Virol 83:4476-88.
45. Kassa, A., N. Madani, A. Schon, H. Haim, A. Finzi, S. H. Xiang, L. Wang, A. Princiotto, M. Pancera, J. Courter, A. B. Smith, 3rd, E. Freire, P. D. Kwong, and J. Sodroski. 2009. Transitions to and from the CD4-Bound Conformation Are Modulated by a Single-Residue Change in the Human Immunodeficiency Virus Type 1 gp120 Inner Domain. J Virol 83:8364-78.
46. Krashias, G., A. K. Simon, F. Wegmann, W. L. Kok, L. P. Ho, D. Stevens, J. Skehel, J. L. Heeney, A. E. Moghaddam, and Q. J. Sattentau. 2010. Potent adaptive immune responses induced against HIV-1 gp140 and influenza virus HA by a polyanionic carbomer. Vaccine 28:2482-9.
47. Kuate, S., C. Stahl-Hennig, H. Stoiber, G. Nchinda, A. Floto, M. Franz, U. Sauermann, S. Bredl, L. Deml, R. Ignatius, S. Norley, P. Racz, K. Tenner-Racz, R. M. Steinman, R. Wagner, and K. Uberla. 2006. Immunogenicity and efficacy of immunodeficiency virus-like particles pseudotyped with the G protein of vesicular stomatitis virus. Virology 351:133-44.
48. Kwong, P. D., M. L. Doyle, D. J. Casper, C. Cicala, S. A. Leavitt, S. Majeed, T. D. Steenbeke, M. Venturi, I. Chaiken, M. Fung, H. Katinger, P. W. Parren, J. Robinson, D. Van Ryk, L. Wang, D. R. Burton, E. Freire, R. Wyatt, J. Sodroski, W. A. Hendrickson, and J. Arthos. 2002. HIV-1 evades antibody-mediated neutralization through conformational masking of receptor-binding sites. Nature 420:678-82.
49. LaCasse, R. A., K. E. Follis, M. Trahey, J. D. Scarborough, D. R. Littman, and J. H. Nunberg. 1999. Fusion-competent vaccines: broad neutralization of primary isolates of HIV. Science 283:357-62.
50. Law, M., R. M. Cardoso, I. A. Wilson, and D. R. Burton. 2007. Antigenic and immunogenic study of membrane-proximal external region-grafted gp120 antigens by a DNA prime-protein boost immunization strategy. J Virol 81:4272-85.
51. Leaman, D. P., H. Kinkead, and M. B. Zwick. 2010. In-solution virus capture assay helps deconstruct heterogeneous antibody recognition of human immunodeficiency virus type 1. J Virol 84:3382-95.
52. Li, B., J. M. Decker, R. W. Johnson, F. Bibollet-Ruche, X. Wei, J. Mulenga, S. Allen, E. Hunter, B. H. Hahn, G. M. Shaw, J. L. Blackwell, and C. A. Derdeyn. 2006. Evidence for potent autologous neutralizing antibody titers and compact envelopes in early infection with subtype C human immunodeficiency virus type 1. J Virol 80:5211-8.
53. Li, M., F. Gao, J. R. Mascola, L. Stamatatos, V. R. Polonis, M. Koutsoukos, G. Voss, P. Goepfert, P. Gilbert, K. M. Greene, M. Bilska, D. L. Kothe, J. F. Salazar-Gonzalez, X. Wei, J. M. Decker, B. H. Hahn, and D.C. Montefiori. 2005. Human immunodeficiency virus type 1 env clones from acute and early subtype B infections for standardized assessments of vaccine-elicited neutralizing antibodies. J Virol 79:10108-25.
54. Li, M., J. F. Salazar-Gonzalez, C. A. Derdeyn, L. Morris, C. Williamson, J. E. Robinson, J. M. Decker, Y. Li, M. G. Salazar, V. R. Polonis, K. Mlisana, S. A. Karim, K. Hong, K. M. Greene, M. Bilska, J. Zhou, S. Allen, E. Chomba, J. Mulenga, C. Vwalika, F. Gao, M. Zhang, B. T. Korber, E. Hunter, B. H. Hahn, and D.C. Montefiori. 2006. Genetic and neutralization properties of subtype C human immunodeficiency virus type 1 molecular env clones from acute and early heterosexually acquired infections in Southern Africa. J Virol 80:11776-90.
55. Li, Y., K. Svehla, N. L. Mathy, G. Voss, J. R. Mascola, and R. Wyatt. 2006. Characterization of antibody responses elicited by human immunodeficiency virus type 1 primary isolate trimeric and monomeric envelope glycoproteins in selected adjuvants. J Virol 80:1414-26.
56. Liao, H. X., M. C. Levesque, A. Nagel, A. Dixon, R. Zhang, E. Walter, R. Parks, J. Whitesides, D. J. Marshall, K. K. Hwang, Y. Yang, X. Chen, F. Gao, S. Munshaw, T. B. Kepler, T. Denny, M. A. Moody, and B. F. Haynes. 2009. High-throughput isolation of immunoglobulin genes from single human B cells and expression as monoclonal antibodies. J Virol Methods 158:171-9.
57. Lifson, J. D., J. L. Rossio, M. Piatak, Jr., J. Bess, Jr., E. Chertova, D. K. Schneider, V. J. Coalter, B. Poore, R. F. Kiser, R. J. Imming, A. J. Scarzello, L. E. Henderson, W. G. Alvord, V. M. Hirsch, R. E. Benveniste, and L. O. Arthur. 2004. Evaluation of the safety, immunogenicity, and protective efficacy of whole inactivated simian immunodeficiency virus (SIV) vaccines with conformationally and functionally intact envelope glycoproteins. AIDS Res Hum Retroviruses 20:772-87.
58. Liu, J., A. Bartesaghi, M. J. Borgnia, G. Sapiro, and S. Subramaniam. 2008. Molecular architecture of native HIV-1 gp120 trimers. Nature 455:109-13.
59. McBurney, S. P., K. R. Young, and T. M. Ross. 2007. Membrane embedded HIV-1 envelope on the surface of a virus-like particle elicits broader immune responses than soluble envelopes. Virology 358:334-46.
60. McKenna, P. M., R. J. Pomerantz, B. Dietzschold, J. P. McGettigan, and M. J. Schnell. 2003. Covalently linked human immunodeficiency virus type 1 gp120/gp41 is stably anchored in rhabdovirus particles and exposes critical neutralizing epitopes. J Virol 77:12782-94.
61. Means, R. E., and R. C. Desrosiers. 2000. Resistance of native, oligomeric envelope on simian immunodeficiency virus to digestion by glycosidases. J Virol 74:11181-90.
62. Mische, C. C., W. Yuan, B. Strack, S. Craig, M. Farzan, and J. Sodroski. 2005. An alternative conformation of the gp41 heptad repeat 1 region coiled coil exists in the human immunodeficiency virus (HIV-1) envelope glycoprotein precursor. Virology 338:133-43.
63. Mizuochi, T., T. J. Matthews, M. Kato, J. Hamako, K. Titani, J. Solomon, and T. Feizi. 1990. Diversity of oligosaccharide structures on the envelope glycoprotein gp120 of human immunodeficiency virus 1 from the lymphoblastoid cell line H9. Presence of complex-type oligosaccharides with bisecting N-acetylglucosamine residues. J Biol Chem 265:8519-24.
64. Montefiori, D., Q. Sattentau, J. Flores, J. Esparza, and J. Mascola. 2007. Antibody-based HIV-1 vaccines: recent developments and future directions. PLoS Med 4:e348.
65. Moore, P. L., E. T. Crooks, L. Porter, P. Zhu, C. S. Cayman, H. Grise, P. Corcoran, M. B. Zwick, M. Franti, L. Morris, K. H. Roux, D. R. Burton, and J. M. Binley. 2006. Nature of nonfunctional envelope proteins on the surface of human immunodeficiency virus type 1. J Virol 80:2515-28.
66. Nelson, J. D., F. M. Brunel, R. Jensen, E. T. Crooks, R. M. Cardoso, M. Wang, A. Hessell, I. A. Wilson, J. M. Binley, P. E. Dawson, D. R. Burton, and M. B. Zwick. 2007. An affinity-enhanced neutralizing antibody against the membrane-proximal external region of human immunodeficiency virus type 1 gp41 recognizes an epitope between those of 2F5 and 4E10. J Virol 81:4033-43.
67. Nishimura, Y., T. Igarashi, N. Haigwood, R. Sadjadpour, R. J. Plishka, A. Buckler-White, R. Shibata, and M. A. Martin. 2002. Determination of a statistically valid neutralization titer in plasma that confers protection against simian-human immunodeficiency virus challenge following passive transfer of high-titered neutralizing antibodies. J Virol 76:2123-30.
68. Nishimura, Y., M. Shingai, R. Willey, R. Sadjadpour, W. R. Lee, C. R. Brown, J. M. Brenchley, A. Buckler-White, R. Petros, M. Eckhaus, V. Hoffman, T. Igarashi, and M. A. Martin. 2010. Generation of the pathogenic R5-tropic simian/human immunodeficiency virus SHIVAD8 by serial passaging in rhesus macaques. J Virol 84:4769-81.
69. Nkolola, J. P., H. Peng, E. C. Settembre, M. Freeman, L. E. Grandpre, C. Devoy, D. M. Lynch, A. La Porte, N. L. Simmons, R. Bradley, D.C. Montefiori, M. S. Seaman, B. Chen, and D. H. Barouch. 2010. Breadth of neutralizing antibodies elicited by stable, homogeneous clade A and clade C HIV-1 gp140 envelope trimers in guinea pigs. J Virol 84:3270-9.
70. Nyambi, P. N., M. K. Gorny, L. Bastiani, G. van der Groen, C. Williams, and S. Zolla-Pazner. 1998. Mapping of epitopes exposed on intact human immunodeficiency virus type 1 (HIV-1) virions: a new strategy for studying the immunologic relatedness of HIV-1. J Virol 72:9384-91.
71. Owens, R. J., and R. W. Compans. 1990. The human immunodeficiency virus type 1 envelope glycoprotein precursor acquires aberrant intermolecular disulfide bonds that may prevent normal proteolytic processing. Virology 179:827-33.
72. Pal, R., S. Wang, V. S. Kalyanaraman, B. C. Nair, S. Whitney, T. Keen, L. Hocker, L. Hudacik, N. Rose, A. Cristillo, I. Mboudjeka, S. Shen, T. H. Wu-Chou, D. Montefiori, J. Mascola, S. Lu, and P. Markham. 2005. Polyvalent DNA prime and envelope protein boost HIV-1 vaccine elicits humoral and cellular responses and controls plasma viremia in rhesus macaques following rectal challenge with an R5 SHIV isolate. J Med Primatol 34:226-36.
73. Pancera, M., S. Majeed, Y. E. Ban, L. Chen, C. C. Huang, L. Kong, Y. D. Kwon, J. Stuckey, T. Zhou, J. E. Robinson, W. R. Schief, J. Sodroski, R. Wyatt, and P. D. Kwong. 2010. Structure of HIV-1 gp120 with 41-interactive region reveals layered envelope architecture and basis of conformational mobility. Proc Natl Acad Sci USA 107:1166-71.
74. Pantophlet, R., and D. R. Burton. 2006. GP120: target for neutralizing HIV-1 antibodies. Annu Rev Immunol 24:739-69.
75. Phogat, S., R. T. Wyatt, and G. B. Karlsson Hedestam. 2007. Inhibition of HIV-1 entry by antibodies: potential viral and cellular targets. J Intern Med 262:26-43.
76. Poignard, P., M. Moulard, E. Golez, V. Vivona, M. Franti, S. Venturini, M. Wang, P. W. Parren, and D. R. Burton. 2003. Heterogeneity of envelope molecules expressed on primary human immunodeficiency virus type 1 particles as probed by the binding of neutralizing and normeutralizing antibodies. J Virol 77:353-65.
77. Poon, B., J. T. Safrit, H. McClure, C. Kitchen, J. F. Hsu, V. Gudeman, C. Petropoulos, T. Wrin, I. S. Chen, and K. Grovit-Ferbas. 2005. Induction of humoral immune responses following vaccination with envelope-containing, formaldehyde-treated, thermally inactivated human immunodeficiency virus type 1. J Virol 79:4927-35.
78. Racek, T., G. Jarmy, and C. Jassoy. 2006. Induction of humoral and cellular immune responses in mice by HIV-derived infectious pseudovirions. AIDS Res Hum Retroviruses 22:1162-6.
79. Rademeyer, C., P. L. Moore, N. Taylor, D. P. Martin, I. A. Choge, E. S. Gray, H. W. Sheppard, C. Gray, L. Morris, and C. Williamson. 2007. Genetic characteristics of HIV-1 subtype C envelopes inducing cross-neutralizing antibodies. Virology 368:172-181.
80. Rerks-Ngarm, S., P. Pitisuttithum, S. Nitayaphan, J. Kaewkungwal, J. Chiu, R. Paris, N. Premsri, C. Namwat, M. de Souza, E. Adams, M. Benenson, S. Gurunathan, J. Tartaglia, J. G. McNeil, D. P. Francis, D. Stablein, D. L. Birx, S. Chunsuttiwat, C. Khamboonruang, P. Thongcharoen, M. L. Robb, N. L. Michael, P. Kunasol, and J. H. Kim. 2009. Vaccination with ALVAC and AIDSVAX to prevent HIV-1 infection in Thailand. N Engl J Med 361:2209-20.
81. Sanders, R. W., M. Venturi, L. Schiffner, R. Kalyanaraman, H. Katinger, K. O. Lloyd, P. D. Kwong, and J. P. Moore. 2002. The mannose-dependent epitope for neutralizing antibody 2G12 on human immunodeficiency virus type 1 glycoprotein gp120. J Virol 76:7293-305.
82. Sanders, R. W., M. Vesanen, N. Schuelke, A. Master, L. Schiffner, R. Kalyanaraman, M. Paluch, B. Berkhout, P. J. Maddon, W. C. Olson, M. Lu, and J. P. Moore. 2002. Stabilization of the soluble, cleaved, trimeric form of the envelope glycoprotein complex of human immunodeficiency virus type 1. J Virol 76:8875-89.
83. Sather, D. N., J. Armann, L. K. Ching, A. Mavrantoni, G. Sellhorn, Z. Caldwell, X. Yu, B. Wood, S. Self, S. Kalams, and L. Stamatatos. 2008. Factors Associated with the Development of Cross-Reactive Neutralizing Antibodies during HIV-1 Infection. J. Virol.
84. Sattentau, Q. J. 1995. Conservation of HIV-1 gp120 neutralizing epitopes after formalin inactivation. Aids 9:1383-5.
85. Sattentau, Q. J., and J. P. Moore. 1995. Human immunodeficiency virus type 1 neutralization is determined by epitope exposure on the gp120 oligomer. J Exp Med 182:185-96.
86. Scanlan, C. N., G. E. Ritchie, K. Baruah, M. Crispin, D. J. Harvey, B. B. Singer, L. Lucka, M. R. Wormald, P. Wentworth, Jr., N. Zitzmann, P. M. Rudd, D. R. Burton, and R. A. Dwek. 2007. Inhibition of Mammalian Glycan Biosynthesis Produces Non-self Antigens for a Broadly Neutralising, HIV-1 Specific Antibody. J Mol Biol 372:16-22.
87. Scheid, J. F., H. Mouquet, N. Feldhahn, M. S. Seaman, K. Velinzon, J. Pietzsch, R. G. Ott, R. M. Anthony, H. Zebroski, A. Hurley, A. Phogat, B. Chakrabarti, Y. Li, M. Connors, F. Pereyra, B. D. Walker, H. Wardemann, D. Ho, R. T. Wyatt, J. R. Mascola, J. V. Ravetch, and M. C. Nussenzweig. 2009. Broad diversity of neutralizing antibodies isolated from memory B cells in HIV-infected individuals. Nature 458:636-40.
88. Schulke, N., M. S. Vesanen, R. W. Sanders, P. Zhu, M. Lu, D. J. Anselma, A. R. Villa, P. W. Parren, J. M. Binley, K. H. Roux, P. J. Maddon, J. P. Moore, and W. C. Olson. 2002. Oligomeric and conformational properties of a proteolytically mature, disulfide-stabilized human immunodeficiency virus type 1 gp140 envelope glycoprotein. J Virol 76:7760-76.
89. Skehel, J. J., P. M. Bayley, E. B. Brown, S. R. Martin, M. D. Waterfield, J. M. White, I. A. Wilson, and D.C. Wiley. 1982. Changes in the conformation of influenza virus hemagglutinin at the pH optimum of virus-mediated membrane fusion. Proc Natl Acad Sci USA 79:968-72.
90. Skountzou, I., F. S. Quan, S. Gangadhara, L. Ye, A. Vzorov, P. Selvaraj, J. Jacob, R. W. Compans, and S. M. Kang. 2007. Incorporation of glycosylphosphatidylinositol-anchored granulocyte-macrophage colony-stimulating factor or CD40 ligand enhances immunogenicity of chimeric simian immunodeficiency virus-like particles. J Virol 81:1083-94.
91. Srivastava, I. K., E. Kan, Y. Sun, V. A. Sharma, J. Cisto, B. Burke, Y. Lian, S. Hilt, Z. Biron, K. Hartog, L. Stamatatos, R. H. Cheng, J. B. Ulmer, and S. W. Barnett. 2007. Comparative evaluation of trimeric envelope glycoproteins derived from subtype C and B HIV-1 R5 isolates. Virology:Epub ahead of print.
92. Stephens, D. M., J. W. Eichberg, N. L. Haigwood, K. S. Steimer, D. Davis, and P. J. Lachmann. 1992. Antibodies are produced to the variable regions of the external envelope glycoprotein of human immunodeficiency virus type 1 in chimpanzees infected with the virus and baboons immunized with a candidate recombinant vaccine. J Gen Virol 73 (Pt 5):1099-106.
93. Stoute, J. A., M. Slaoui, D. G. Heppner, P. Momin, K. E. Kester, P. Desmons, B. T. Wellde, N. Garcon, U. Krzych, and M. Marchand. 1997. A preliminary evaluation of a recombinant circumsporozoite protein vaccine against Plasmodium falciparum malaria. RTS,S Malaria Vaccine Evaluation Group. N Engl J Med 336:86-91.
94. Tomaras, G. D., N. L. Yates, P. Liu, L. Qin, G. G. Fouda, L. L. Chavez, A. C. Decamp, R. J. Parks, V. C. Ashley, J. T. Lucas, M. Cohen, J. Eron, C. B. Hicks, H. X. Liao, S. G. Self, G. Landucci, D. N. Forthal, K. J. Weinhold, B. F. Keele, B. H. Hahn, M. L. Greenberg, L. Morris, S. S. Karim, W. A. Blattner, D.C. Montefiori, G. M. Shaw, A. S. Perelson, and B. F. Haynes. 2008. Initial B-cell responses to transmitted human immunodeficiency virus type 1: virion-binding immunoglobulin M (IgM) and IgG antibodies followed by plasma anti-gp41 antibodies with ineffective control of initial viremia. J Virol 82:12449-63.
95. Vaine, M., S. Lu, and S. Wang. 2009. Progress on the induction of neutralizing antibodies against HIV type 1 (HIV-1). BioDrugs 23:137-53.
96. Vaine, M., S. Wang, E. T. Crooks, P. Jiang, D.C. Montefiori, J. Binley, and S. Lu. 2008. Improved induction of antibodies against key neutralizing epitopes by HIV-1 gp120 DNA prime-protein boost vaccination compared to gp120 protein only vaccination. J Virol 82:7369-78.
97. Vaine, M., S. Wang, A. Hackett, J. Arthos, and S. Lu. 2010. Antibody responses elicited through homologous or heterologous prime-boost DNA and protein vaccinations differ in functional activity and avidity. Vaccine 28:2999-3007.
98. VanCott, T. C., J. R. Mascola, R. W. Kaminski, V. Kalyanaraman, P. L. Hallberg, P. R. Burnett, J. T. Ulrich, D. J. Rechtman, and D. L. Birx. 1997. Antibodies with specificity to native gp120 and neutralization activity against primary human immunodeficiency virus type 1 isolates elicited by immunization with oligomeric gp160. J Virol 71:4319-30.
99. Verrier, F., C. Moog, F. Barre-Sinoussi, E. Van der Ryst, C. Spenlehauer, and M. Girard. 2000. Macaque immunization with virions purified from a primary isolate of the human immunodeficiency virus type 1 induced enhancement antibodies. Bull Acad Natl Med 184:67-84.
100. Vzorov, A. N., D. Lea-Fox, and R. W. Compans. 1999. Immunogenicity of full length and truncated SIV envelope proteins. Viral Immunol 12:205-15.
101. Walker, L. M., S. K. Phogat, P. Y. Chan-Hui, D. Wagner, P. Phung, J. L. Goss, T. Wrin, M. D. Simek, S. Fling, J. L. Mitcham, J. K. Lehrman, F. H. Priddy, O. A. Olsen, S. M. Frey, P. W. Hammond, S. Kaminsky, T. Zamb, M. Moyle, W. C. Koff, P. Poignard, and D. R. Burton. 2009. Broad and potent neutralizing antibodies from an African donor reveal a new HIV-1 vaccine target. Science 326:285-9.
102. Wang, S., R. Pal, J. R. Mascola, T. H. Chou, I. Mboudjeka, S. Shen, Q. Liu, S. Whitney, T. Keen, B. C. Nair, V. S. Kalyanaraman, P. Markham, and S. Lu. 2006. Polyvalent HIV-1 Env vaccine formulations delivered by the DNA priming plus protein boosting approach are effective in generating neutralizing antibodies against primary human immunodeficiency virus type 1 isolates from subtypes A, B, C, D and E. Virology 350:34-47.
103. Willey, R., M. C. Nason, Y. Nishimura, D. A. Follmann, and M. A. Martin. 2010. Neutralizing antibody titers conferring protection to macaques from a simian/human immunodeficiency virus challenge using the TZM-bl assay. AIDS Res Hum Retroviruses 26:89-98.
104. Yuan, W., J. Bazick, and J. Sodroski. 2006. Characterization of the multiple conformational States of free monomeric and trimeric human immunodeficiency virus envelope glycoproteins after fixation by cross-linker. J Virol 80:6725-37.
105. Yuan, W., S. Craig, X. Yang, and J. Sodroski. 2005. Inter-subunit disulfide bonds in soluble HIV-1 envelope glycoprotein trimers. Virology 332:369-83.
106. Yuan, W., X. Li, M. Kasterka, M. K. Gorny, S. Zolla-Pazner, and J. Sodroski. 2009. Oligomer-specific conformations of the human immunodeficiency virus (HIV-1) gp41 envelope glycoprotein ectodomain recognized by human monoclonal antibodies. AIDS Res Hum Retroviruses 25:319-28.

1-187. (canceled)

188. An immunogenic composition comprising: a virus-like particle having a surface, said surface comprising immunodeficiency virus trimeric gp120/gp41 Env protein bound thereto, wherein said composition is substantially free from uncleaved gp160 protein, and wherein said composition is capable of inducing production of neutralizing antibodies against said immunodeficiency virus in a subject administered said composition.

189. The composition of claim 188, wherein the trimeric gp120/gp41 Env protein is cleaved at amino acid residue 511 in the gp160 precursor.

190. The composition of claim 188, wherein said surface comprises substantially only immunodeficiency virus trimeric gp120/gp41 Env protein bound thereto.

191. The composition of claim 188, wherein the gp160 is not cleaved at either amino acid residues 504 or 511.

192. The composition of claim 188, wherein the virus-like particle further substantially lacks gp41 stumps bound thereto, wherein the gp41 is unlinked to gp120.

193. The composition of claim 188, wherein said immunodeficiency virus is HIV-1.

194. The composition of claim 188, wherein said HIV-1 is selected from the group consisting of clade A, B, C, D, CRF01_AE, CRF02_AG, F1, F2, G, H, J, K N, O, P, U and inter-clade recombinant versions thereof.

195. The composition of claim 188, wherein said HIV-1 is selected from the group consisting of clades B, C, and CRF01_AE.

196. A method of making an immunogenic composition comprising: obtaining a plurality of immunodeficiency virus-like particles having a surface comprising trimeric gp120/gp41 Env protein and uncleaved gp160 protein thereon; and contacting said virus-like particles with an enzyme that substantially and selectively removes the uncleaved gp160 from the surface to generate purified virus-like particles having a surface substantially free from uncleaved gp160 protein bound thereto.

197. The method of claim 196, wherein said contacting generates purified virus-like particles comprising substantially only trimeric gp120/gp41 Env protein.

198. The method of claim 196, wherein the enzyme is a protease.

199. The method of claim 196, wherein the protease is any of chymotrypsin, trypsin, pepsin, elastase, papain, subtilisin, cathepsin C, pyroglutamate aminopeptidase, plasmin, and bromelain.

200. The method of claim 196, wherein the protease includes chymotrypsin.

201. The method of claim 196, wherein the trimeric gp120/gp41 Env protein is proteolytically cleaved at amino acid residue 511 of a gp160 precursor.

202. The method of claim 196, wherein the gp160 is proteolytically uncleaved at amino acid residues 504 or 511.

203. The method of claim 196, wherein the purified virus-like particles further substantially lack gp41 stumps bound thereto, wherein the gp41 is unlinked to gp120.

204. The method of claim 196, wherein said immunodeficiency virus is HIV-1.

205. A method of immunizing a mammal against an immunodeficiency virus comprising administering an effective amount of an immunogenic composition comprising: a virus-like particle having a surface, said surface comprising immunodeficiency virus trimeric gp120/gp41 Env protein bound thereto, wherein said composition is substantially free from uncleaved gp160 protein, and wherein said composition is capable of inducing production of neutralizing antibodies against said immunodeficiency virus in a subject administered said composition.

206. The method of claim 205, further comprising adminstering an adjuvant.

207. The method of claim 206, wherein the adjuvant administered is selected from the group consisting of: Ribi, QS21, Carbopol, CpG, Ribi, AS01, AS02, AS03, AS04, Quil A, MF-59, Freund's, incomplete Freund's, MPL, muramyl dipeptides, detoxified lipid A, PCPP, SAF-1, polymethylmethacrylate nanoparticles (PMMA), IL-12, cholera toxin B, ISCOMS, saponins, TDM, CWS emulsion, poly I:C, virosomes, alum, alhydrogel, CD40L, BAFF, APRIL, and C3d.

208. The method of claim 205, further comprising administering a second composition of trimeric gp120/gp41 proteins, wherein the trimeric gp120/gp41 Env proteins administered have different amino acid sequences.

209. A method of selecting neutralizing antibodies against Env protein comprising sorting memory B cells from an immunodeficiency virus-infected subject, contacting antibodies produced by the sorted B cells with soluble trimeric gp120/gp41 or a particle having substantially only trimeric gp120/gp41 Env protein bound thereto, and identifying the B cells that produce neutralizing antibodies against said trimeric gp120/gp41
